(19) United States
(12) Patent Application Publication
(10) Pub. No.: US 2005/0215473 A1
(43) Pub. Date: Sep. 29, 2005

(54) Prophylactic and therapeutic uses of FGF-20 in radiation protection

(76) Inventors: Enrique Alvarez, Clinton, CT (US); Timothy MacLachlan, Cheshire, CT (US); Bisni Narayanan, Oaklyn, NJ (US); William Hahne, Madison, CT (US)

Correspondence Address: Jenell Lawson;Intellectual Property, CuraGen Corporation, 555 Long Wharf Drive, New Haven, CT, 06551, US

(21) Appl. No.: 10/980,695

(22) Filed: Nov. 3, 2004

Related U.S. Application Data
(63) Continuation-in-part of application No. 10/842,179, filed on May 10, 2004.
(60) Provisional application No. 60/469,353, filed on May 9, 2003.
(60) Provisional application No. 60/565,362, filed on Apr. 26, 2004.
(60) Provisional application No. 60/544,308.

(51) Int. Cl. A61K 38/17

(52) U.S. Cl. 514/12

(57) ABSTRACT

The present invention relates to methods of stimulating stem cell proliferation and engraftment, and methods of preventing and/or treating disorders associated with radiation exposure, chemotherapy, exposed to chemical/biological warfare agents, and/or any other insults affecting rapidly proliferating tissues in a body, or one or more symptoms thereof. In particular, the present invention provides methods of stimulating stem cell proliferation and/or engraftment and methods of preventing and/or treating disorders associated with one or more insults affecting rapidly proliferating tissues in a body (e.g., radiation exposure, chemical and/or biological insults) or one or more symptoms thereof by administering to a subject a composition comprising a Fibroblast Growth Factor-20 (FGF-20) protein, or its fragments, derivatives, variants, homologs, analogs, or a combination thereof.

This application is a continuation-in-part of the U.S. patent application Ser. No. 10/842,179, filed May 10, 2004. This application also claims the benefit of U.S. Provisional Application No. ______, Attorney Docket No. Cura-57 RP2, filed Sep. 22, 2004, entitled “PROPHYLACTIC AND THERAPEUTIC USES OF FGF-20 IN RADIATION PROTECTION.” The content of each is incorporated herein by reference in its entirety.

### 1. FIELD OF INVENTION

The present invention relates to methods of stimulating stem cell proliferation and engraftment, and methods of preventing and/or treating disorders associated with radiation exposure, chemotherapy, exposure to chemical/biological warfare agents, and/or any other insults affecting rapidly proliferating tissues in a body, or one or more symptoms thereof. In particular, the present invention provides methods of stimulating stem cell proliferation and/or engraftment and methods of preventing and/or treating disorders associated with one or more insults affecting rapidly proliferating tissues in a body (e.g., radiation exposure, chemical and/or biological insults) or one or more symptoms thereof by administering to a subject a composition comprising a Fibroblast Growth Factor-20 (FGF-20) protein, or its fragments, derivatives, variants, homologs, analogs, or a combination thereof.

### 2. BACKGROUND OF THE INVENTION

The FGF family consists of more than 20 members, each containing a conserved amino acid core (see, e.g., Powers et al., Endocr. Relat. Cancer, 7(3):65-197 (2000)). FGFs regulate diverse cellular functions such as growth, survival, apoptosis, motility, and differentiation (see, e.g., Szebenyi et al., Int. Rev. Cytol., 185:45-106 (1999)). Members of the FGF family are involved in various physiological and pathological processes during embryogenesis and adult life, including morphogenesis, limb development, tissue repair, inflammation, angiogenesis, and tumor growth and invasion (see, e.g., Powers et al., Endocr. Relat. Cancer, 7(3):165-197 (2000); or Szebenyi et al., Int. Rev. Cytol. 185:45-106 (1999)). Through a homology-based genomic mining process, a new member of the FGF family, FGF-20, has been identified (see U.S. patent application Ser. No. 09/494,585, filed Jan. 13, 2000, and Ser. No. 09/609,543, filed Jul. 3, 2000, the disclosures of which are incorporated herein by reference in their entireties).

A single exposure to ionizing radiation can produce immediate effects on tissue through free radical generation and often results in radiation sickness. Radiation-induced lethality depends on dose exposure. Three main syndromes are associated with single-dose exposure to radiation: cardiovascular/central nerve system (CNS) syndrome, gastrointestinal (GI) syndrome, and hematopoietic (bone marrow) syndrome (see, e.g., Coleman et al., Radiat. Res., 159(6):812-834 (2003)). The time of death, depending on the dose of exposure, is within hours for cardiovascular/central nerve system (CNS) syndrome, within 3-10 days for GI syndrome, and 30-60 days for hematopoietic syndrome. Other symptoms associated with radiation sickness include, but are not limited to, nausea, vomiting, diarrhea, skin burns and sores, fatigue, dehydration, inflammation, hair loss, neutropenia, ulceration of the oral mucosa and GI system, xerostomia, and bleeding (e.g., bleeding from the nose, mouth and rectum). A common underlying cause for the symptoms associated with radiation sickness is the direct effect of ionizing radiation on stem cell precursor cells. Mitotically active hematopoietic progenitors are unable to divide after a whole body exposure of 2-3 Gy, resulting in lymphopenia, thrombocytopenia, anemia, bone marrow atrophy, followed by infection, bleeding and poor wound healing, which contribute to lethality of the hematopoietic syndrome. Radiation at doses of less than 2 Gy induces mild cytopenias without significant bone marrow damage (see e.g., Geiselhart et al., J. Immunol. 166(5):3019-3027 (2001)). Peripheral blood lymphopenia may develop within the first 6-24 hrs after a moderate to high dose exposure (see e.g., Mackall et al., Blood 97(5):1491-1497 (2001)). Neutropenia (a decrease in the number of neutrophils resulting in increased risk of infection) and gastrointestinal mucositis represent two of the most significant causes of mortality resulting from ionizing radiation. Vesicant agents such as mustard gas, like radiation, cause similar effects at the cellular level; their use in combination will have a geometric effect on morbidity.

Palliative medical interventions, such as blood cell replacements, antibiotics, cytokines, and in high-dose cases, hematopoietic stem cell transplants, could increase overall survival. Biological modification of radiation response has been an active area of investigation for many years. The development of both active radiation sensitizers and protectants could also provide therapeutic advantages to patients undergoing radiation therapy (see, e.g., Cancer Chemotherapeutic Agents, 1995:501-527). Additional indications for radioprotectants include, e.g., the protection of personnel involved in accidental exposure to ionizing radiation in, e.g., an industrial, medical, or military setting, and protection of personnel exposed to radiation in the event of nuclear accident or terrorist attack.

Treatment with certain members of the FGF family have been shown to improve survival and hematopoietic recovery in mice following ionizing radiation to the whole body (see, e.g., Cytokine, 9(1):59-65 (1997); Acta Oncol., 36(3):337-340 (1997); Acta Oncol., 34(3):435-438 (1995); Radiat. Res., 150(2):204-11 (1998); and Am. J. Clin. Oncol., 24(5):491-5 (2001)). Acidic FGF (aFGF or FGF-1,1-24 mg) increases the survival of C3H/HeCNR mice receiving 840 centi-Grey (cGy) of ionizing radiation to the whole body (see Cytokine, 9(1):59-65 (1997)). The radioprotective effects of three other FGF family members, basic FGF (bFGF or FGF-2), keratinocyte growth factors 1 and 2 (KGF-1 or FGF-7, and KFG-2 or FGF-10), have been examined in both C3H/He and BalbC mice (see Am. J. Clin. Oncol., 24(5):491-5 (2001)). These studies showed that the LD₅₀ for ionizing radiation was increased by approximately 100 cGy with a single dose (6 μg) of growth factor.

Ionizing radiation is named such because of the ability of the radiation particles to generate reactive ions when bombarded against another molecule within the cell or elsewhere. As one of the most common molecules within a cell is water, H₂O, very often the radicals generated are a form of this molecule's breakdown (see, e.g., Int. J. Radiat. Biol, 65:27-33 (1994)). The reactive oxygen species (ROS) include, but are not limited to, superoxide (O₂⁻), hydroxyl (OH) and hydrogen peroxide (H₂O₂) (see Proc. Natl. Acad. Sci. USA, 78:1001-1003 (1981)). While these molecular intermediates are useful to the cell, for example, the production of energy by means of the electron transport pathway in the mitochondria, an overabundance of ROS can cause tissue destruction due to their highly reactive nature. Breakage of genomic DNA, depolarization of the mitochondrial membrane and alteration of proteins are among the types of damage that such radicals can cause. The body's first line of defense against ROS is enzymes that scavenge the radicals to less reactive species. These include the superoxide dismutases (MnSOD, CuZnSOD, extracellular-SOD), glutathione peroxidase and genes induced by the transcription factor Nrf2 (see Free Radic. Biol. Med., 17:389-395 (1994); FASEB J., 7:361-368 (1993); Proc. Natl. Acad. Sci. USA 94:5361-5366). Indeed, many of these proteins have been shown to be radioprotective to cells when overexpressed (see Free Radic. Biol. Med., 17:389-395 (1994), and FASEB J., 7:361-368 (1993)). In addition, some other members of the fibroblast growth factor family, such as KGF, have been shown to affect the expression of Nrf2 (see Mol. Cell Biol., 22:5492-5505 (2002)).

There are still great clinical needs for better radiation protection agents that can provide effective protection from or treatment of disorders associated with radiation exposure and other insults affecting rapidly proliferating tissues, or one or more symptoms thereof. The development of active radiation protectants could also provide therapeutic advantages to patients undergoing radiation therapy, and protection of first responders or military personnel exposed to ionizing radiation in an industrial or military setting, or exposure of the civilian population to ionizing radiation resulting from nuclear accidents or terrorism.

Citation or discussion of a reference herein shall not be construed as an admission that such is prior art to the present invention.

### 3. SUMMARY OF THE INVENTION

The present invention provides methods of preventing or treating one or more disorders associated with radiation exposure(s), chemotherapy, exposure(s) to chemical and/or biological warfare agents with radiomimetic properties, and/or exposure(s) to any other insults affecting rapidly proliferating tissues in a body, or one or more symptoms thereof by administering to a subject in need thereof a composition comprising one or more CG53135 proteins. In one embodiment, the disorder to be prevented and/or treated is a disorder of hematopoiesis, including but is not limited to, anemia, leukopenia (e.g., netropenia), thrombocytopenia, pancytopenia, and a clotting disorder. In another embodiment, the disorder to be prevented and/or treated is bone marrow failure. In another embodiment, the disorder to be prevented and/or treated is graft-versus-host disease. In another embodiment, the disorder to be prevented or treated is alimentary mucositis, including but is not limited to, oral mucositis, esophagitis, stomatitis, enteritis, and proctitis. In another embodiment, the disorder to be prevented and/or treated is radiation induced prostatitis, virginitis, and/or urethritis. In another embodiment, the disorder to be prevented or treated is a cardiovascular and/or central nervous system syndrome. In some embodiments, more than one disorders described above may be prevented and/or treated by the methods of the invention. In some embodiments, the symptoms associated with an insult affecting rapidly proliferating tissues (e.g., radiation, chemotherapy, and chemical/biological warfare agents) include, but are not limited to, diarrhea, skin burn, sores, fatigue, dehydration, inflammation, hair loss, ulceration of oral mucosa, xerostomia, and bleeding (e.g., from the nose, mouth or rectum).

The present invention is based, in part, upon the inventors' discoveries that CG53135 protects rapidly proliferating tissues, such as hematopoietic and gastrointestinal tissues, from insults such as radiation exposure(s), chemotherapy, and exposure(s) to one or more chemical and/or biological warfare agents, and CG53135 stimulates proliferation and engraftment of stem cells such as hematopoietic stem cells and/or gastrointestinal stem cells. While not limited by any theory, CG53135 is believed to protect stem cells associated with the regenerative capacities of the proliferating tissues from the adverse effects of cytotoxic agents. This general protection ultimately leads to the amelioration of symptoms and/or improvement of morbidity and mortality associated with insults affecting rapidly proliferating tissues.

Accordingly, the present invention also provides methods of upregulating oxygen scavenging pathways in a subject, where the methods comprises administering to the subject a composition comprising one or more CG53135 proteins. In one embodiment, the oxygen scavenging pathways comprise one or more superoxide dismutases (“SOD”). In another embodiment, the oxygen scavenging pathways comprise genes selected from the group consisting of an extracelluar signal regulated kinase (“ERK”), an adhesion related kinase (“AKT”), a superoxide dismutase, cyclooxygenase-2 (“COX-2”), and NF-E2-related factor 2 (“Nrf-2”).

The present invention further provides methods of stimulating secretion of one or more endogenous cytokines or endogenous chemokines from cells of a subject, where the method comprises administering to the subject a composition comprising one or more CG53135 proteins.

The present invention provides methods of stimulating proliferation of hematopoietic stem cells and/or gastrointestinal stem cells of a subject, where the method comprises administering to the subject a composition comprising one or more CG53135 proteins.

The present invention also provides methods of optimizing engraftment of hematopoietic stem cells in a subject, where the method comprises administering to the subject a composition comprising one or more CG53135 proteins.

In one embodiment, the present invention provides a method of protecting and/or regenerating hematopoietic tissues of a subject exposed to an insult affecting rapidly proliferating tissues (such as radiation, chemotherapy, and chemical/biological warfare agents with radiomimetic properties) comprising administering to the subject a therapeutically effective amount of a composition comprising one or more CG53135 proteins. In another embodiment, the present invention provides a method of treating leukopenia (e.g., neutropenia) of a subject exposed to an insult affecting rapidly proliferating tissues (such as radiation, chemotherapy, and chemical/biological warfare agents with radiomimetic properties) comprising administering to the subject a therapeutically effective amount of a composition comprising one or more CG53135 proteins.

In another embodiment, the present invention provides a method of protecting and/or regenerating gastrointestinal tissues of a subject exposed to an insult affecting rapidly proliferating tissues (such as radiation, chemotherapy, and chemical/biological warfare agents with radiomimetic properties) comprising administering to the subject a therapeutically effective amount of a composition comprising one or more CG53135 proteins. In one embodiment, the present invention provides a method of treating gastrointestinal mucositis of a subject exposed to an insult affecting rapidly proliferating tissues (such as radiation, chemotherapy, and chemical/biological warfare agents with radiomimetic properties) comprising administering to the subject a therapeutically effective amount of a composition comprising one or more CG53135 proteins.

In some embodiments, the present invention provides a method of preventing and/or treating a disorder (e.g., alimentary mucositis, bone marrow failure, radiation induced prostatitis, virginitis and/or urethritis, a disorder of hematopoiesis, or a cardiovascular/central nervous system syndrome) or ameliorating a symptom (e.g., diarrhea, skin burn, sores, fatigue, dehydration, inflammation, hair loss, ulceration of oral mucosa, xerostomia, and bleeding) associated with an insult affecting rapidly proliferating tissues (such as radiation, chemotherapy, and chemical/biological warfare agents with radiomimetic properties) comprising administering to a subject in need thereof a prophylactically or therapeutically effective amount of a composition comprising one or more CG53135 proteins. In one embodiment, a composition comprising one or more CG53135 proteins is administered to a subject prior to the subject's exposure to the insult. In another embodiment, a composition comprising one or more CG53135 proteins is administered to a subject after the subject's exposure to the insult, but prior to any disorder associated with the insult, or a symptom thereof developed in the subject. In another embodiment, a composition comprising one or more CG53135 proteins is administered to a subject after one or more disorders associated with the insult or symptoms thereof developed in the subject. In another embodiment, a composition comprising one or more CG53135 proteins is administered a subject in need thereof both prior to the development of any radiation associated disorder and/or symptom (e.g., prior to the occurrence of the insult, and/or after the occurrence of the insult but prior to the development of any disorder and/or symptom) and after the development of a radiation associated disorder and/or symptom. In yet another embodiment, a composition comprising one or more CG53135 proteins is administered to a subject who is at risk of exposing to an insult affecting rapidly proliferating tissues (such as radiation, chemotherapy, and chemical/biological warfare agents with radiomimetic properties). In a specific embodiment, a composition comprising one or more CG53135 proteins is administered to a subject in need thereof no more than 24 hours, 20 hours, 15 hours, 10 hours, or 5 hours prior to the subject's exposure to an insult affecting rapidly proliferating tissues (e.g., radiation, chemotherapy, and chemical/biological warfare agents with radiomimetic properties). In another embodiment, a composition comprising one or more CG53135 proteins is administered to a subject in need thereof 3 days, 2 days, 1 day prior to exposure to radiation (day −3, −2, and −1), the day exposed to radiation (day 0), and the day after exposure to the radiation (day 1), respectively. In yet another embodiment, a composition comprising one or more CG53135 proteins is administered to a subject in need thereof on day −1, 0, and 1, respectively. Many more dosing schedules can be used, and such schedules are encompassed by the present invention.

In another embodiment, the present invention provides a method of improving survival of subjects exposed to an insult affecting rapidly proliferating tissues (such as radiation, chemotherapy, and chemical/biological warfare agents with radiomimetic properties) comprising administering to the subjects a prophylactically or therapeutically effective amount of a composition comprising one or more CG53135 proteins. The therapeutically effective dose may be a single dose, two doses or more than two doses of a composition comprising one or more CG53135 proteins.

In another embodiment, a single prophylactic dose of a composition comprising one or more CG53135 proteins is administered to a subject followed by an insult affecting rapidly proliferating tissues (such as radiation, chemotherapy, and chemical/biological warfare agents with radiomimetic properties), where such prophylactic dose causes a defined, short acting proliferative effect on various compartments in the proliferating tissues (e.g., intestinal villi). In another embodiment, more than a single prophylactic dose, which may be two or more than two doses of a composition comprising one or more CG53135 proteins, is administered to a subject exposed to an insult affecting rapidly proliferating tissues (such as radiation, chemotherapy, and chemical/biological warfare agents with radiomimetic properties) to preventing, treating or ameliorating a symptom associated with the insult.

In some embodiments, an insult affecting rapidly proliferating tissues is radiation exposure. In some embodiments, an insult affecting rapidly proliferating tissues is one or more alkylating agents, one or more vesicant agents (e.g., mustard agents), or one or more other chemotherapeutic agents, or a combination thereof. In some embodiments, an insult affecting rapidly proliferating tissues is a radiation exposure in combination with one or more alkylating agents, one or more mustard agents, or one or more other chemotherapeutic agents.

In some embodiments, a composition comprising one or more CG53135 proteins is used in combination with one or more other therapies known in the art to prevent, treat, or ameliorate one or more symptoms associated with an insult affecting rapidly proliferating tissues (such as radiation, chemotherapy, and chemical/biological warfare agents with radiomimetic properties).

Pharmaceutical compositions, formulations, and kits are also encompassed by the present invention.

3.1. Terminology

As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs. In a preferred embodiment, a CG53135 protein retains at least some biological activity of FGF-20. As used herein, the term “biological activity” means that a CG53135 protein possesses some but not necessarily all the same properties of (and not necessarily to the same degree as) FGF-20.

A member (e.g., a protein and/or a nucleic acid encoding the protein) of the CG53135 family may further be given an identification name. For example, CG53135-01 (SEQ ID NOs:1 and 2) represents the first identified FGF-20 (see U.S. patent application Ser. No. 09/494,585); CG53135-05 (SEQ ID NOs:8 and 2) represents a codon-optimized, full length FGF-20 (i.e., the nucleic acid sequence encoding FGF-20 has been codon optimized, but the amino acid sequence has not been changed from the originally identified FGF-20); CG53135-12 (SEQ ID NOs:21 and 22) represent a single nucleotide polymorphism (“SNP”) of FGF-20 where one amino acid in CG53135-12 is different from SEQ ID NO:2 (the aspartic acid at position 206 is changed to asparagine, “²⁰⁶D? N”). Some members of the CG53135 family may differ in their nucleic acid sequences but encode the same CG53135 protein, e.g., CG53135-01, CG53135-03, and CG53135-05 all encode the same CG53135 protein. An identification name may also be an in-frame clone (“IFC”) number, for example, IFC 250059629 (SEQ ID NOs:33 and 34) represents amino acids 63-196 of the full length FGF-20 (cloned in frame in a vector). Table 1A shows a summary of some of the CG53135 family members. In one embodiment, the invention includes a variant of FGF-20 protein, in which some amino acids residues, e.g., no more than 1%, 2%, 3%, 5%, 10% or 15% of the amino acid sequence of FGF-20 (SEQ ID NO:2), are changed. In another embodiment, the invention includes nucleic acid molecules that can hybridize to FGF-20 under stringent hybridization conditions.

TABLE 1A
Summary of some of the CG53135 family members

| | SEQ ID NO | |
|---|---|---|
| Name | (DNA/Protein) | Brief Description |
| CG53135-01 | 1 and 2 | FGF-20 wild type, stop codon removed |
| CG53135-02 | 3 and 4 | Codon optimized, amino acids 2-54 (as numbered in SEQ ID |
| | | NO: 2) were removed |
| CG53135-03 | 5 and 2 | FGF-20 wild type |
| CG53135-04 | 6 and 7 | Amino acids 20-51 (as numbered in SEQ ID NO: 2) were removed, |
| | | also valine at position 85 is changed to alanine (“⁸⁵V? A”) |
| CG53135-05 | 8 and 2 | Codon optimized, full length FGF-20 |
| CG53135-06 | 9 and 10 | Amino acids 20-51 (as numbered in SEQ ID NO: 2) were removed |
| CG53135-07 | 11 and 12 | Protein consisting of amino acids 1-18 (as numbered in SEQ ID |
| | | NO: 2) |
| CG53135-08 | 13 and 14 | Protein consisting of amino acids 32-52 (as numbered in SEQ ID |
| | | NO: 2) |
| CG53135-09 | 15 and 16 | Protein consisting of amino acids 173-183 (as numbered in SEQ |
| | | ID NO: 2) |
| CG53135-10 | 17 and 18 | Protein consisting of amino acids 192-211 (as numbered in SEQ |
| | | ID NO: 2) |
| CG53135-11 | 19 and 20 | Protein consisting of amino acids 121-1 37 (as numbered in SEQ |
| | | ID NO: 2) |
| CG53135-12 | 21 and 22 | FGF-20 SNP, aspartic acid at position 206 is changed to |
| | | asparagines (“²⁰⁶D? N”) as compared to CG53135-01 |
| CG53135-13 | 23 and 24 | CG53135-05 minus first 2 amino acids at the N-terminus |
| CG53135-14 | 25 and 26 | CG53135-05 minus first 8 amino acids at the N-terminus |
| CG53135-15 | 27 and 28 | CG53135-05 minus first 11 amino acids at the N-terminus |
| CG53135-16 | 29 and 30 | CG53135-05 minus first 14 amino acids at the N-terminus |
| CG53135-17 | 31 and 32 | CG53135-05 minus first 23 amino acids at the N-terminus |
| IFC 250059629 | 33 and 34 | In frame clone, open reading frame comprising a nucleotide |
| | | sequence encoding amino acids 63-196 of FGF-20 (SEQ ID NO: 2) |
| IFC 250059669 | 35 and 36 | In frame clone, open reading frame comprising a nucleotide |
| | | sequence encoding amino acids 63-211 of FGF-20 (SEQ ID NO: 2) |
| IFC 317459553 | 37 and 38 | In frame clone, open reading frame comprising a nucleotide |
| | | sequence encoding amino acids 63-194 of FGF-20 (SEQ ID NO: 2) |
| | | with ¹⁵⁹G? E |
| IFC 317459571 | 39 and 40 | In frame clone, open reading frame comprising a nucleotide |
| | | sequence encoding amino acids 63-194 of FGF-20 (SEQ ID NO: 2) |
| IFC 250059596 | 41 and 10 | In frame clone, open reading frame comprising a nucleotide |
| | | sequence encoding amino acids 1-19 and 52-211 of FGF-20 (SEQ |
| | | ID NO: 2) |
| IFC 316351224 | 41 and 10 | In frame clone, open reading frame comprising a nucleotide |
| | | sequence encoding amino acids 1-19 and 52-211 of FGF-20 (SEQ |
| | | ID NO: 2). |

As used herein, the term “effective amount” refers to the amount of a therapy (e.g., a composition comprising one or more CG53135 proteins) which is sufficient to reduce and/or ameliorate the severity and/or duration of a disease or disorder associated with an insult affecting a rapidly proliferating tissue (such as radiation, chemotherapy, and chemical/biological warfare agents) or one or more symptoms thereof, prevent the advancement of said disease or disorder, cause regression of said disease or disorder, prevent the recurrence, development, or onset of one or more symptoms associated with the insult, or enhance or improve the prophylactic or therapeutic effect(s) of another therapy (e.g., prophylactic or therapeutic agent).

As used herein, the term “FGF-20” refers to a protein comprising an amino acid sequence of SEQ ID NO:2, or a nucleic acid sequence encoding such a protein and/or a complimentary strand thereof.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90%, 95% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non limiting example, stringent hybridization conditions comprise a salt concentration from about 0.1 M to about 1.0 M sodium ion, a pH from about 7.0 to about 8.3, a temperature is at least about 60° C., and at least one wash in 0.2×SSC, 0.01% BSA. In another non-limiting example, stringent hybridization conditions are hybridization at 6× sodium chloride/sodium citrate (SSC) at about 45° C., followed by one or more washes in 0.1×SSC, 0.2% SDS at about 68° C. In yet another non-limiting example, stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “isolated” in the context of a protein agent refers to a protein agent that is substantially free of cellular material or contaminating proteins from the cell or tissue source from which it is derived, or substantially free of chemical precursors or other chemicals when chemically synthesized. The language “substantially free of cellular material” includes preparations of a protein agent in which the protein agent is separated from cellular components of the cells from which it is isolated or recombinantly produced. Thus, a protein agent that is substantially free of cellular material includes preparations of a protein agent having less than about 30%, 20%, 10%, or 5% (by dry weight) of host cell proteins (also referred to as a “contaminating proteins”). When the protein agent is recombinantly produced, it is also preferably substantially free of culture medium, i.e., culture medium represents less than about 20%, 10%, or 5% of the volume of the protein agent preparation. When the protein agent is produced by chemical synthesis, it is preferably substantially free of chemical precursors or other chemicals, i.e., it is separated from chemical precursors or other chemicals that are involved in the synthesis of the protein agent. Accordingly, such preparations of a protein agent have less than about 30%, 20%, 10%, 5% (by dry weight) of chemical precursors or compounds other than the protein agent of interest. In a specific embodiment, protein agents disclosed herein are isolated.

As used herein, the term “isolated” in the context of nucleic acid molecules refers to a nucleic acid molecule that is separated from other nucleic acid molecules which are present in the natural source of the nucleic acid molecule. Moreover, an “isolated” nucleic acid molecule, such as a cDNA molecule, can be substantially free of other cellular material or culture medium when produced by recombinant techniques, or substantially free of chemical precursors or other chemicals when chemically synthesized. In a specific embodiment, nucleic acid molecules are isolated.

As used herein, the term “in combination” refers to the use of more than one therapy. The use of the term “in combination” does not restrict the order in which therapies are administered to a subject in need thereof. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject in need thereof.

As used herein, the terms “prevent,” “preventing,” and “prevention” refer to the prevention of the recurrence, onset, or development of a disorder associated with an insult affecting a rapidly proliferating tissue or one or more symptoms thereof in a subject resulting from the administration of a therapy (e.g., a composition comprising a CG53135 protein), or the administration of a combination of therapies.

As used herein, the term “prophylactically effective amount” refers to the amount of a therapy (e.g., a composition comprising one or more CG53135 proteins) which is sufficient to result in the prevention of the development, recurrence, or onset of a disease or disorder associated with an insult to rapidly proliferating tissues (such as radiation, chemotherapy, and chemical/biological warfare agents) or one or more symptoms thereof, or to enhance or improve the prophylactic effect(s) of another therapy.

As used herein, the terms “subject” and “subjects” refer to an animal, preferably a mammal, including a non-primate (e.g., a cow, pig, horse, cat, or dog), a primate (e.g., a monkey, chimpanzee, or human), and more preferably a human. In a certain embodiment, the subject is a mammal, preferably a human, who has been exposed to or is going to be exposed to an insult affecting rapidly proliferating tissues (such as radiation, chemotherapy, or chemical/biological warfare agents). In another embodiment, the subject is a farm animal (e.g., a horse, pig, or cow) or a pet (e.g., a dog or cat) which has been exposed to or is going to be exposed to a similar insult. The term “subject” is used interchangeably with “patient” in the present invention.

As used herein, the terms “treat,” “treatment,” and “treating” refer to the reduction of the progression, severity, and/or duration of a disorder associated with an insult affecting a rapidly proliferating tissue or amelioration of one or more symptoms thereof, wherein such reduction and/or amelioration result from the administration of one or more therapies (e.g., a composition comprising a CG53135 protein).

As used herein, the term “therapeutically effective amount” refers to the amount of a therapy (e.g., a composition comprising one or more 53135 proteins) that is sufficient to reduce the severity of a disease or disorder characterized by an insult to rapidly proliferating tissues (such as radiation, chemotherapy, and chemical/biological warfare agents), reduce the duration of such a disease or disorder, prevent the advancement of such a disease or disorder, cause regression of such a disease or disorder, ameliorate one or more symptoms associated with an insult to rapidly proliferating tissues (such as radiation, chemotherapy, and chemical/biological warfare agents), or enhance or improve the therapeutic effect(s) of another therapy.

### 4. BRIEF DESCRIPTION OF THE FIGURES

FIG. 1 shows Liquid Chromatography and Mass Spectrometry analysis of CG53135-05 E. coli purified product.

FIG. 2 depicts tryptic map of CG53135-05 E. coli purified product.

FIG. 3 shows the effect of CG53135 on mouse survival after single exposure to acute radiation dose of 600 cGy.

FIG. 4 shows the mean weight change in mice after single exposure to acute radiation of 600 cGy.

FIG. 5 shows the effect of Phosphate Buffered Saline (PBS) control on mice survival after exposure to radiation doses of 484 cGy, 534 cGy, 570 cGy, 606 cGy, or 641 cGy.

FIG. 6(A) shows the effect of prophylactic administration of CG53135 (day-1) on survival of mice after exposure to radiation doses of 484 cGy, 534 cGy, 570 cGy, 606 cGy, or 641 cGy. (B) shows Kaplan-Meier plots for survival at 570 cGy and 606 cGy, with statistically significant differences between CG53135-treated and PBS-treated control animals. (C) Probit analysis for survival over the range of radiation doses.

FIG. 7 shows the effect of prophylactic administration of CG53135 (day-2 and -1) on survival of mice after exposure to radiation doses of 484 cGy, 534 cGy, 570 cGy, 606 cGy, or 641 cGy.

FIG. 8 shows the cell positions in the crypt. The bottom of the crypt is cell position 1, the crypt base. In the small intestine, the stem cells are located around cell position 4 and the proliferative cells occupy roughly half of the crypt. The cells are constantly maturing such that the cells are fully differentiated and not cycling at the top of the crypt. Changes that may affect stem cells versus their transit amplifying daughter cells can be detected by examining changes in event (labeling, apoptosis, mitosis, etc.) frequency at each cell position.

FIG. 9 shows a survival curve of intestinal crypt cells from mice prophylactically administered CG53135 or PBS, following different radiation dosages.

FIG. 10 shows the effect of prophylactic administration of CG53135 on mice intestinal crypt survival after radiation insult.

FIG. 11 shows the effect of CG53135 multiple-dose administration prior to irradiation on crypt survival curves. Animals (n=6/group) were administered PBS or CG53135-05 E. coli purified product (12 mg/kg) by intraperitoneal (IP) injection once daily for 4 consecutive days prior to a single 10, 11, 12, 13, or 14 Gy dose of X-ray whole-body irradiation on Day 0. The plot represents the radiation dose-response for crypt survival. Data points represent crypt survival in individual animals analyzed using a multi-target (Puck) analysis model, DRFIT.

FIG. 12 shows effect of CG53135 multiple-dose administration on crypt survival curves. Animals (n=6/group) were administered PBS or CG53135-05 E. coli (4 mg/kg) by intraperitoneal (IP) injection once daily for either for 1, 2, 3, 4 or 5 consecutive days prior to, or post a sigle dose (13 Gy) whole body irradiation on Day 0. The plot represents the level of protection of crypt cells in response to treatment schedule. Protection factor value indicates the number of surviving crypts per circumference in the CG53135-05-treated animals compared to PBS, expressed as a ratio.

FIGS. 13A and 13B show CG53135 induced expression of scavengers, cycloxegenase, trefoil factor, and transcription factors in NIH 3T3 cells.

FIG. 14 shows CG53135 induced expression of scavengers, cycloxegenase, trefoil factor, and transcription factors in CCD1070sk cells.

FIG. 15(A) shows CG53135 induced expression of scavengers, cycloxegenase, trefoil factor, and transcription factors in CCD18Co cells. (B) shows activation of ERK and AKT kinases by CG53135.

FIG. 16 shows CG53135 induced expression of scavengers, cycloxegenase, trefoil factor, and transcription factors in human umbilical vein endothelial cells (HUVEC).

FIG. 17 shows the effect of CG53135 on the survival of IEC 18 cells irradiated with different X-ray doses.

FIG. 18 shows the effect of CG53135 on the survival of NIH 3T3 cells irradiated with different X-ray doses.

FIG. 19 shows the effect of CG53135 on the survival of HUVEC irradiated with different X-ray doses.

FIG. 20 shows survival curves for irradiated cells. Cells of various types (hematopoietic—32D; mesenchymal—CCD18-Co and NIH3T3; epithelial—IEC18, IEC6 and bone—U2OS and Saos-2) were irradiated at the indicated doses then plated in complete growth media either with or without (untreated) 100 ng/ml CG53135-05 E. coli purified product and allowed to form colonies for 10-14 days until the colonies grew to an average diameter of 2 mm. The colonies were stained with crystal violet and counted. The natural log (Ln) of the surviving fraction is represented on the Y axis, and bars represent standard error.

FIG. 21(A) shows the effect of CG53135 on the release of cytokine in NIH 3T3 cells. (B) shows IL-6 and IL-11 expression in response to CG53135.

FIG. 22 shows dose response of CM-H₂DCFDA fluorescence from IEC18 cells treated with CG53135 after 4 Gy irradiation.

FIG. 23 shows response of CM-H₂DCFDA fluorescence from IEC18 cells treated with CG53135 after 2 Gy and 4 Gy irradiation.

FIG. 24 shows dose response of CM-H₂DCFDA fluorescence from CCD-18Co cells treated with CG53135 after 4 Gy irradiation.

FIG. 25 shows dose response of Red CC-1 fluorescence from IEC18 cells treated with CG53135 after 4 Gy irradiation.

FIG. 26 shows response of Red CC-1 fluorescence from IEC18 cells treated with CG53135 after 4 Gy and 6 Gy irradiation.

FIG. 27 shows response of Red CC-1 fluorescence from CCD-18Co cells treated with CG53135 before and after 10 Gy irradiation.

FIG. 28 shows in vitro radioprotection of the myeloid cell line 32D by CG53135.

FIG. 29 shows effect of CG53135 on repopulation of thymus following bone marrow ablation and subsequent bone marrow transplant.

FIGS. 30(A) and (B) show effects of CG53135 on body weight in animals with gastrointestinal injury induced by whole body irradiation as analyzed by one-way ANOVA and Dunnett's Multiple Comparison Test, respectively.

FIGS. 31(A) and (B) show effects of CG53135 on diarrhea score in mice with gastrointestinal injury induced by whole body irradiation as analyzed by one-way ANOVA and Tukey's Multiple Comparison Test, respectively.

FIG. 32 shows analysis of diarrhea score for each day of observation.

### 5. DETAILED DESCRIPTION OF THE INVENTION

For clarity of disclosure, and not by way of limitation, the detailed description of the invention is divided into the following subsections:
(i) CG53135
(ii) Methods of Preparing CG53135
(iii) Prophylactic and Therapeutic Uses of CG53135
(iv) Administration and Pharmaceutical Compositions

5.1. CG53135

The present invention provides methods of stimulating stem cell proliferation and/or engraftment and methods of preventing and/or treating disorders associated with one or more insults affecting rapidly proliferating tissues in a body (e.g., radiation exposure, chemical and/or biological insults) or one or more symptoms thereof by administering to a subject a composition comprising one or more CG53135 proteins. As used herein, the term “CG53135” refers to a class of proteins (including peptides and polypeptides) or nucleic acids encoding such proteins or their complementary strands, where the proteins comprise an amino acid sequence of SEQ ID NO:2 (211 amino acids), or its fragments, derivatives, variants, homologs, or analogs.

In one embodiment, a CG53135 protein is a variant of FGF-20. It will be appreciated by those skilled in the art that DNA sequence polymorphisms that lead to changes in the amino acid sequences of the FGF-20 protein may exist within a population (e.g., the human population). Such genetic polymorphism in the FGF-20 gene may exist among individuals within a population due to natural allelic variation. Such natural allelic variations can typically result in 1-5% variance in the nucleotide sequence of the FGF-20 gene. Any and all such nucleotide variations and resulting amino acid polymorphisms in the FGF-20 protein, which are the result of natural allelic variation of the FGF-20 protein, are intended to be within the scope of the invention. In one embodiment, a CG53135 is CG53135-12 (SEQ ID NOs:21 and 22), which is a single nucleotide polymorphism (“SNP”) of FGF-20 (i.e., ²⁰⁶D? N). (For more detailed description of CG53135-12, see e.g., U.S. patent application Ser. No. 10/702,126, filed Nov. 4, 2003, the disclosure of which is incorporated herein by reference in its entirety.) Additional examples of FGF-20 SNPs can be found in Example 2 of U.S. patent application Ser. No. 10/435,087, filed May 9, 2003, the content of which is incorporated by reference by its entirety.

In another embodiment, CG53135 refers to a nucleic acid molecule encoding a FGF-20 protein from other species or the protein encoded thereby, and thus has a nucleotide or amino acid sequence that differs from the human sequence of FGF-20. Nucleic acid molecules corresponding to natural allelic variants and homologues of the FGF-20 cDNAs of the invention can be isolated based on their homology to the human FGF-20 nucleic acids disclosed herein using the human cDNAs, or a portion thereof, as a hybridization probe according to standard hybridization techniques under stringent hybridization conditions.

In another embodiment, CG53135 refers to a fragment of an FGF-20 protein, including fragments of variant FGF-20 proteins, mature FGF-20 proteins, and variants of mature FGF-20 proteins, as well as FGF-20 proteins encoded by allelic variants and single nucleotide polymorphisms of FGF-20 nucleic acids. An example of an FGF-20 protein fragment includes, but is not limited to, residues 2-211, 3-211, 9-211, 12-211, 15-211, 24-211, 54-211, or 55-211 of FGF-20 (SEQ ID NO:2). In one embodiment, CG53135 refers to a nucleic acid encodes a protein fragment that includes residues 2-211, 3-211, 9-211, 12-211, 15-211, 24-211, 54-211, or 55-211 of SEQ ID NO:2.

The invention also encompasses derivatives and analogs of FGF-20. The production and use of derivatives and analogs related to FGF-20 are within the scope of the present invention.

In a specific embodiment, the derivative or analog is functionally active, i.e., capable of exhibiting one or more functional activities associated with a full-length, wild-type FGF-20. Derivatives or analogs of FGF-20 can be tested for the desired activity by procedures known in the art, including but not limited to, using appropriate cell lines, animal models, and clinical trials.

In particular, FGF-20 derivatives can be made via altering FGF-20 sequences by substitutions, insertions or deletions that provide for functionally equivalent molecules. In one embodiment, such alteration of an FGF-20 sequence is done in a region that is not conserved in the FGF protein family. Due to the degeneracy of nucleotide coding sequences, other DNA sequences which encode substantially the same amino acid sequence as FGF-20 may be used in the practice of the present invention. These include, but are not limited to, nucleic acid sequences comprising all or portions of FGF-20 that are altered by the substitution of different codons that encode a functionally equivalent amino acid residue within the sequence, thus producing a silent change. In a preferred embodiment, a wild-type FGF-20 nucleic acid sequence is codon-optimized to the nucleic acid sequence of SEQ ID NO:8 (CG53135-05). Likewise, the FGF-20 derivatives of the invention include, but are not limited to, those containing, as a primary amino acid sequence, all or part of the amino acid sequence of FGF-20 including altered sequences in which functionally equivalent amino acid residues are substituted for residues within the sequence resulting in a silent change. For example, one or more amino acid residues within the sequence can be substituted by another amino acid of a similar polarity that acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. FGF-20 derivatives of the invention also include, but are not limited to, those containing, as a primary amino acid sequence, all or part of the amino acid sequence of FGF-20 including altered sequences in which amino acid residues are substituted for residues with similar chemical properties. In a specific embodiment, 1, 2, 3, 4, or 5 amino acids are substituted.

Derivatives or analogs of FGF-20 include, but are not limited to, those proteins which are substantially homologous to FGF-20 or fragments thereof, or whose encoding nucleic acid is capable of hybridizing to the FGF-20 nucleic acid sequence.

The FGF-20 derivatives and analogs of the invention can be produced by various methods known in the art. The manipulations that result in their production can occur at the gene or protein level. For example, the cloned FGF-20 gene sequence can be modified by any of numerous strategies known in the art (e.g., Maniatis, T., 1989, Molecular Cloning, A Laboratory Manual, 2d ed., Cold Spring Harbor Laboratory, Cold Spring Harbor, N.Y.). The sequence can be cleaved at appropriate sites with restriction endonuclease(s), followed by further enzymatic modification if desired, isolated, and ligated in vitro. In the production of the gene encoding a derivative or analog of FGF-20, care should be taken to ensure that the modified gene remains within the same translational reading frame as FGF-20, uninterrupted by translational stop signals, in the gene region where the desired FGF-20 activity is encoded.

Additionally, the FGF-20-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including but not limited to, in vitro site-directed mutagenesis (Hutchinson, C. et al., 1978, J. Biol. Chem 253:6551), use of TAB.RTM. linkers (Pharmacia), etc.

Manipulations of the FGF-20 sequence may also be made at the protein level. Included within the scope of the invention are FGF-20 fragments or other derivatives or analogs which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, reagents useful for protection or modification of free NH2-groups, free COOH-groups, OH-groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH4; acetylation, formylation, oxidation, reduction; metabolic synthesis in the presence of tunicamycin; etc.

In addition, analogs and derivatives of FGF-20 can be chemically synthesized. For example, a protein corresponding to a portion of FGF-20 which comprises the desired domain, or which mediates the desired aggregation activity in vitro, or binding to a receptor, can be synthesized by use of a peptide synthesizer. Furthermore, if desired, nonclassical amino acids or chemical amino acid analogs can be introduced as a substitution or addition into the FGF-20 sequence. Non-classical amino acids include, but are not limited to, the D-isomers of the common amino acids, a-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Ca-methyl amino acids, and Na-methyl amino acids.

In a specific embodiment, the FGF-20 derivative is a chimeric or fusion protein comprising FGF-20 or a fragment thereof fused via a peptide bond at its amino- and/or carboxy-terminus to a non-FGF-20 amino acid sequence. In one embodiment, the non-FGF-20 amino acid sequence is fused at the amino-terminus of an FGF-20 or a fragment thereof. In another embodiment, such a chimeric protein is produced by recombinant expression of a nucleic acid encoding the protein (comprising an FGF-20-coding sequence joined in-frame to a non-FGF-20 coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, a chimeric nucleic acid encoding FGF-20 with a heterologous signal sequence is expressed such that the chimeric protein is expressed and processed by the cell to the mature FGF-20 protein. The primary sequence of FGF-20 and non-FGF-20 gene may also be used to predict tertiary structure of the molecules using computer simulation (Hopp and Woods, 1981, Proc. Natl. Acad. Sci. U.S.A. 78:3824-3828); the chimeric recombinant genes could be designed in light of correlations between tertiary structure and biological function. Likewise, chimeric genes comprising an essential portion of FGF-20 molecule fused to a heterologous (non-FGF-20) protein-encoding sequence may be constructed. In a specific embodiment, such chimeric construction can be used to enhance one or more desired properties of an FGF-20, including but not limited to, FGF-20 stability, solubility, or resistance to proteases. In another embodiment, chimeric construction can be used to target FGF-20 to a specific site. In yet another embodiment, chimeric construction can be used to identify or purify an FGF-20 of the invention, such as a His-tag, a FLAG tag, a green fluorescence protein (GFP), β-galactosidase, a maltose binding protein (MalE), a cellulose binding protein (CenA) or a mannose protein, etc. In one embodiment, a CG53135 protein is carbamylated.

In some embodiment, a CG53135 protein can be modified so that it has an extended half-life in vivo using any methods known in the art. For example, inert polymer molecules such as high molecular weight polyethyleneglycol (PEG) can be attached to a CG53135 protein with or without a multifunctional linker either through site-specific conjugation of the PEG to the N- or C-terminus of the protein or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the CG53135 protein. Unreacted PEG can be separated from CG53135-PEG conjugates by size-exclusion or by ion-exchange chromatography. PEG-derivatized conjugates can be tested for in vivo efficacy using methods known to those of skill in the art.

A CG53135 protein can also be conjugated to albumin in order to make the protein more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

In some embodiments, CG53135 refers to CG53135-01 (SEQ ID NOs:1 and 2), CG53135-02 (SEQ ID NOs:3 and 4), CG53135-03 (SEQ ID NOs:5 and 2), CG53135-04 (SEQ ID NOs:6 and 7), CG53135-05 (SEQ ID NOs:8 and 2), CG53135-06 (SEQ ID NOs:9 and 10), CG53135-07 (SEQ ID NOs:11 and 12), CG53135-08 (SEQ ID NOs:13 and 14), CG53135-09 (SEQ ID NOs:15 and 16), CG53135-10 (SEQ ID NOs:17 and 18), CG53135-11 (SEQ ID NOs:19 and 20), CG53135-12 (SEQ ID NOs:21 and 22), CG53135-13 (SEQ ID NOs:23 and 24), CG53135-14 (SEQ ID NOs:25 and 26), CG53135-15 (SEQ ID NOs:27 and 28), CG53135-16 (SEQ ID NOs:29 and 30), CG53135-17 (SEQ ID NOs:31 and 32), IFC 250059629 (SEQ ID NOs:33 and 34), IFC 20059669 (SEQ ID NOs:35 and 36), IFC 317459553 (SEQ ID NOs:37 and 38), IFC 317459571 (SEQ ID NOs:39 and 40), IFC 250059596 (SEQ ID NOs:41 and 10), IFC316351224 (SEQ ID NOs:41 and 10), or a combination thereof. In a specific embodiment, a CG53135 is carbamylated, for example, a carbamylated CG53135-13 protein or a carbamylated CG53135-05 protein.

5.2. Methods of Preparing CG53135

Examples of methods of isolating a CG53135 protein are described in Section 6, infra, as well as in previously filed applications, e.g., U.S. patent application Ser. No. 09/609,543, filed Jul. 3, 2000, and Ser. No. 10/174,394, filed Jun. 17, 2002, both of which are incorporated herein by reference. Any techniques known in the art can be used in purifying a CG53135 protein, including but not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See e.g., Scopes, PROTEIN PURIFICATION, PRINCIPLES AND PRACTICE, 3rd ed., Springer (1994). During the purification, the biological activity of CG53135 may be monitored by one or more in vitro or in vivo assays. The purity of CG53135 can be assayed by any methods known in the art, such as but not limited to, gel electrophoresis. See Scopes, supra. In some embodiment, the CG53135 proteins employed in a composition of the invention can be in the range of 80 to 100 percent of the total mg protein, or at least 80%, at least 85%, at least 90%, at least 95%, or at least 98% of the total mg protein. In one embodiment, one or more CG53135 proteins employed in a composition of the invention is at least 99% of the total protein. In another embodiment, CG53135 is purified to apparent homogeneity, as assayed, e.g., by sodium dodecyl sulfate polyacrylamide gel electrophoresis.

Methods known in the art can be utilized to recombinantly produce CG53135 proteins. A nucleic acid sequence encoding a CG53135 protein can be inserted into an expression vector for propagation and expression in host cells.

An expression construct, as used herein, refers to a nucleic acid sequence encoding a CG53135 protein operably associated with one or more regulatory regions that enable expression of a CG53135 protein in an appropriate host cell. “Operably-associated” refers to an association in which the regulatory regions and the CG53135 sequence to be expressed are joined and positioned in such a way as to permit transcription, and ultimately, translation.

The regulatory regions necessary for transcription of CG53135 can be provided by the expression vector. A translation initiation codon (ATG) may also be provided if a CG53135 gene sequence lacking its cognate initiation codon is to be expressed. In a compatible host-construct system, cellular transcriptional factors, such as RNA polymerase, will bind to the regulatory regions on the expression construct to effect transcription of the modified CG53135 sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to a CG53135 gene sequence or to insert a CG53135 gene sequence into the cloning site of a vector, linkers or adapters providing the appropriate compatible restriction sites may be ligated to the ends of the cDNAs by techniques well known in the art (see e.g., Wu et al., 1987, Methods in Enzymol, 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA using PCR with primers containing the desired restriction enzyme site.

An expression construct comprising a CG53135 sequence operably associated with regulatory regions can be directly introduced into appropriate host cells for expression and production of a CG53135 protein without further cloning. See, e.g., U.S. Pat. No. 5,580,859. The expression constructs can also contain DNA sequences that facilitate integration of a CG53135 sequence into the genome of the host cell, e.g., via homologous recombination. In this instance, it is not necessary to employ an expression vector comprising a replication origin suitable for appropriate host cells in order to propagate and express CG53135 in the host cells.

A variety of expression vectors may be used, including but are not limited to, plasmids, cosmids, phage, phagemids or modified viruses. Such host-expression systems represent vehicles by which the coding sequences of a CG53135 gene may be produced and subsequently purified, but also represent cells which may, when transformed or transfected with the appropriate nucleotide coding sequences, express CG53135 in situ. These include, but are not limited to, microorganisms such as bacteria (e.g., E. coli and B. subtilis) transformed with recombinant bacteriophage DNA, plasmid DNA or cosmid DNA expression vectors containing CG53135 coding sequences; yeast (e.g., Saccharomyces, Pichia) transformed with recombinant yeast expression vectors containing CG53135 coding sequences; insect cell systems infected with recombinant virus expression vectors (e.g., baculovirus) containing CG53135 coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing CG53135 coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells are used for the expression of a recombinant CG53135 molecule. For example, mammalian cells such as Chinese hamster ovary cells (CHO) can be used with a vector bearing promoter element from major intermediate early gene of cytomegalocirus for effective expression of a CG53135 sequence (Foecking et al., 1986, Gene 45:101; and Cockett et al., 1990, Bio/Technology 8:2).

In bacterial systems, a number of expression vectors may be advantageously selected depending upon the use intended for the CG53135 molecule being expressed. For example, when a large quantity of a CG53135 is to be produced, for the generation of pharmaceutical compositions of a CG53135 molecule, vectors that direct the expression of high levels of readily purified fusion protein products may be desirable. Such vectors include, but are not limited to, the E. coli expression vector pCR2.1 TOPO (Invitrogen); pIN vectors (Inouye & Inouye, 1985, Nucleic Acids Res. 13:3101-3109; Van Heeke & Schuster, 1989, J. Biol. Chem. 24:5503-5509) and the like. Series of vectors like pFLAG (Sigma), pMAL (NEB), and pET (Novagen) may also be used to express the foreign proteins as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific proteases like enterokinase allows the CG53135 protein to be cleaved from the fusion protein. The pGEX vectors may also be used to express foreign proteins as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. A CG53135 coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, a CG53135 coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing CG53135 in infected hosts (see, e.g., Logan & Shenk, 1984, Proc. Natl. Acad. Sci. USA 8 1:355-359). Specific initiation signals may also be required for efficient translation of inserted CG53135 coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, W138, BT483, Hs578T, HTB2, BT20 and T47D, NSO (a murine myeloma cell line that does not endogenously produce any immunoglobulin chains), CRL7030 and HsS78Bst cells. Expression in a bacterial or yeast system can be used if post-translational modifications turn to be non-essential for a desired activity of CG53135. In a preferred embodiment, E. coli is used to express a CG53135 sequence.

For long-term, high-yield production of properly processed CG53135, stable expression in cells is preferred. Cell lines that stably express CG53135 may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while CG53135 is expressed continuously.

A number of selection systems may be used, including but not limited to, antibiotic resistance (markers like Neo, which confers resistance to geneticine, or G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):I55-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); and hygro, which confers resistance to hygromycin (Santerre et al., 1984, Gene 30:147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of CG53135. Modified culture conditions and media may also be used to enhance production of CG53135. Any techniques known in the art may be applied to establish the optimal conditions for producing CG53135.

An alternative to producing CG53135 or a fragment thereof by recombinant techniques is peptide synthesis. For example, an entire CG53135, or a protein corresponding to a portion of CG53135, can be synthesized by use of a peptide synthesizer. Conventional peptide synthesis or other synthetic protocols well known in the art may be used.

Proteins having the amino acid sequence of CG53135 or a portion thereof may be synthesized by solid-phase peptide synthesis using procedures similar to those described by Merrifield, 1963, J. Am. Chem. Soc., 85:2149. During synthesis, N-a-protected amino acids having protected side chains are added stepwise to a growing polypeptide chain linked by its C-terminal and to an insoluble polymeric support, i.e., polystyrene beads. The proteins are synthesized by linking an amino group of an N-a-deprotected amino acid to an a-carboxyl group of an N-a-protected amino acid that has been activated by reacting it with a reagent such as dicyclohexylcarbodiimide. The attachment of a free amino group to the activated carboxyl leads to peptide bond formation. The most commonly used N-a-protecting groups include Boc, which is acid-labile, and Fmoc, which is base-labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CG53135 is accomplished using conventional procedures, such as preparative HPLC using gel permeation, partition and/or ion exchange chromatography. The choice of appropriate matrices and buffers are well known in the art and so are not described in detail herein.

Non-limiting examples of methods for preparing CG53135 can be found in Section 6, infra.

5.3. Prophylactic and Therapeutic Uses of CG53135

The present invention provides methods of preventing and/or treating one or more disorders associated with (e.g., caused by) radiation exposure, chemotherapy, chemical/biological warfare agents, and/or any other insults affecting rapidly proliferating tissues in the body, or one or more symptoms thereof by administering to a subject a prophylactically or therapeutically effective amount of a composition comprising one or more isolated CG53135 proteins.

In one embodiment, the present invention provides methods of preventing and/or treating a pathology of epithelial cells and/or mesenchymal cells comprising administering to a subject in need thereof a composition comprising one or more CG53135 proteins. In another embodiment, the present invention provides methods of stimulating proliferation, differentiation or migration of epithelial cells and/or mesenchymal cells comprising administering to a subject in need thereof an effective amount of a composition comprising one or more CG53135 proteins.

Epithelial membranes are continuous sheets of cells with contiguous cell borders that have characteristic specialized sites of close contact called cell junction. Such membrane, which can be one or more cells thick, contain no capillaries. Epithelia are attached to the underlying connective tissue by a component known as a basement membrane, which is a layer of intercellular material of complex composition that is distributed as a thin layer between the epithelium and the connective tissue.

Stratified squamous nonkeratinizing epithelium is common on wet surfaces that are subject to considerable wear and tear at sites where absorptive function is not required. The secretions necessary to keep such surfaces wet have to come from appropriately situated glands. Sites lined by this type of epithelium include the esophagus and the floor and sides of the oral cavity.

Simple columnar epithelium is made up of a single layer of tall cells that again fit together in a hexagonal pattern. In simple secretory columnar epithelium, the columnar cells are all specialized to secret mucus in addition to being protective. Sites of this type of epithelium is present include the lining of the stomach.

A simple columnar epithelium that is made up of absorptive cells as well as secretory cells lines the intestine. To facilitate absorption, this membrane is only one cell thick. Interspersed with cells that are specialized for absorption, there are many goblet cells that secrete protective mucus.

Mesenchymal cells are stem cells that can differentiate into, e.g., osteoblasts, chondrocytes, myocytes, and adipocytes. Mesenchymal-epithelial interactions play an important role in the physiology and pathology of epithelial tissues. Messenchymal cells may associate with epithelium basement membrane (e.g., pericytes and perivascular monocyte-derived cells (MDCs)), or reside within epithelium (MDCs and T cells). The nature of the interactions between mesenchymal cells and tissue-specific cells may depend on the tissue type (e.g., brain versus epidermis), or on the prevention or allowance/stimulation of differentiation of cells into the suicidal state (apoptosis) by mesenchymal cells in a given epithelium. Specialized mesenchymal cells, such as pericytes, MDCs, and T lymphocytes, may significantly influence the differentiation and aging of epithelial cells.

The stromal compartment of the cavities of bone is composed of a net-like structure of interconnected mesenchymal cells. Stromal cells are closely associated with bone cortex, bone trabecule and to the hemopoietic cells. The bone mmarrow-stromal micro-environment, is a complex of cells, extracellular matrix (ECM) with growth factors and cytokines that regulate osteogenesis and hemopoiesis locally throughout the life of the individual. The role of the marrow stroma in creating the microenvironment for bone physiology and hemopoiesis lies in a specific subpopulation of the stroma cells. They differentiate from a common stem cell to the specific lineage each of which has a different role. Their combined function results in orchestration of a 3-D-architecture that maintains the active bone marrow within the bone.

In adults, blood cells are produced by the bone marrow, the spongy material filling the body's bones. The bone marrow produces two blood cell groups, myeloid and lymphoid. The myeloid cell line includes, e.g., the following: (1) Immature cells called erythrocytes that later develop into red blood cells; (2) Blood clotting agents (platelets); (3) Some white blood cells, including macrophages (which act as scavengers for foreign particles), eosinophils (which trigger allergies and also defend against parasites), and neutrophils (the main defenders against bacterial infections). The lymphoid cell line includes, e.g., the lymphocytes, which are the body's primary infection fighters. Among other vital functions, certain lymphocytes are responsible for producing antibodies, factors that can target and attack specific foreign agents (antigens). Lymphocytes develop in the thymus gland or bone marrow and are therefore categorized as either B-cells (bone marrow-derived cells) or T-cells (thymus gland-derived cells).

According to the present invention, CG53135 can regulate proliferation, differentiation, and/or migration of epithelial cells and/or mesenchymal cells, and thus have prophylactic and/or therapeutic effects on a disorder associated with a pathology of epithelial cells and/or mesenchymal cells.

In some embodiments, a composition used in accordance to the methods of the invention comprises a FGF-20 protein, a fragment, a derivative, a variant, a homolog, or an analog of FGF-20, or a combination thereof. In some embodiments, a composition used in accordance to the methods of the invention comprises CG53135-01 (SEQ ID NO:2), CG53135-02 (SEQ ID NO: 4), CG53135-03 (SEQ ID NO:2), CG53135-04 (SEQ ID NO:7), CG53135-05 (SEQ ID NO: 2), CG53135-06 (SEQ ID NO: 10), CG53135-07 (SEQ ID NO:12), CG53135-08 (SEQ ID NO:14), CG53135-09 (SEQ ID NO:16), CG53135-10 (SEQ ID NO:18), CG53135-11 (SEQ ID NO:20), CG53135-12 (SEQ ID NO:22), CG53135-13 (SEQ ID NO:24), CG53135-14 (SEQ ID NO:26), CG53135-15 (SEQ ID NO:28), CG53135-16 (SEQ ID NO:30), CG53135-17 (SEQ ID NO:32), IFC 250059629 (SEQ ID NO:34), IFC 20059669 (SEQ ID NO:36), IFC 317459553 (SEQ ID NO:38), IFC 317459571 (SEQ ID NO:40), IFC 250059596 (SEQ ID NO:10), or IFC316351224 (SEQ ID NO:10), or any two or more combinations of CG53135 proteins. In one embodiment, the compositions used in accordance to the methods of the present invention comprise (1) a protein comprising an amino acid sequence of SEQ ID NO:2, and (2) a protein comprising an amino acid sequence of SEQ ID NO:24. In another embodiment, the compositions used in accordance to the methods of the present invention comprise (1) a protein comprising an amino acid sequence of SEQ ID NO:2, (2) a protein comprising an amino acid sequence of SEQ ID NO:24, (3) a protein comprising an amino acid sequence of SEQ ID NO:26, (4) a protein comprising an amino acid sequence of SEQ ID NO:28, (5) a protein comprising an amino acid sequence of SEQ ID NO:30, and (6) a protein comprising an amino acid sequence of SEQ ID NO:32. In another embodiment, the compositions used in accordance to the methods of the present invention comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, and (5) a protein comprising an amino acid sequence of SEQ ID NO:32. In another embodiment, the compositions used in accordance to the methods of the present invention comprise (1) a protein comprising an amino acid sequence of SEQ ID NO:32; (2) a protein comprising an amino acid sequence of SEQ ID NO:30, (3) a protein comprising an amino acid sequence of SEQ ID NO:28; and (4) a protein comprising an amino acid sequence of SEQ ID NO:24. In yet another embodiment, the compositions used in accordance to the methods of the present invention comprise (1) a protein comprising an amino acid sequence of SEQ ID NO:2, (2) a protein comprising an amino acid sequence of SEQ ID NO:24, (3) a protein comprising an amino acid sequence of SEQ ID NO:28, (4) a protein comprising an amino acid sequence of SEQ ID NO:30, (5) a protein comprising an amino acid sequence of SEQ ID NO:32, (6) a carbamylated protein comprising an amino acid sequence of SEQ ID NO:24, and (7) a carbamylated protein comprising an amino acid sequence of SEQ ID NO:2.

In some embodiments, an insult affecting rapidly proliferating tissues is radiation exposure. In a specific embodiment, the insult is ionizing radiation. In another embodiment, the insult may be one or more chemotherapies or one or more chemical/biological warfare agents (such as a vesicant agent or bacteria), or a combination thereof. Non-limiting examples of chemotherapy and chemical/biological warfare agent are alkylating agents, vesicant agents (e.g., mustard agents) and microorganisms. In some embodiments, an insult affecting rapidly proliferating tissues is one or more radiation exposures, one or more chemotherapies, one or more chemical/biological warfare agents, or a combination thereof.

Organs and body systems most sensitive to the effects of insult such as ionizing radiation include, but are not limited to, skin, hematopoietic and lymphatic systems, gonads, lungs, nerve tissues, and the GI tract. In one embodiment, the insult are particularly damaging to hematopoietic and/or gastrointestinal tissues of a subject. In a specific embodiment, the disorder to be prevented or treated is a disorder of hematopoiesis, including but not limited to, anemia, leukopenia (e.g., neutropenia), thrombocytopenia, pancytopenia, and a clotting disorder. In another embodiment, the disorder to be prevented or treated is alimentary mucositis, including but not limited to, oral mucositis, esophagitis, stomatitis, enteritis, and proctitis. In another embodiment, the disorder to be prevented or treated is a cerebrovascular syndrome. In some embodiments, the symptoms associated with an insult affecting rapidly proliferating tissues (such as radiation, chemotherapy, and chemical/biological warfare agents) include, but are not limited to, diarrhea, skin burn, sores, fatigue, dehydration, inflammation, hair loss, ulceration of oral mucosa, xerostomia, and bleeding (e.g., from the nose, mouth or rectum).

The present invention also provides methods of upregulating oxygen scavenging pathways in a subject, where the methods comprise administering to the subject a composition comprising one or more CG53135 proteins. In one embodiment, the oxygen scavenging pathways comprise one or more superoxide dismutases (“SOD”), including but are not limited to, intracellular CuZnSOD and MnSOD, and extracellular-SOD (“EC-SOD”). In another embodiment, the oxygen scavenging pathways comprise genes selected from the group consisting of ERK, AKT, a superoxide dismutase, cyclooxygenase-2 (“COX-2”), and Nrf-2. Cells exposed to radiation must be able to deal with the detrimental effects of ionized radicals (reactive oxygen species or ROS), of which the most reactive species within the cell are generated by the ionization of H₂O. Administering one or more CG53135 proteins to a subject increases transcription of enzymes, like superoxide, that scavenge ROS and convert them to less reactive intermediates, like hydrogen peroxide. Administering one or more CG53135 proteins to a subject also reduces the load of reactive oxygen species induced by an insult, such as radiation.

The present invention further provides methods of stimulating secretion of one or more endogenous cytokines and/or endogenous chemokines from cells of a subject comprising administering to the subject a composition comprising one or more CG53135 proteins. The endogenous cytokines secreted can be, but are not limited to, interleukin (“IL”)-1b, IL-6, IL-7, IL-8, IL-11, and granulocyte-colony forming factor (“G-CSF”). The endogenous chemokines secreted can be, but are not limited to, chemokine (C-X-C motif) ligand 1 (“CXCL1”) and monocyte chemoattractant protein (“MCP-1”). Some of these endogenous cytokines and chemokines have been shown to be involved in endogenous radioprotective responses.

The present invention provides methods of stimulating proliferation of hematopoietic stem cells and/or gastrointestinal stem cells of a subject, where the methods comprise administering to the subject a composition comprising one or more CG53135 proteins. In one embodiment, administration of one or more CG53135 proteins to a subject stimulates fibroblast cells within the bone marrow stroma to secret factors that facilitate the health and proliferative capacity of hematopoietic stem cells. In another embodiment, administration of one or more CG53135 proteins to a subject leads to a rapid proliferative burst of gastrointestinal stem cells, which is followed by a counter-regulatory inhibition in proliferation 24 hours later. This leads to a synchronization of the cell cycle at the tissue level, which is more radio-resistant.

The present invention also provides methods of optimizing engraftment of hematopoietic stem cells in a subject, where the methods comprise administering to the subject a composition comprising one or more CG53135 proteins. In one embodiment, administration of one or more CG53135 proteins improves successful engraftment or repopulation of T-cells following a bone marrow transplant after marrow radioablation. In another embodiment, administration of one or more CG53135 proteins increases the speed of T-cell reconstitution within the thymus after bone marrow transplant.

The patient population that can be targeted using the methods of the present invention include, but are not limited to, subjects who have been exposed to an insult affecting rapidly proliferating tissues (such as radiation, chemotherapy, and chemical/biological warfare agents), subjects who are suspected to have been exposed an insult affecting rapidly proliferating tissues (such as radiation, chemotherapy, and chemical/biological warfare agents), subjects who will be exposed to an insult affecting rapidly proliferating tissues (such as radiation, chemotherapy, and chemical/biological warfare agents), and subjects who are at risk to be exposed to an insult affecting rapidly proliferating tissues (such as radiation, chemotherapy, and chemical/biological warfare agents).

In one embodiment, a composition comprising one or more CG53135 proteins is administered to a subject prior to the subject's exposure to an insult affecting rapidly proliferating tissue in a body. In another embodiment, a composition comprising one or more CG53135 proteins is administered to a subject after the subject's exposure to an insult affecting rapidly proliferating tissue in a body but prior to a disorder associated with the insult or a symptom thereof developed in the subject. In another embodiment, a composition comprising one or more CG53135 proteins is administered to a subject after a disorder associated with an insult affecting rapidly proliferating tissue in a body or a symptom thereof developed in the subject. In yet another embodiment, a composition comprising one or more CG53135 proteins is administered to a subject who is at risk for exposure to an insult affecting rapidly proliferating tissues.

Compositions comprising one or more CG53135 proteins can also be administered in combination with one or more other therapies to prevent, treat, or ameliorate a disorder or one or more symptoms associated with an insult affecting rapidly proliferating tissues (such as radiation, chemotherapy, and chemical/biological warfare agents). In a preferred embodiment, a composition comprising one or more CG53135 proteins is administered in combination with one or more other therapies known to be used in preventing, treating, or ameliorating a disorder or one or more symptoms associated with an insult affecting rapidly proliferating tissues (such as radiation, chemotherapy, and chemical/biological warfare agents). Examples of such other therapies include, but are not limited to, Mesna (sodium 2-mercaptoethene sulfonate) and other analogues with free thiol moieties, dimesna (disodium 2,2′-dithiobis ethane sulfonate) and other disulfides, and compounds such as, for example, described in U.S. Application Publication No. 20030092681, and KGF (see e.g., U.S. Pat. No. 6,743,422). Examples of other agents that can be used in combination with a composition comprising CG53135 is shown in Table 1B.

TABLE 1B

| Trade name, | | | | |
|---|---|---|---|---|
| common or | Company/ | Administration | | |
| chemical name | Supplier | Method(s) | Mechanism | Treatment Mode |
| Amifostine | Medimmune Inc. | IV (200 mg/m²) | Free radical | Possible prophylaxis for |
| Ethyol, WR-2721 | | approved | scavenger | first responders |
| phosphorylated | | SC (500 mg/m²) | Protection from | 15-30 minutes prior to |
| aminothiol | | possible | reactive oxygen | exposure to radiation |
| | | | species | |
| Sodium | Pharmacies | 4 g initial | Alkalinization of | Treatment following |
| bicarbonate | | followed by 2 g | urine leading to | ingestion of uranium |
| | | every 4 h | rapid secretion of | (²³⁵U) |
| | | | uranium | |
| | | | carbonate | |
| | | | complex | |
| Ca-DTPA | HEYL Chemical | 1 g IV or | Metal chelation | Treatment following |
| Zn-DTPA | Pharmaceutical | nebulizer | (Calcium is | ingestion or inhalation |
| calcium or zinc | Factory (Berlin) | | substituted by | of plutonium, |
| salt of diethylene | Distributed by: | | other metals) | americium |
| triamine pentaacetate | Oak Ridge | | Promotes | |
| | Associated | | increased renal | |
| | Universities/ | | clearance | |
| | DOE | | | |
| Potassium iodide | Pharmacies | 16-130 mg oral | Thyroid blocker | Prophylaxis prior to |
| | | depending on | Prevents | exposure, or treatment |
| | | exposure and age | accumulation of | immediately following |
| | | | radioiodines | (hrs) |
| Radiogardase | HEYL Chemical | 1 g orally three | Complex with | Treatment following |
| Prussian Blue | Pharmaceutical | times per day | Cesium (¹³⁷Cs) | ingestion or other |
| ferric | Factory (Berlin) | | or Thallium | internal contamination |
| hexacyanoferrate | Administered by: | | leading to | of the gut |
| | Oak Ridge Inst. | | enhanced | |
| | for Science and | | secretion | |
| | Industry/DOE | | | |
| G-CSF | Amgen Inc. | SC or IV | Stimulates the | Off label use following |
| filgrastim, | | 5 μg/kg/day | proliferation, | radiation exposure to |
| Neupogen | | filgrastim | differentiation, | limit life threatening |
| pegfilgrastim, | | SC 6 mg | and function of | infections |
| Neulasta | | pegfilgrastim | neutrophils | Prophylaxis |
| GM-CSF | Berlex (Schering | SC or IV | Stimulates the | Off label use following |
| sargramostim, | AG) | 250 mg/m²/day | proliferation, | radiation exposure to |
| Leukine | | | differentiation, | limit life threatening |
| | | | and function of | infections |
| | | | neutrophils | Prophylaxis |

In one embodiment, during a combination therapy, a CG53135 protein and/or another therapy are administered in a sub-optimal amount, e.g., an amount that does not manifest detectable therapeutic benefits when administered alone, as determined by methods known in the art. In such methods, co-administration of a CG53135 protein and another therapy results in an overall improvement in effectiveness of treatment.

In one embodiment, a composition comprising one or more CG53135 proteins and one or more other therapies are administered within the same patient visit. In another embodiment, a composition comprising one or more CG53135 proteins is administered prior to the administration of one or more other therapies. In yet another embodiment, a composition comprising one or more CG53135 proteins is administered subsequent to the administration of one or more other therapies. In a specific embodiment, a composition comprising one or more CG53135 proteins and one or more other therapies are cyclically administered to a subject. Cycling therapy involves the administration of a composition comprising one or more CG53135 proteins for a period of time, followed by the administration of one or more other therapies for a period of time and repeating this sequential administration. Cycling therapy can reduce the development of resistance to one or more of the therapies, avoid or reduce the side effects of one of the therapies, and/or improve the efficacy of the treatment.

Toxicity and therapeutic efficacy of a composition of the invention (e.g., a composition comprising one or more CG53135 proteins) can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀/ED₅₀. Compositions that exhibit large therapeutic indices are preferred. While compositions that exhibit toxic side effects may be used, care should be taken to design a delivery system that targets such composition to the site of affected tissue in order to minimize potential damage to uninfected cells and, thereby, reduce side effects.

In one embodiment, the data obtained from the cell culture assays and animal studies can be used in formulating a range of dosage for use in humans. The dosage of complexes lies preferably within a range of circulating concentrations that include the ED₅₀ with little or no toxicity. The dosage may vary within this range depending upon the dosage form employed, the route of administration utilized, the severity of the disease, age and weight of the subject, and other factors normally considered by a medical professional (e.g., a physician). For any composition used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell cultures. Such information can be used to more accurately determine useful doses in humans. Levels in plasma may be measured, for example, by high performance liquid chromatography.

The amount of the composition of the invention which will be effective in the treatment of a particular disorder or condition will depend on the nature of the disorder or condition, and can be determined by standard clinical techniques. The precise dose to be employed in the formulation will also depend on the route of administration, and the seriousness of the disease or disorder, and should be decided according to the judgment of the practitioner and each patient's circumstances.

In one embodiment, the dosage of a composition comprising one or more CG53135 proteins for administration in a human patient provided by the present invention is at least 0.001 mg/kg, at least 0.005 mg/kg, at least 0.01 mg/kg, at least 0.03 mg/kg, at least 0.05 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 25 mg/kg, at least 50 mg/kg, at least 75 mg/kg, or at least 100 mg/kg (as measured by UV assay). In another embodiment, the dosage of a composition comprising one or more CG53135 proteins for administration in a human patient provided by the present invention is between 0.001-100 mg/kg, between 0.001-50 mg/kg, between 0.001-25 mg/kg, between 0.001-10 mg/kg, between 0.005-5 mg/kg, between 0.01-1 mg/kg, between 0.01-0.9 mg/kg, between 0.01-0.8 mg/kg, between 0.01-0.7 mg/kg, between 0.01-0.6 mg/kg, between 0.01-0.5 mg/kg, or between 0.01-0.3 mg/kg (as measured by UV assay).

Protein concentration can be measured by methods known in the art, such as Bradford assay or UV assay, and the concentration may vary depending on what assay is being used. In a non-limiting example, the protein concentration in a pharmaceutical composition of the instant invention is measured by a UV assay that uses a direct measurement of the UV absorption at a wavelength of 280 nm, and calibration with a well characterized reference standard of CG53135 protein (instead of IgG). Test results obtained with this UV method (using CG53135 reference standard) are three times lower than test results for the same sample(s) tested with the Bradford method (using IgG as calibrator). For example, if a dosage of a composition comprising one or more CG53135 proteins for administration in a human patient provided by the present invention is between 0.001-10 mg/kg measured by UV assay, then the dosage is 0.003-30 mg/kg as measured by Bradford assay.

In one embodiment, a composition comprising one or more CG53135 proteins is administered to a subject in a single dose to prevent a disorder associate with an insult affecting rapidly proliferating tissues (such as radiation, chemotherapy, and chemical/biological warfare agents) or ameliorate one or more symptoms thereof. In a specific embodiment, such composition is administered no more than 24 hours, no more than 20 hours, no more than 15 hours, no more than 10 hours, or no more than 5 hours prior to the exposure to the insult. In another embodiment, a composition comprising one or more CG53135 proteins is administered to a subject in two or more doses to prevent and/or treat a disorder associate with an insult affecting rapidly proliferating tissues (such as radiation, chemotherapy, and chemical/biological warfare agents) or ameliorate one or more symptoms thereof. Such a composition is preferably administered to a subject both before exposure to an insult (such as radiation, chemotherapy, and chemical/biological warfare agents) and after exposure to an insult (such as radiation, chemotherapy, and chemical/biological warfare agents). In a specific embodiment, such composition is administered 3 days prior, 2 days prior, 1 day prior to the insult, and on the day of insult, and 1 day after the insult, respectively.

The appropriate and recommended dosages, formulation and routes of administration for treatment modalities such as chemotherapeutic agents, radiation therapy and biological/immunotherapeutic agents such as cytokines are known in the art and described in such literature as the Physician's Desk Reference (58th ed., 2004).

5.4. Administration and Pharmaceutical Compositions

Various delivery systems are known and can be used to administer a composition used in accordance to the methods of the invention. Such delivery systems include, but are not limited to, encapsulation in liposomes, microparticles, microcapsules, expression by recombinant cells, receptor-mediated endocytosis, construction of the nucleic acids of the invention as part of a retroviral or other vectors, etc. Methods of introduction include, but are not limited to, intradermal, intramuscular, intraperitoneal, intrathecal, intracerebroventricular, epidural, intravenous, subcutaneous, intranasal, intratumoral, transdermal, transmucosal, rectal, and oral routes. The compositions used in accordance to the methods of the invention may be administered by any convenient route, for example, by infusion or bolus injection, by absorption through epithelial or mucocutaneous linings (e.g., eye mucosa, oral mucosa, vaginal mucosa, rectal and intestinal mucosa, etc.), and may be administered together with other biologically active agents. Administration can be systemic or local. In a specific embodiment, the present invention comprises using single or double chambered syringes, preferably equipped with a needle-safety device and a sharper needle, that are pre-filled with a composition comprising one or more CG53135 proteins. In one embodiment, dual chambered syringes (e.g., Vetter Lyo-Ject dual-chambered syringe by Vetter Pharmar-Fertigung) are used. Such systems are desirable for lyophilized formulations, and are especially useful in an emergency setting.

In some embodiments, it may be desirable to administer the pharmaceutical compositions of the invention locally to the area in need of treatment. This may be achieved by, for example, local infusion during surgery, or topical application, e.g., in conjunction with a wound dressing after surgery, by injection, by means of a catheter, by means of a suppository, or by means of an implant (said implant being of a porous, non-porous, or gelatinous material, including membranes, such as sialastic membranes, or fibers). In one embodiment, administration can be by direct injection at the site (or former site) of rapidly proliferating tissues that are most sensitive to an insult, such as radiation, chemotherapy, or chemical/biological warfare agent.

In some embodiments, where the composition of the invention is a nucleic acid encoding a prophylactic or therapeutic agent, the nucleic acid can be administered in vivo to promote expression of their encoded proteins (e.g., CG53135 proteins), by constructing the nucleic acid as part of an appropriate nucleic acid expression vector and administering it so that it becomes intracellular, e.g., by use of a retroviral vector, or by direct injection, or by use of microparticle bombardment (e.g., a gene gun), or coating with lipids or cell-surface receptors or transfecting agents, or by administering it in linkage to a homeobox-like peptide which is known to enter the nucleus, etc. Alternatively, a nucleic acid of the invention can be introduced intracellularly and incorporated within host cell DNA for expression, by homologous recombination. The instant invention encompasses bulk drug compositions useful in the manufacture of pharmaceutical compositions that can be used in the preparation of unit dosage forms. In a preferred embodiment, a composition of the invention is a pharmaceutical composition. Such compositions comprise a prophylactically or therapeutically effective amount of CG53135, and a pharmaceutically acceptable carrier. Preferably, the pharmaceutical compositions are formulated to be suitable for the route of administration to a subject.

In one embodiment, the term “pharmaceutically acceptable” means approved by a regulatory agency of the Federal or a state government or listed in the U.S. Pharmacopeia or other generally regarded as safe for use in humans (GRAS). The term “carrier” refers to a diluent, adjuvant, bulking agent (e.g., arginine in various salt forms, sulfobutyl ether Beta-cyclodextrin sodium, or sucrose), excipient, or vehicle with which CG53135 is administered. Such pharmaceutical carriers can be sterile liquids, such as water and oils (e.g., oils of petroleum, animal, vegetable or synthetic origins, such as peanut oil, soybean oil, mineral oil, sesame oil and the like), or solid carriers, such as one or more substances which may also act as diluents, flavoring agents, solubilizers, lubricants, suspending agents, or encapsulating material. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include, but are not limited to, starch or its synthetically modified derivatives such as hydroxyethyl starch, stearate salts, glycerol, glucose, lactose, sucrose, trehalose, gelatin, sulfobutyl ether Beta-cyclodextrin sodium, sodium chloride, glycerol, propylene, glycol, water, ethanol, or a combination thereof. The composition, if desired, can also contain minor amounts of wetting or emulsifying agents, or pH buffering agents.

The compositions comprising CG53135 may be formulated into any of many possible dosage forms such as, but not limited to, liquid, suspension, microemulsion, microcapsules, tablets, capsules, gel capsules, soft gels, pills, powders, enemas, sustained-release formulations and the like. The compositions comprising CG53135 may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. The composition can also be formulated as a suppository, with traditional binders and carriers such as triglycerides. Oral formulation can include standard carriers, such as pharmaceutical grades of mannitol, lactose, starch or its synthetically modified derivatives such as hydroxyethyl starch, stearate salts, sodium saccharine, cellulose, magnesium carbonate, etc.

A pharmaceutical composition comprising CG53135 is formulated to be compatible with its intended route of administration. In a specific embodiment, the composition is formulated in accordance with routine procedures as a pharmaceutical composition adapted for intravenous, subcutaneous, intramuscular, oral, intranasal, intratumoral or topical administration to human beings. Typically, compositions for intravenous administration are solutions in sterile isotonic or hypertonic aqueous buffer. Where necessary, the composition may also include a solubilizing agent and a local anesthetic such as benzyl alcohol or lidocaine to ease pain at the site of the injection.

If a composition comprising CG53135 is to be administered topically, the composition can be formulated in the form of transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the compositions of the invention are in admixture with a topical delivery agent, such as but not limited to, lipids, liposomes, fatty acids, fatty acid esters, steroids, chelating agents and surfactants. The compositions comprising CG53135 may be encapsulated within liposomes or may form complexes thereto, in particular to cationic liposomes. Alternatively, the compositions comprising CG53135 may be complexed to lipids, in particular to cationic lipids. For non-sprayable topical dosage forms, viscous to semi-solid or solid forms comprising a carrier or one or more excipients compatible with topical application and having a dynamic viscosity preferably greater than water are typically employed. Other suitable topical dosage forms include sprayable aerosol preparations wherein the active ingredient, preferably in combination with a solid or liquid inert carrier, is packaged in a mixture with a pressurized volatile (e.g., a gaseous propellant, such as Freon or hydrofluorocarbons) or in a squeeze bottle. Moisturizers or humectants can also be added to pharmaceutical compositions and dosage forms if desired. Examples of such additional ingredients are well-known in the art.

A composition comprising CG53135 can be formulated in an aerosol form, spray, mist or in the form of drops or powder if intranasal administration is preferred. In particular, a composition comprising CG53135 can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, other hydrofluorocarbons, carbon dioxide or other suitable gas). In the case of a pressurized aerosol the dosage unit may be determined by providing a valve to deliver a metered amount. Microcapsules (composed of, e.g., polymerized surface) for use in an inhaler or insufflator may be formulated containing a powder mix of the compound and a suitable powder base such as dissacharides or starch.

One or more CG53135 proteins may also be formulated into a microcapsule with one or more polymers (e.g., hydroxyethyl starch) form the surface of the microcapsule. Such formulations have benefits such as slow-release.

A composition comprising CG53135 can be formulated in the form of powders, granules, microparticulates, nanoparticulates, suspensions or solutions in water or non-aqueous media, capsules, gel capsules, sachets, tablets or minitablets if oral administration is preferred. Thickeners, flavoring agents, diluents, emulsifiers, dispersing aids or binders may be desirable. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

In one embodiment, the compositions of the invention are orally administered in conjunction with one or more penetration enhancers, e.g., alcohols, surfactants and chelators. Preferred surfactants include, but are not limited to, fatty acids and esters or salts thereof, bile acids and salts thereof. In some embodiments, combinations of penetration enhancers are used, e.g., alcohols, fatty acids/salts in combination with bile acids/salts. In a specific embodiment, sodium salt of lauric acid, capric acid is used in combination with UDCA. Further penetration enhancers include, but are not limited to, polyoxyethylene-9-lauryl ether, polyoxyethylene-20-cetyl ether. Compositions of the invention may be delivered orally in granular form including, but is not limited to, sprayed dried particles, or complexed to form micro or nanoparticles. Complexing agents that can be used for complexing with the compositions of the invention include, but are not limited to, poly-amino acids, polyimines, polyacrylates, polyalkylacrylates, polyoxethanes, polyalkylcyanoacrylates, cationized gelatins, albumins, acrylates, polyethyleneglycols (PEG), DEAE-derivatized polyimines, pollulans, celluloses, and starches. Particularly preferred complexing agents include, but are not limited to, chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

A composition comprising CG53135 can be delivered to a subject by pulmonary administration, e.g., by use of an inhaler or nebulizer, of a composition formulated with an aerosolizing agent.

In a preferred embodiment, a composition comprising CG53135 is formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as benzyl alcohol or lidocaine to ease pain at the site of the injection. Generally, the ingredients are supplied either separately or mixed together in unit dosage form, for example, as a dry lyophilized powder or water free concentrate in a sealed container, such as a vial, ampoule or sachette, indicating the quantity of active agent. Where the composition is to be administered by infusion, it can be dispensed with an infusion container containing sterile pharmaceutical grade water or saline. Where the composition is administered by injection, an ampoule or vial of sterile water for injection or saline can be provided so that the ingredients may be mixed prior to administration.

A composition comprising CG53135 can be formulated as neutral or salt forms. Pharmaceutically acceptable salts include, but are not limited to, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In addition to the formulations described previously, a composition comprising CG53135 may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In one embodiment, the ingredients of the compositions used in accordance to the methods of the invention are derived from a subject that is the same species origin or species reactivity as recipient of such compositions.

In some embodiments, a formulation used in accordance to the methods of the invention comprises 0.02 M-0.2 M acetate, 0.5-5% glycerol, 0.2-0.5 M arginine-HCl, and one ore more CG53135 proteins, preferably 0.5-5 mg/ml (UV). In one embodiment, a formulation used in accordance to the methods of the invention comprises 0.04M sodium acetate, 3% glycerol (volume/volume), 0.2 M arginine-HCl at pH 5.3, and one or more isolated CG53135 proteins, preferably 0.8 mg/ml (UV). In some embodiments, a formulation used in accordance to the methods of the invention comprises 0.01-1 M of a stabilizer, such as arginine in various salt forms, sulfobutyl ether Beta-cyclodextrin sodium, or sucrose, 0.01-0.1 M sodium phosphate monobasic (NaH₂PO₄.H₂O), 0.01%-0.1% weight/volume (“w/v”) polysorbate 80 or polysorbate 20, and one or more CG53135 proteins, preferably 0.005-50 mg/ml (UV). In one embodiment, a formulation used in accordance to the methods of the invention comprises 30 mM sodium citrate, pH 6.1, 2 mM EDTA, 200 mM sorbitol, 50 mM KCl, 20% glycerol, and one or more isolated CG53135 proteins.

The invention also provides kits for carrying out the therapeutic regimens of the invention. Such kits comprise in one or more containers prophylactically or therapeutically effective amounts of the composition of the invention (e.g., a composition comprising one or more CG53135 proteins) in pharmaceutically acceptable form. The composition in a vial of a kit of the invention may be in the form of a pharmaceutically acceptable solution, e.g., in combination with sterile saline, dextrose solution, or buffered solution, or other pharmaceutically acceptable sterile fluid. Alternatively, the composition may be lyophilized or desiccated; in this instance, the kit optionally further comprises in a container a pharmaceutically acceptable solution (e.g., saline, dextrose solution, etc.), preferably sterile, to reconstitute the composition to form a solution for injection purposes.

In another embodiment, a kit of the invention further comprises a needle or syringe, preferably packaged in sterile form, for injecting the formulation, and/or a packaged alcohol pad. Instructions are optionally included for administration of the formulations of the invention by a clinician or by the patient.

In some embodiments, the present invention provides kits comprising a plurality of containers each comprising a pharmaceutical formulation or composition comprising a dose of the composition of the invention (e.g., a composition comprising one or more CG53135 proteins) sufficient for a single administration.

As with any pharmaceutical product, the packaging material and container are designed to protect the stability of the product during storage and shipment. In one embodiment, compositions of the invention are stored in containers with biocompatible detergents, including but not limited to, lecithin, taurocholic acid, and cholesterol; or with other proteins, including but not limited to, gamma globulins and serum albumins. Further, the products of the invention include instructions for use or other informational material that advise the physician, technician, or patient on how to appropriately prevent or treat the disease or disorder in question.

### 6. EXAMPLES

The present invention is further illustrated by the following non-limiting examples.

### 6.1. Example 1

### Protein Expression and Purification

Recombinant human CG53135 was purified from Escherichia coli BLR (DE3) cells (Novagen, Madison, Wis.) as follows: the DE3 cells were transformed with full-length, codon-optimized CG53135-05 cloned in a pET24a vector (Novagen), and a manufacturing master cell bank (MMCB) of these cells was produced. Cell paste containing CG53135-05 produced by fermentation of cells originating from the MMCB was lysed with high-pressure homogenization in lysis buffer and clarified by centrifugation. CG53135-05 was purified from clarified cell lysate by two cycles of ion exchange chromatography and ammonium sulfate precipitation. The final protein fraction was dialyzed against the formulation buffer (30 mM citrate, pH 6.0, 2 mM ethylenediaminetetraacetic acid (EDTA), 200 mM sorbitol, 50 mM KCl, and 20% glycerol). Vehicle (without CG53135 protein) contains 30 mM sodium citrate, pH 6.1, 2 mM EDTA, 200 mM sorbitol, 50 mM KCl, 20% glycerol.

Other types of formulations and purification methods can be found in Section 6.18, infra.

Analysis of the final purified protein product using Liquid Chromatography, Mass spectrometry and N-terminal sequencing indicate that the final purified protein product includes some truncated form of FGF-20 (e.g., CG53135-13 (SEQ ID NO:24), CG53135-15 (SEQ ID NO:28), CG53135-16 (SEQ ID NO:30), and CG53135-17 (SEQ ID NO:32)) in addition to the full length FGF-20, and a protein consisting of amino acid 3-211 (CG53135-13, SEQ ID NO:24) of FGF-20 constitutes the majority of the final purified protein product.

All the variants/fragments in the final purified product have high activity in the proliferation assays. Thus these variants/fragments are expected to have same utility as that of FGF-20. For the purpose of convenience, the term “CG53135-05 E. coli purified product” is used herein to refer to a purified protein product from E. coli expressing a CG53135-05 construct. For example, a CG53135-05 E coli purified product may contain a mixture of the full length CG53135-05 protein (SEQ ID NO:2), CG53135-13 (SEQ ID NO:24), CG53135-15 (SEQ ID NO:28), CG53135-16 (SEQ ID NO:30), and CG53135-17 (SEQ ID NO:32), with the majority of the content being CG53135-13 (SEQ ID NO:24). A CG53135 E. coli purified product may also contain one or more carbamylated CG53135 proteins.

RP-PLC Assay: Peak Identification

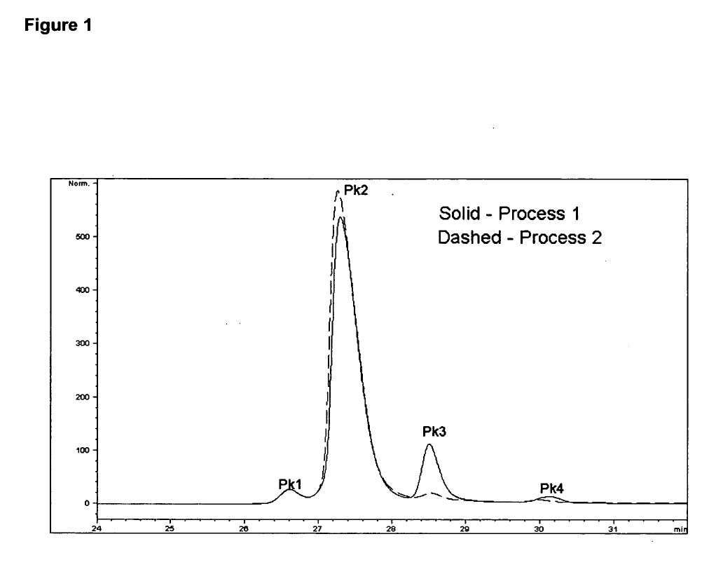

Purified drug substance (by both Process 1 and Process 2, respectively, see Section 6.14, infra) was further analyzed by reversed-phase high-performance liquid chromatography (RP-HPLC) with both UV and electrospray mass spectrometric detection. Purified protein from either Process 1 or Process 2 was loaded onto a Protein C4 column (Vydac, 5 μm, 150 mm×4.6 mm) using a standard HPLC system in a mobile phase containing water, acetonitrile and trifluoroacetic acid. The elution gradient for this method was modified to resolve four distinct chromatographic peaks eluting at 26.6, 27.3, 28.5 and 30.0 min respectively (FIG. 1). These peaks were characterized by electrospray mass spectrometry. As can be observed from the chromatograms, the four equipotent isoforms are present in the purified final product from Process 1 and 2. However, the proportion of these peaks (1, 3 and 4) is much lower in the final product purified by Process 2 with the predominant form being Peak 2.

The identities of each peak from the RP-HPLC separation are indicated in Table 2.

TABLE 2
Identity of peaks from the RP-HPLC separation of CG53135-05 E. coli
purified product based upon accurate molecular weight determination.

| | | Molecular | | | Predicted |
|---|---|---|---|---|---|
| | Retention | Weight | | | Molecular |
| Peak # | Time (min) | Observed | Assignment (residue #) | ID Number | Weight |
| 1 | 26.6 | 21329.2 | 24-211 | CG53135-17 | 21329.2 |
| 1 | 26.6 | 22185.1 | 15-211 | CG53135-16 | 22185.1 |
| 1 | 26.6 | 22412.4 | 12-211 | CG53135-15 | 22412.4 |
| 2 | 27.3 | 23296.5 | 3-211 | CG53135-13 | 23296.4 |
| 3 | 28.5 | 23498.9 | 1-211 | CG53135-05 | 23498.7 |
| 4 | 30.0 | 23339.3 | 3-211 | CG53135-13 | 23339.4 |
| | | | (carbamylated) | (carbamylated) | |
| 4 | 30.0 | 23539.7 | 1-211 | CG53135-05 | 23539.7 |
| | | | (carbamylated) | (carbamylated) | |

Edman Sequencing and Total Amino Acid Analysis

The experimental N-terminal amino acid sequence of the Process 1 reference standard, DEV10, and the Process 2 interim reference standard were determined qualitatively. The reference standards were resolved by SDS-PAGE and electrophoretically transferred to a polyvinylidenefluoride membrane; the Coomassie-stained ˜23 kDa major band corresponding to each reference standard was excised from the membrane and analyzed by an automated Edman sequencer (Procise, Applied Biosystems, Foster City, Calif.). A comparison of the two major sequences is shown in Table 3 below. The predominant sequence for each reference standard was identical and corresponded to residues 3-20 in the theoretical N-terminal sequence of CG53135-05.

TABLE 3
Edman sequencing data for the first 20 amino
acids of CG53135-05 E. coli purified product
for Process 1 and 2.

| | |
|---|---|
| Theoretical | |
| Residue | Amino Acid Residue |

| Position | Process 1 | Process 2 |
|---|---|---|
| 3 | Pro | Pro |
| 4 | Leu | Leu |
| 5 | Ala | Ala |
| 6 | Glu | Glu |
| 7 | Val | Val |
| 8 | Gly | Gly |
| 9 | Gly | Gly |
| 10 | Phe | Phe |
| 11 | Leu | Leu |
| 12 | Gly | Gly |
| 13 | Gly | Gly |
| 14 | Leu | Leu |
| 15 | Glu | Glu |
| 16 | Gly | Gly |
| 17 | Leu | Leu |
| 18 | Gly | Gly |
| 19 | Gln | Gln |
| 20 | Gln | Gln |

The experimental amino acid composition of the DEV10 reference standard and the PX3536G001-H reference standard were determined in parallel. Quadruplicate samples of each reference standard were hydrolyzed for 16 hours at 115° C. in 100 μL of 6 N HCl, 0.2% phenol containing 2 nmol norleucine as an internal standard. Samples were dried in a Speed Vac Concentrator and dissolved in 100 μL sample buffer containing 2 nmol homoserine as an internal standard. The amino acids in each sample were separated on a Beckman Model 7300 amino acid analyzer. The amino acid composition of both reference standards showed no significant differences as shown in Table 4 below. Note that Cys and trp are destroyed during acid hydrolysis of the protein. Asn and gln are converted to asp and glu, respectively, during acid hydrolysis and thus their respective totals are reported as asx and glx. Met and his were both unresolved in this procedure.

TABLE 4
Quantitive amino acid analysis for CG53135-05 E. coli
purified product from Process 1 and Process 2

| | |
|---|---|
| Amino | |
| Acid | Mole Percent |

| Residue | DEV10 | PX3536G001-H |
|---|---|---|
| asx | 7.1 | 7.0 |
| thr | 4.0 | 4.0 |
| ser | 6.3 | 6.1 |
| glx | 12.2 | 12.2 |
| pro | 6.0 | 6.0 |
| gly | 14.4 | 14.3 |
| ala | 5.8 | 5.6 |
| val | 5.3 | 5.3 |
| ile | 3.5 | 3.5 |
| leu | 13.6 | 13.6 |
| tyr | 4.6 | 4.6 |
| phe | 5.2 | 5.2 |
| lys | 3.7 | 3.7 |
| arg | 8.5 | 9.1 |

Tryptic Mapping by RP-HPLC

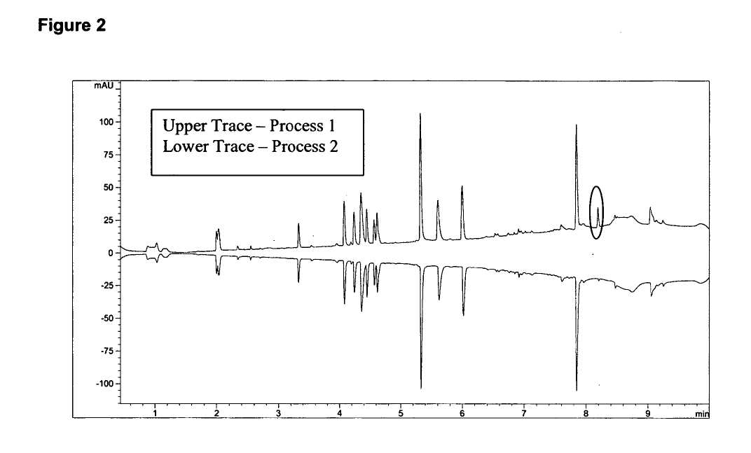

Purified drug substance from Process 1 and 2 was reduced and alklated with iodoacetic acid and then digested with sequencing grade trypsin. The tryptic peptides were separated by reversed-phase high-performance liquid chromatography (RP-HPLC) using both UV and electrospray mass spectrometric detection. The tryptic digest from either Process 1 or Process 2 was loaded onto an ODS-1 nonporous silica column (Micra, 1.5 μm; 53×4.6 mm) using a standard HPLC system in a mobile phase containing water, acetonitrile and trifluoroacetic acid. The eluting peptides were detected by UV absorption at 214 nm (FIG. 2) and by positive-ion electrospray mass spectrometry. The major difference between the two chromatograms for Process 1 and Process 2 is the reduction in peak area of a peak obvious in the Process 1 trace (peak at 8.2 min; FIG. 2). This peak corresponds to the T1 peptide, residues 1-40. This observation is expected since the source of this peptide if from the intact CG53135-05, which is in greater abundance in the Process 1 material (peak 3, FIG. 1).

Bioassay

The biological activity of CG53135-05 related species collected from the 4 peaks identified by LC and MS was measured by treatment of serum-starved cultured NIH 3T3 murine embryonic fibroblast cells with various doses of the isolated CG53135-05 related species and measurement of incorporation of bromodeoxyuridine (BrdU) during DNA synthesis. For this assay, cells were cultured in Dulbecco's modified Eagle's medium supplemented with 10% fetal bovine serum. Cells were grown in 96-well plates to confluence at 37° C. in 10% CO₂/air and then starved in Dulbecco's modified Eagle's medium for 24-72 hours. CG53135-05-related species were added and incubated for 18 hours at 37° C. in 10% CO₂/air. BrdU (10 mM final concentration) was added and incubated with the cells for 2 hours at 37° C. in 10% CO₂/air. Incorporation of BrdU was measured by enzyme-linked immunosorbent assay according to the manufacturer's specifications (Roche Molecular Biochemicals, Indianapolis, Ind.).

Peak 4 was hot included in this assay since insufficient material was collected (Peak 4 is less than 3% of the total peak area for CG53135-05). CG53135-05 and material collected from all 3 remaining fractions (i.e., Peak 1, 2, and 3) induced DNA synthesis in NIH 3T3 mouse fibroblasts in a dose-dependent manner (Table 5). The P1200 was defined as the concentration of protein that resulted in incorporation of BrdU at 2 times the background. CG53135-05 and CG53135-05 related species recovered from all 3 measurable peaks demonstrated similar biological activity with a PI₂₀₀ of 0.7-11 ng/mL (Table 5).

TABLE 5
Biological Activity of CG53135-05 (DEV10):
Induction of DNA Synthesis

| | CG53135-05 | | | |
|---|---|---|---|---|
| | (DEV 10 | Peak 1 | Peak 2 | Peak 3 |
| PI₂₀₀ (ng/mL) | 1.0 | 0.7 | 11 | 8.6 |

### 6.2. Example 2

### Prophylactic Protective Effects of CG53135 From Radiation Exposure

6.2.1. Effect of CG53135-05 on Survival After Single Exposure to Acute Radiation (Study N-272)

This study was performed to investigate the effect of the CG53135-05 E. coli purified product administered with diferent schedules as a radioprotectant in mice after lethal total body ionizing radiation. The protein concentration in this example was measure by Bradford assay.

Male C3H/He mice (total number “n”=60) with an average weight of 22.1 gram at study initiation were used for treatment groups. Animals were fed with a standard commercial mouse diet. Food and water were provided ad libitum.

Study Design

Irradiation of the animals was performed at the Brigham and Women's Hospital in Boston, Mass. The studies were conducted at The Massachusetts College of Pharmacy, Boston, Mass. Animals were randomly divided into 4 groups with a control group of 15 mice and 3 test groups of 15 mice each (Table 6). Mice were exposed to a single dose of 600 cGy of total body ionizing radiation. CG53135-05 (12 mg/kg) was administered intraperitoneally (IP) daily on Days 2-3 after radiation, Days 4-7 after radiation, or Days 2 and 1 (−2 and −1) before radiation, respectively. Mortality (animal number, day of death) was recorded in each group.

TABLE 6
Study Design

| | Number of | | | |
|---|---|---|---|---|
| Group | Male | Induction | | Treatment |
| Number | Animals | Day 0 | Treatment | Schedule |
| 1 | 15 | 600 cGy | Vehicle | None |
| 2 | 15 | 600 cGy | CG53135-05 E. coli | Daily, Days 2-3 |
| | | | purified product | |
| | | | 12 mg/kg (Bradford) | |
| 3 | 15 | 600 cGy | CG53135-05 E. coli | Daily, Days 4-7 |
| | | | purified product | |
| | | | 12 mg/kg (Bradford) | |
| 4 | 15 | 600 cGy | CG53135-05 E. coli | Daily, Days −2 |
| | | | purified product | and −1 |
| | | | 12 mg/kg (Bradford) | |

All animals were given CG53135-05 or vehicle (0.1 mL/10 g body weight) once daily.

Statistics

Survival of treatment groups was compared using a Kaplan-Meier log-rank analysis. Weight change was analyzed by determining the area under the curve (AUC) using a trapezoidal rule transformation. The area under the curve was calculated using the equation,
i={yi(x+1−xi)+(1/2)(yi+1−yi)(x+1−xi)}
where x=time (d), y=percent weight change, and i=time point, from which it follows that xi is the value of x at time point i, yi is the value of y at that time point, and that xi+1 and yi+1 are the values of x and y at the next time point. Both the t-test and Mann-Whitney Rank Sum analysis were used to compare the AUC for weight change between different treatment groups.
Results

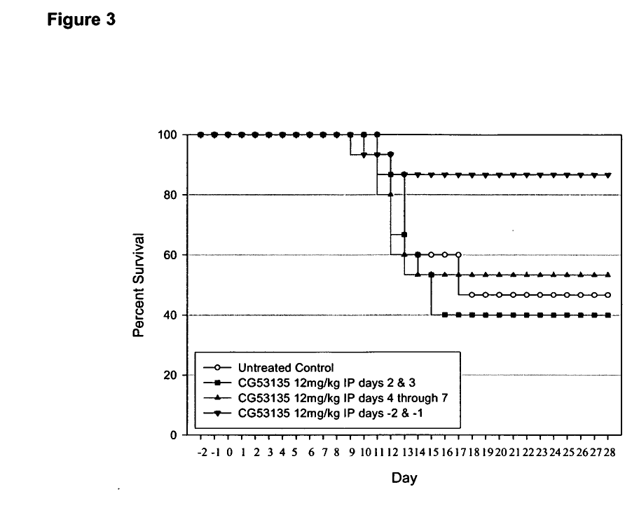

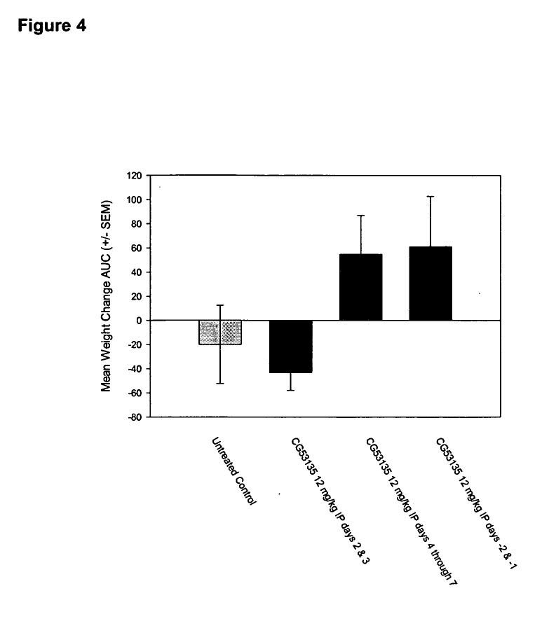

Treatment of mice with the CG53135-05 E. coli purified product (12 mg/kg, Bradford) on Days −2 and −1 before radiation significantly increased survival compared with untreated controls (P=0.04), whereas treatment with the CG53135-05 E. coli purified product on Days 2-3 or Days 4-7 after radiation did not have a significant effect on survival (P>0.05) (FIG. 3). Furthermore, the prophylactic dose of CG53135-05 (on Days −2 and −1) also prevented weight loss post-radiation (FIG. 4). Treatment with CG53135-05 on Days −2 and −1 (13.9%) and Days 4-7 (14.8%) also resulted in increased overall mean percent weight gains compared with untreated controls (9.7%), although statistical analysis of the area under the curve (AUC) for weight change by one-way ANOVA indicated no significant differences in the groups (P=0.051) (FIG. 4). The results of this study indicate that treatment of mice with CG53135-05 on Days −1 and −2 prior to total body irradiation is effective in radioprotection and also supports the radioprotective effect on gastrointestinal or hematopoietic tissues from the damaging effects of total body irradiation.

6.2.2. Prophylactic Effect of the CG53135-05 E. coli Purified Product on Mice After Exposure to Acute Ionizing Radiation (Study N-308)

This study was performed to investigate the effect of the CG53135-05 E. coli purified product administered prophylactically to mice that later were exposed to various doses of total body ionizing radiation. The procedures that were followed were the same as detailed in Section 6.2.1. Protein concentration was measured by Bradford assay. Mice were exposed to ionizing radiation without anesthesia at a dose range of 484 to 641 cGy on day 0. Animals were dosed with PBS (control) or the CG53135-05 E. coli purified product (12 mg/kg, Bradford, daily IP) on day −1, or days −2 and −1 before radiation. The schedule is represented in Table 7. The endpoints for the study were survival and weight changes. Survival was followed for 30 days post-irradiation.

TABLE 7
Study Design

| Group | Number of | | | Treatment |
|---|---|---|---|---|
| Number | Animals | Induction | Treatment | Schedule* |
| 1 | 16 males | 484 cGy Day 0 | PBS | Day −2, −1 |
| 2 | 16 males | 534 cGy Day 0 | PBS | Day −2, −1 |
| 3 | 16 males | 570 cGy Day 0 | PBS | Day −2, −1 |
| 4 | 16 males | 606 cGy Day 0 | PBS | Day −2, −1 |
| 5 | 16 males | 641 cGy Day 0 | PBS | Day −2, −1 |
| 6 | 16 males | 484 cGy Day 0 | CG53135-05 E. coli | Day −1 |
| | | | purified product, 12 | |
| | | | mg/kg, Bradford, IP | |
| 7 | 16 males | 534 cGy Day 0 | CG53135-05 E. coli | Day −1 |
| | | | purified product, 12 | |
| | | | mg/kg, Bradford, IP | |
| 8 | 16 males | 570 cGy Day 0 | CG53135-05 E. coli | Day −1 |
| | | | purified product, 12 | |
| | | | mg/kg, Bradford, IP | |
| 9 | 16 males | 606 cGy Day 0 | CG53135-05 E. coli | Day −1 |
| | | | purified product, 12 | |
| | | | mg/kg, Bradford, IP | |
| 10 | 16 males | 641 cGy Day 0 | CG53135-05 E. coli | Day −1 |
| | | | purified product, 12 | |
| | | | mg/kg, Bradford, IP | |
| 11 | 16 males | 484 cGy Day 0 | CG53135-05 E. coli | Day −2, −1 |
| | | | purified product, 12 | |
| | | | mg/kg, Bradford, IP | |
| 12 | 16 males | 534 cGy Day 0 | CG53135-05 E. coli | Day −2, −1 |
| | | | purified product, 12 | |
| | | | mg/kg, Bradford, IP | |
| 13 | 16 males | 570 cGy Day 0 | CG53135-05 E. coli | Day −2, −1 |
| | | | purified product, 12 | |
| | | | mg/kg, Bradford, IP | |
| 14 | 16 males | 606 cGy Day 0 | CG53135-05 E. coli | Day −2, −1 |
| | | | purified product, 12 | |
| | | | mg/kg, Bradford, IP | |
| 15 | 16 males | 641 cGy Day 0 | CG53135-05 E. coli | Day −2, −1 |
| | | | purified product, 12 | |
| | | | mg/kg, Bradford, IP | |

Results:

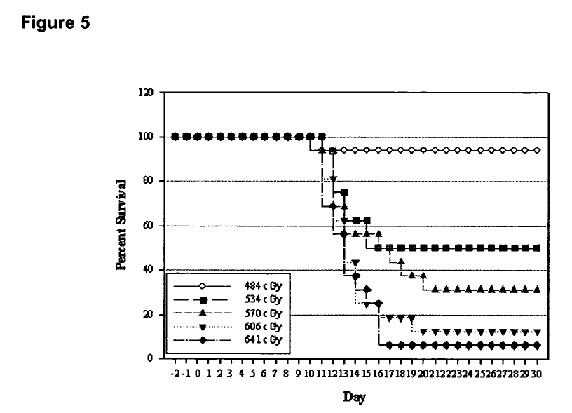

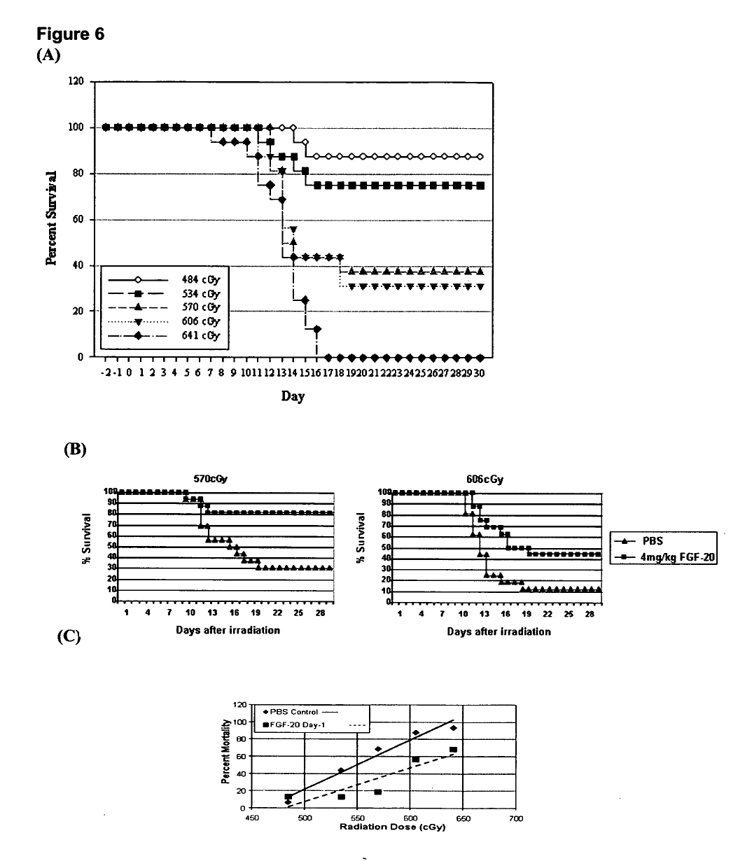

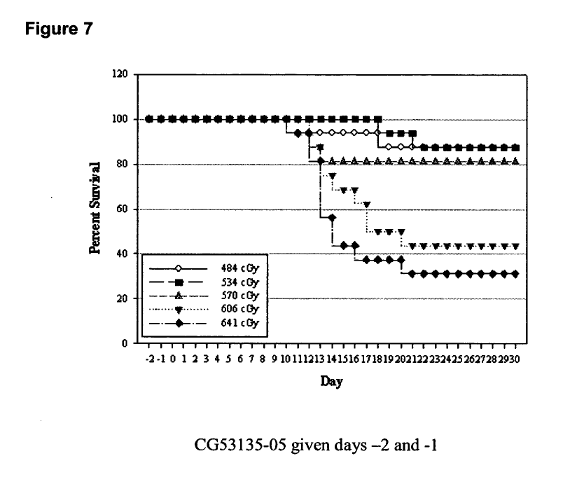

Survival decreased as radiation dose increased in all treatment groups. In animals receiving PBS, 30-day survival at the lowest dose of radiation (484 cGy) was 93.75%, and decreased to 50.0% at 534 cGy, 31.25% at 570 cGy, 12.5% at 606 cGy, and 6.25% at 641 cGy (FIG. 5). In animals receiving CG53135, 12 mg/kg IP on Day −1 only, the 30-day survival at the lowest dose of radiation (484 cGy) was 87.5%, compared to 87.5% at 534 cGy, 81.25% at 570 cGy, 43.75% at 606 cGy, and 31.25% at 641 cGy (FIG. 6). In animals receiving GC53135, 12 mg/kg IP on Days −2 and −1, the 30-day survival at the lowest dose of radiation (484 cGy) was 87.5%, compared to 75.0% at 534 cGy, 37.5% at 570 cGy, 31.25% at 606 cGy, and zero at 641 cGy (FIG. 7).

A multiple comparison test demonstrated a 4.8-fold increase in the odds of survival in animals treated on day −1 versus control animals (p=0.00016). LD_{50/30} values were calculated using a probit plot of survivorship with 95% confidence intervals calculated by bootstrapping. However, the odds of survival in animals treated on day −2 and −1 versus control animals were not significant (p=0.4162). The results are indicative of the therapeutic effect of prophylactic administration of CG53135-05 in radioprotection. Further, one day treatment before radiation (day −1) also protected animals from weight loss in all but the highest radiation level (641 cGy). In this particular system, single dose (on day −1) was significantly more effective than the two-dose regimen (on days −2 and −1, respectively) especially in higher radiation levels.

In addition to the above results, the invention could be extended to additional dose regimens of the CG53135-05 E. coli purified product, such as prophylactically and/or therapeutically administer the CG53135-05 E. coli purified product prior and/or after the radiation exposure, which could be tested in the same animal model following the same procedures as described herein, in order to define the range of therapeutic efficacy of this compound. The dose regimen for therapeutic treatment may include, but is not limited to, +1, +1, and +2 days after radiation exposure. For example, in another experiment, mice were dosed IP with 4 mg/kg (UV) CG53135-05 E. coli purified product 24 hours prior to whole-body irradiation at the indicated doses. The survival of the mice was then followed for 30 days. FIG. 6B shows the Kaplan-Meier plots for survival at 570 cGy and 606 cGy with statistically significant differences between the group treated with the CG53135-05 E. coli purified product and the control group, i.e., p=0.008 and p=0.015, respectively. FIG. 6C shows probit analysis for survival over the range of doses. The LD_{50/30} for control and animals treated with the CG53135-05 E. coli purified product is 552.4 cGy and 607.4 cGy, respectively, with a dose modification factor (DMF) of 1.10.

### 6.3. Example 3

### Modulation of Intestinal Crypt Cell Proliferation and Apoptosis by CG53135-05 Administration to Mice (Study N-342)

This study evaluated the effect of CG53135 on small intestinal crypt cell turnover in order to discriminate stem cell versus daughter cell effects, and to draw insights regarding the mode of action of CG53135 in syndromes associated with gastrointestinal stem cell damage (e.g., mucositis). Furthermore, the effect of CG53135 on stem cell radiosensitivity was also assessed. Protein concentrations in this example were measured by Bradford assay.

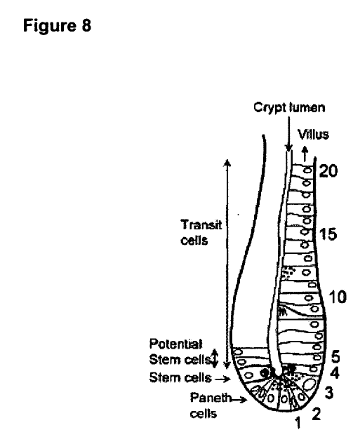

A “crypt” is a hierarchical structure with the stem cells towards the crypt base. As cells become more mature, they move progressively from the bottom of the crypt towards the top of the crypt. Therefore, changes that may be affecting stem cells versus their transit amplifying daughter cells can be detected by looking at changes in event frequency at each cell position. The cell positions are marked in FIG. 8. Thus, the effects of CG53135 on the crypt microarchitecture were analyzed in the context of crypt cellularity.

Experimental Design

Animals were sacrificed at various times after a single 12 mg/kg (Bradford, IP) dose of the CG53135-05 E. coli purified product. Just prior to sacrifice the mice were labeled with a single injection of bromodeoxyuridine to label S-phase cells and determine the effect of the drug on crypt cell proliferation/apoptosis. Two further groups of mice were used to assess effects on stem cell radiosensitivity. One group was treated with the CG53135-05 E. coli purified product (12 mg/kg, Bradford single injection, IP) and another group was injected with a placebo control. Twenty-four hours post injection, animals were irradiated with 1 Gy X-ray (specifically to induce stem cell apoptosis) followed by routine in vivo BrdU labeling. Animals were sacrificed 4.5 hours later (at time of peak apoptosis).

Mice were weighed and then dosed with the CG53135-05 E. coli purified product (12 mg/kg, Bradford, single injection, IP). Groups of 6 animals were sacrificed 0, 3, 6, 9, 12, 24, 48 hours post injection with CG53135-05. All received a single injection of bromodeoxyuridine 40 minutes prior to sacrifice (see Table 8).

An additional two groups of 6 mice were used to assess the effects of CG53135-05 on stem cell radiosensitivity (groups 8 and 9, see Table 8). One group was treated with CG53135-05 (12 mg/kg Bradford, single injection, ip) and one group was injected with a placebo control. 24 hours post injection, animals were irradiated with 1 Gy X-ray and sacrificed 4.5 hours later.

TABLE 8
Study Design

| Group | Number of | | Treatment |
|---|---|---|---|
| Number | Animals | Treatment | Schedule* |
| 1 | 6 males | CG53135-05 E. coli | Injected and euthanize 3 hr later |
| | | purified product, | 40 mg/kg BrdU 40 min prior to sacrifice |
| | | 12 mg/kg, IP | |
| 2 | 6 males | CG53135-05 E. coli | Injected and euthanize 6 hr later |
| | | purified product, | 40 mg/kg BrdU 40 min prior to sacrifice |
| | | 12 mg/kg, IP | |
| 3 | 6 males | CG53135-05 E. coli | Injected and euthanize 9 hr later |
| | | purified product, | 40 mg/kg BrdU 40 min prior to sacrifice |
| | | 12 mg/kg, IP | |
| 4 | 6 males | CG53135-05 E. coli | Injected and euthanize 12 hr later |
| | | purified product, | 40 mg/kg BrdU 40 min prior to sacrifice |
| | | 12 mg/kg, IP | |
| 5 | 6 males | CG53135-05 E. coli | Injected and euthanize 24 hr later |
| | | purified product, | 40 mg/kg BrdU 40 min prior to sacrifice |
| | | 12 mg/kg, IP | |
| 6 | 6 males | CG53135-05 E. coli | Injected and euthanize 48 hr later |
| | | purified product, | 40 mg/kg BrdU 40 min prior to sacrifice |
| | | 12 mg/kg, IP | |
| 7 | 6 males | Untreated | 40 mg/kg BrdU 40 min prior to sacrifice |
| 8 | 6 males | CG53135-05 E. coli | Dose 24 hr prior to irradiation |
| | | purified product, | Euthanize 4.5 hr post irradiation |
| | | 12 mg/kg, IP 1Gy X ray | 40 mg/kg BrdU 40 min prior to sacrifice |
| 9 | 6 males | PBS, IP 1Gy X-ray | Dose 24 hr prior to irradiation |
| | | | Euthanize 4.5 hr post irradiation |
| | | | 40 mg/kg BrdU 40 min prior to sacrifice |

Intestinal Crypt Cell Proliferation and Apoptosis Modulation: Procedure

All S-phase dividing cells incorporate the injected Bromodeoxyuridine (BrdU) and hence were marked as cycling cells. Animals that were irradiated were placed, unanaesthetised, in a perspex jig and subjected to whole body radiation of 1 Gy X-ray at a dose rate of 0.7 Gy/min. This low level of radiation induced apoptosis in the small intestinal stem cell population, but not in the more mature cells.

The small intestine was removed, fixed in Carnoy's fixative, and processed for histological analysis (paraffin embedded). One set of 3 mm sections were immunolabeled for BrdU and one set of sections were stained with H&E. Longitudinal sections of small intestinal crypts were analyzed for the presence either BrdU or apoptotic/mitotic nuclei. Fifty half crypts were scored per animal.

Groups 1-7 (Group A in the results) were tested to determine the effect of the CG53135-05 E. coli purified product over a 48 hour period. Groups 8-9 (Group B in the results) were tested to determine whether the CG53135-05 E. coli purified product changes the number of apoptotic cells generated after low dose irradiation, i.e., whether the CG53135-05 E. coli purified product influences the radiosensitive stem cell population.

The results generated show a frequency distribution for the crypts in each group of animals that were further analyzed for statistical differences. Tissue samples were harvested at 3, 6, 9, 12, 24, and 48 hours after treatment with the CG53135-05 E. coli purified product. Apoptosis, mitotic index, and proliferation were the end points for this study.

Results:

Group A

In groups 1-7 (Table 8), the CG53135-05 E. coli purified product had no significant effect on spontaneous apoptosis. Similar results were obtained with the mitotic index (Table 9). However, results of BrdU uptake as in Table 9, revealed the following:
a) At 3 hour, there was extension/increase of proliferative region (cell positions 12-22).
b) By 9 hours, large proliferative effects were noted in many cell positions.
c) By 12 hours, only cell positions 4-8 showed increase in uptake (stem cells).
d) By 24 hours, there was a significant inhibition of proliferation.

e) By 48 hours, the uptake was comparable to control levels.

TABLE 9
Summary of significant cell positions in the crypt after
assessment of apoptosis, mitosis, and proliferation

| Sample time | | | |
|---|---|---|---|
| (hours) | Significant Cell Positions | | |
| After treatment | BrdU labeling Index | Apoptotic Index | Mitotic Index |
| 3 | 12 to 22 | None | None |
| 6 | None | None | None |
| 9 | 5 to 9 & | None | None |
| | 11 to 20 to 21 | | |
| 12 | 4 to 8 | None | None |
| 24 | 4 to 8 | None | None |
| 48 | None | None | None |

The comparisons shown in Table 9 are between treated groups versus the untreated group. The cell positions shown are the ones that are significantly different from the untreated control (P<0.05).

Group B:

In Groups 8 and 9 (Table 8), stem cell radiosensitivity was assessed. As shown in Table 8, the CG53135-05 E. coli purified product or PBS was administered one day before dosing with 1 Gy radiation. Tissues were harvested 4.5 hours after radiation dosing. There was no significant effect of CG53135-05 administration on either radiation-induced apoptosis or mitotic index. However, increased uptake in cell positions 4-8 by 12 hours and significant inhibition of proliferation were seen in mice pretreated with CG53135-05 and irradiated, consistent with the Group A results (Table 9).

### 6.4. Example 4

### Effect of CG53135 Prophylactic Administration on Mice Intestinal Crypt Survival After Radiation Injury (Study N-343)

The purpose of this study was to evaluate the efficacy of CG53135 against radiation-induced crypt cell mortality in vivo using the Clonoquant™ assay. Protein concentrations in this example were measured by Bradford assay.

Mice were weighed and then dosed with the CG53135-05 E. coli purified product (12 mg/kg) or placebo. A single injection was given, intraperitoneally (ip), 24 hours prior to irradiation. Each group of 6 animals was irradiated as per table below. For each radiation dose, the response of a drug treated group and a placebo treated group was compared.

The small intestine was removed, fixed in Carnoy's fixative, and processed for histological analysis (paraffin embedded). H&E sections were prepared following conventional protocols. For each animal, ten intestinal circumferences were analyzed, the number of surviving crypts per circumference was scored, and the average per group was determined. Only crypts containing 10 or more strongly H&E stained cells (excluding Paneth cells) and only intact circumferences, not containing Peyers patches, were scored.

The average crypt width (measured at its widest point) was also measured in order to correct for scoring errors due to crypt size difference. The correction was applied as follows:

Corrected number of crypts per circumference=Mean number of surviving crypts per circumference in treatment group X (Mean crypt width in untreated control/Mean crypt width in treated animal).

TABLE 10
Study design

| Group | Number of | | | Treatment |
|---|---|---|---|---|
| Number | Animals | Induction | Treatment | Schedule* |
| 1 | 6 males | 10 Gy | PBS | Day −1 |
| | | Day 0 | | |
| 2 | 6 males | 11 Gy, | PBS | Day −1 |
| | | Day 0 | | |
| 3 | 6 males | 12 Gy, | PBS | Day −1 |
| | | Day 0 | | |
| 4 | 6 males | 13 Gy, | PBS | Day −1 |
| | | Day 0 | | |
| 5 | 6 males | 14 Gy, | PBS | Day −1 |
| | | Day 0 | | |
| 6 | 6 males | 10 Gy | CG53135-05 E. | Day −1 |
| | | Day 0 | coli purified | |
| | | | product, 12 mg/kg, IP | |
| 7 | 6 males | 11 Gy, | CG53135-05 E. | Day −1 |
| | | Day 0 | coli purified | |
| | | | product, 12 mg/kg, IP | |
| 8 | 6 males | 12 Gy, | CG53135-05 E. | Day −1 |
| | | Day 0 | coli purified | |
| | | | product, 12 mg/kg, IP | |
| 9 | 6 males | 13 Gy, | CG53135-05 E. | Day −1 |
| | | Day 0 | coli purified | |
| | | | product, 12 mg/kg, IP | |
| 10 | 6 males | 14 Gy, | CG53135-05 E. | Day −1 |
| | | Day 0 | coli purified | |
| | | | product, 12 mg/kg, IP | |
| 11 | 6 males | Untreated | | |

Results:

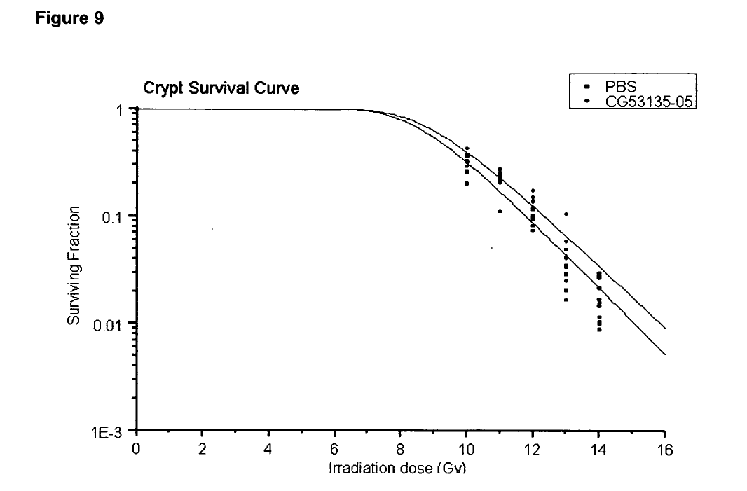

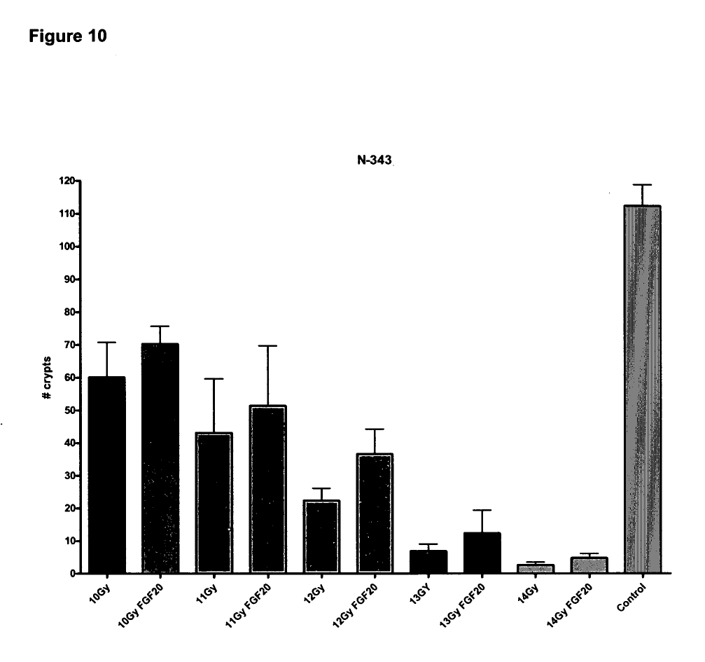

The crypt survival following prophylactic administration of the CG53135-05 E. coli purified product showed inverse correlation to the irradiation dose, that is, the smaller the radiation dose, the higher the crypt survival (FIGS. 9 and 10). Prophylactic administration of the CG53135-05 E. coli purified product significantly increased the number of crypts (P<0.001). Table 11 shows the protection factor achieved for the radiation doses following prophylactic administration of the protein (the CH53135-05 E. coli purified product). Protection factor (Table 11) represents the ratio of surviving crypt cells between treated and untreated cells. On average, 1.55 times as many cells survived irradiation dose of 12 Gy, when animals were administered with the CG53135-05 E. coli purified product prior to the radiation insult.

TABLE 11

| Radiation dose (Gy) | Protection Factor |
|---|---|
| 10 | 1.29 |
| 11 | 1.21 |
| 12 | 1.55 |
| 13 | 1.71 |
| 14 | 1.73 |

### 6.5. Example 5

### Effects of CG53135 Prophylactic Dose Schedule on Survival of Irradiated Intestinal Crypt Cells (N-375)

The objective of this study was to evaluate the ability of CG53135 to protect against radiation-induced intestinal crypt cell mortality in vivo when administered once daily for 4 days prior to irradiation. CG53135-05 E. coli purified product (12 mg/kg) or PBS was administered to BDF1 mice intraperitoneally (IP) once daily for 4 consecutive days prior to exposure to lethal radiation doses from 10-14 Gy on Day 0. The number of surviving regenerating crypt foci was measured 4 days after irradiation. Protein concentrations in this example were measured by Bradford assay.

When animals received CG53135 once daily for 4 days, an overall increase in crypt cell survival was noted when compared to PBS-treated, irradiated animals (Table 12).

TABLE 12
Intestinal Crypt Protection Factors Resulting from CG53135-05
E. coli purified product Multiple-Dose Administration
Prior to Irradiation

| | Mean Crypt | Mean Crypt Survival (#) | |
|---|---|---|---|
| Radiation | Survival (#) | CG53135-05 | Protection |
| Dose | PBS | (12 mg/kg) | Factor^{a} |
| 10 Gy | 32.7 | 32.2 | 0.98 |
| 11 Gy | 13.8 | 19.8 | 1.43 |
| 12 Gy | 6.6 | 8.9 | 1.35 |
| 13 Gy | 2.3 | 4.8* | 2.09 |
| 14 Gy | 1.7 | 1.3 | 0.76 |

^{a}Protection factor value indicates the number of surviving crypts per circumference in the CG53135-05-treated animals compared to PBS, expressed as a ratio.
*P = 0.05 versus corresponding value from PBS-treated control animals by ANOVA.
# = number of crypts.

The greatest level of radioprotection occurred following 13 Gy of radiation, with a protection factor of 2.09 (e.g., a 2-fold increase in the number of surviving crypt cells). The crypt survival curves indicated a significantly reduced sensitivity to the radiation following CG53135-05 treatment (FIG. 11). Thus, pretreatment with CG53135 for 4 consecutive days increased the overall crypt cell survival. This study indicates the use of multiple-day prophylactic dosing with CG53135-05 as a schedule with radioprotective properties.

### 6.6. Example 6

### Evaluation of Radioprotection Window (N-382)

Having established the effect of CG53135 on crypt cell radioprotection after a single day dose or multiple once daily dosing, this study evaluated the activity of CG53135 when dosed in intervals other than 24 hours prior to irradiation. CG53135-05 E. coli purified product (12 mg/kg) or PBS was administered to BDF1 mice by IP injection 6, 12, 24, 36, or 48 hours prior to exposure to a single bolus radiation dose of 13 Gy, respectively. The number of surviving regenerating crypt foci was measured 4 days after irradiation. Protein concentrations in this example were measured by Bradford assay.

Administration of a single dose of CG53135-05 E. coli purified product at 24 or 36 hours prior to irradiation offered the highest level of the intestinal crypt cell protection. These schedules resulted in increased crypt survival by 80% and 31%, respectively (Table 13). Dosing at 6, 12, or 48 h prior to irradiation resulted in dose modification factors of 0.78, 0.40, or 0.84 respectively.

TABLE 13
Intestinal Crypt Protection Factors Resulting from CG53135-05
Administration 6-48 h Prior to Irradiation

| | | | Mean | |
|---|---|---|---|---|
| | | Mean Crypt | Crypt Survival (#) | |
| Treatment | Radiation | Survival (#) | CG53135-05 | Protection |
| Schedule | Dose | PBS | (12 mg/kg) | Factor^{a} |
| −6 h | 13 Gy | 4.5 | 3.5 | 0.78 |
| −12 h | 13 Gy | 6.3 | 2.5* | 0.40 |
| −24 h | 13 Gy | 4.1 | 7.4* | 1.80 |
| −36 h | 13 Gy | 4.9 | 6.4 | 1.31 |
| −48 h | 13 Gy | 4.4 | 3.7 | 0.84 |

^{a}Protection factor value indicates the number of surviving crypts per circumference in the CG53135-05-treated animals compared to PBS-treated control animals, expressed as a ratio.
*P < 0.001 versus value from corresponding PBS-treated control animals by ANOVA.
# = number of crypts.

These results suggest that an optimal window for administration of a single dose of CG53135-05 E. coli purified product occurs from 24 to 36 hours prior to irradiation.

### 6.7. Example 7

### Effects of CG53135 Dose Schedule on Survival of Irradiated Intestinal Crypt Cells (N-416)

The objective of this study was to establishing an optimal dosing schedule of CG53135-05 administration to establish the levels of protection against radiation-induced crypt cell mortality. Protein concentrations in this example were measured by UV absorbance. CG53135-05 E. coli purified product (4 mg/kg) or phosphate-buffered saline (PBS) was administered to BDF1 male mice by intraperitoneally (IP) once daily either for 1, 2, 3, 4 or 5 consecutive days (Days −1, 0, 1, 2 and/or 3) prior to, or post-irradiation (13 Gy). The number of surviving regenerating crypt foci was measured 4 days after irradiation and the dose modification factor (DMF) were calculated.

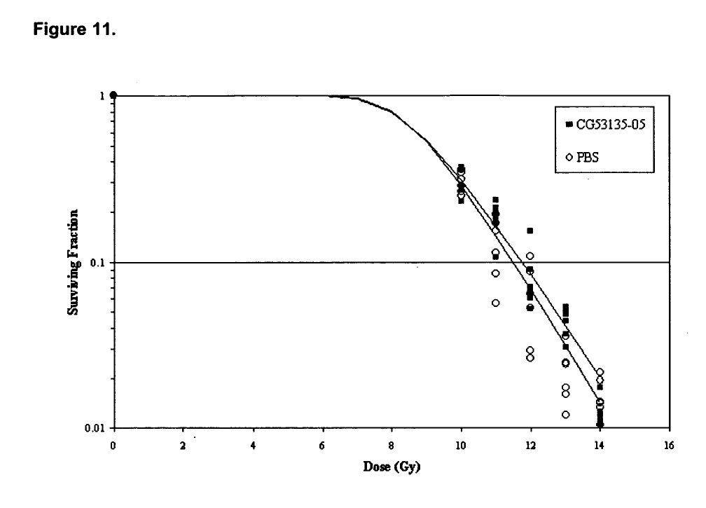

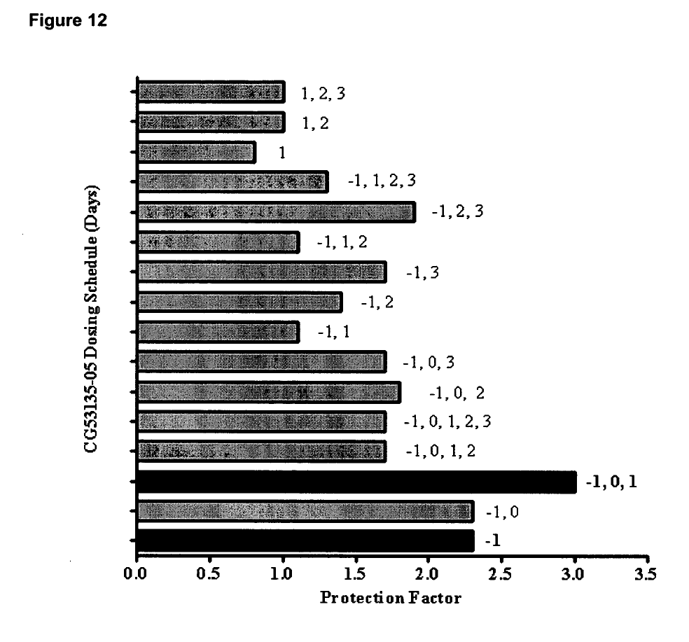

Single dose administration of CG53135-05 on Day −1 resulted in a DMF of 2.3 (FIG. 12). Administration of CG53135-05 on Days −1, 0 and 1 relative to TBI on Day 0 resulted in a DMF of 3.0 (e.g., a 3-fold increase in the number of surviving crypt cells). These data suggest that CG53135 is a GI crypt cell radioprotectant with prophylactic and intervention (treatment) properties.

### 6.8. Example 8

### Radioprotective Mechanisms of CG53135

Among the many changes that occur in a cell upon an attack of ionizing radiation, an increase of reactive oxygen species occurs via the ionization of H₂O. As this process produces the most reactive molecules within the cell, in order to reduce cellular damage, the nucleus increases transcription of enzymes that scavenge these radicals to less reactive intermediates. As CG53135 has been shown to be a radioprotectant, it is relevant to determine if treatment of cells with CG53135 upregulates any of the genes known to be involved in radioprotection in the interest of “pre-loading” the cells with oxygen radial scavenging pathways. Evaluation of the effect of CG53135 on the intricate pathways involving ROS scavengers and transcription factors will mechanistically describe the observed in vivo radioprotective effects of this agent. Thus, expression studies and survival studies were carried out at the cellular level.

Expression Studies:

To delineate the mechanism of radioprotection by CG53135, expression profile of free oxygen radical scavengers and transcription factor(s) were studied in fibroblast and endothelial cells. NIH3T3 (murine fibroblast), CCD-1070sk (human foreskin fibroblast), CCD-18Co (human colonic finbroblast), and HUVEC (human umbilical cord vascular endothelial cells) cells were transferred to basal medium containing 0.1% FBS and the indicated concentration of the CG53135-05 E. coli purified product. After 18 hours incubation, cells were harvested for total RNA using RNEasy (Qiagen, Valencia, Calif.). RNA was reverse transcribed using SuperScript First Strand Synthesis System for RT-PCR (Invitrogen, Carlsbad, Calif.) and amplified for the gene of interest using the primers and cycles indicated below.

TABLE 14

Primers for RT-PCR (Human)

| | | | |
|---|---|---|---|
| | | | No. of |
| Gene Name | Primer | SEQ ID NO | cycles |

| | | | |
|---|---|---|---|
| COX2 | 5′-TTCAAATGAGATTGTGGGAAAATTGCT-3′ | 42 | 30 |
| | 5′-AGATCATCTCTGCCTGAGTATCTT-3′ | 43 | |
| TFF3 | 5′-GTGCCGGCCAAGGACAG-3′ | 44 | 40 |
| | 5′-CGTTAAGACATCAGGCTCCAG-3′ | 45 | |
| SOD1/CuZn | 5′-TGGCCGATGTGTCTATTGAA-3′ | 46 | 30 |
| SOD | 5′-GGGCCTCAGACTACATCCAA-3′ | 47 | |
| SOD2/MnSOD | 5′-CTGGACAAACCTCAGCCCTA-3′ | 48 | 28 |
| | 5′-CTGATTTGGACAAGCAGCAA-3′ | 49 | |
| SOD3/ECSOD | 5′-TCCATTTGTACCGAAACACCCCGCTCAC-3′ | 50 | 30 |
| | 5′-CAAACATTCCCCCAAAGGAGCAGCTCTCAG-3′ | 51 | |
| Nrf2 | 5′-ATGGATTTGATTGACATACTT-3′ | 52 | 40 |
| | 5′-CTAGTTTTTCTTAACATCTGG-3′ | 53 | |
| GPX1 | 5′-AAGGTACTACTATCGAGAATGTG-3′ | 54 | 28 |
| | 5′-GTCAGGCTCGATGTCAATGGTCTG-3′ | 55 | |
| Actin | 5′-GGACTTCGAGCAAGAGATGG-3′ | 56 | |
| | 5′-AGCACTGTGTTGGCGTACAG-3′ | 57 | |

TABLE 15

Primers for RT-PCR (Mouse)

| | | | |
|---|---|---|---|
| | | | No. of |
| Gene Name | Primer | SEQ ID NO | cycles |

| | | | |
|---|---|---|---|
| MnSOD | 5′-GGGAATTCAGCGTGACTTTGGTCTTTT-3′ | 58 | 26 |
| | 5′-GCGGATCCGAGCAGGCGGCAATCTGTAA-3′ | 59 | |
| Actin | 5′-GCATCCATGAAACTACATT-3′ | 60 | |
| | 5′-CACTTGCGGTGCACGATGG-3′ | 61 | |

Results

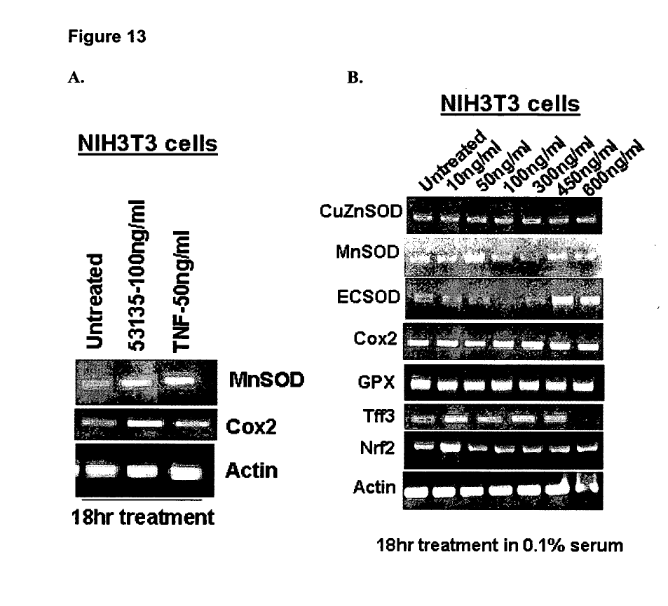

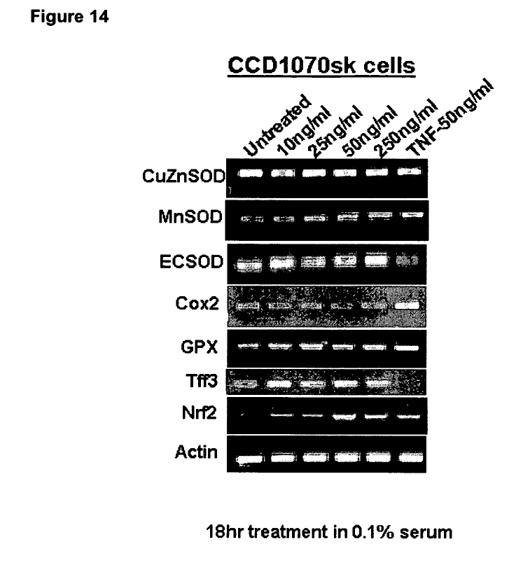

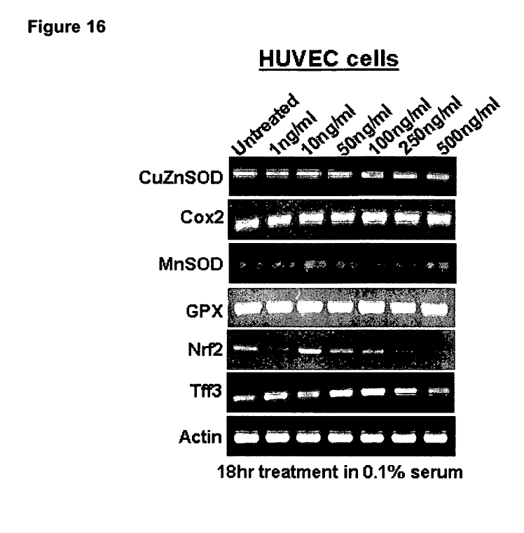

Expression results are summarized in Table 16 and shown in FIGS. 13 through 16.

TABLE 16

| Gene | NIH 3T3 | CCD 1070 | CCD 18Co | HUVEC |
|---|---|---|---|---|
| MnSOD | Increased | Moderately | Increased | No change |
| | | Increased | | |
| ECSOD | Increased | No change | Moderately | Absent |
| | | | Increased | |
| Cu, Zn-SOD | No change | No change | Increased | No change |
| Nrf2 | Increased | Increased | Increased | Increased- |
| | | | | repressed at |
| | | | | higher doses |
| COX2 | No change | No change | Increased | Moderately |
| | | | | Increased |
| Tff3 | No expression | Increased | Increased | Increased |
| GPX1 | No change | Moderately | No change | No change |
| | | Increased | | |

Among the radioprotective superoxide dismutases, MnSOD, the most radioprotective one, was found to be induced by the CG53135-05 E. coli purified product consistently among cell lines (Table 16, FIGS. 13 through 16).

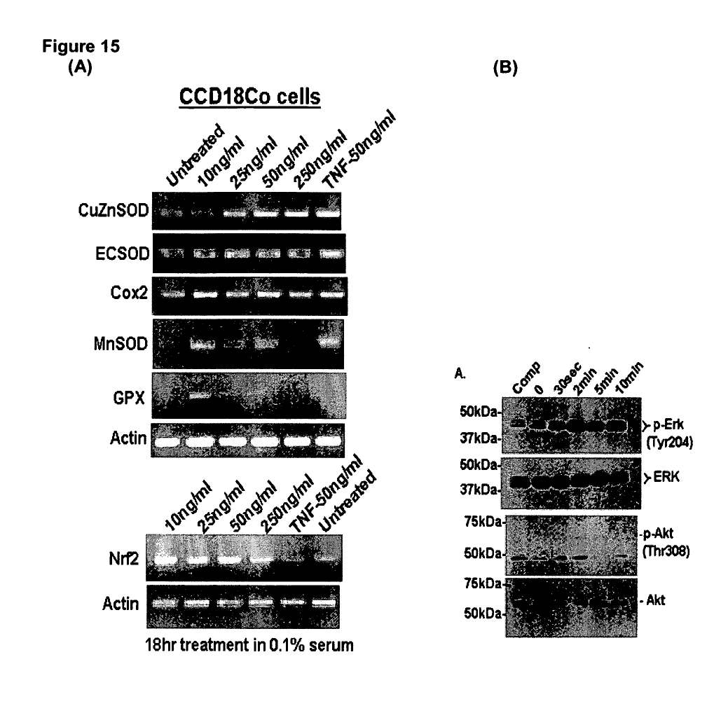

The Nrf2 transcription factor, which is involved in regulation of several antioxidants that were thought to be the “antioxidant response element” (also termed as ARE), was induced by CG53135 in all cell lines studied (Table 16, FIGS. 13 through 16). The ERK and Akt kinases are also activated by CG53135. CCD18Co human colonic fibroblasts were starved for 18 hours in basal media containing 0.1% BSA or left in complete serum (“Comp”) then stimulated with 100 ng/ml FGF-20 and harvested at the time points indicated. Lysates were immunoblotted for human ERK or Akt or their indicated phosphorylated counterparts. Both ERK and Akt kinases were active by 2 minutes of treatment with the CG53135-05 E. coli purified product (FIG. 15). The activation of these kinases, particularly Akt, has been associated with radioprotective events.

It is well established that one mechanism by which cells are protected from ionizing radiation is through the induction of oxygen radical scavenging pathways. The gene expression studies detailed herein indicate that this may be one of the pathways by which CG53135 modulates radioprotection. Furthermore, one of the primary target organs of total body irradiation (TBI) is the gastrointestinal tract. The data disclosed herein show that (1) the most responsive of all cell lines studied was a gastrointestinal fibroblast (CCD-18co); and (2) a well-characterized intestinal radioprotectant, Tff3, was strongly upregulated by CG53135. Considering that other radioprotectants known in the art strictly affect bone marrow survival and no other compartments, it is important to note that CG53135 is active in a tissue that is as equally affected as the hematopoietic stem cells, but no less important to the survival of the animal.

Survival Studies:

Cells that receive a certain dose of radiation will have to brace against the onslaught of ionized radicals, repair the damage that the radicals perform or delay the onset of apoptosis in the face of irreperable harm, or, likely, a combination of all these mechanisms. Each of these pathways is thus important for the ultimate survival of a cell and its ability to proliferate. Cell survival was assessed by a clonogenic assay in which surviving cells can form colonies in vitro after irradiation.

Clonogenic assays were performed using CCD-18co cells, FaDu human squamous cell carcinoma cells, IEC6 and IEC18 rat colon crypt cells, and NIH 3T3 mouse fibroblast cells, to assess the effect of CG53135 on radiation protection. Cell culture conditions were as follows: NIH 3T3 cells were grown in DMEM+10% Bovine serum+50 μg/ml Pennicilin/Streptomycin; IEC6 and IEC18 cells were grown in DMEM+10% FBS+0.1 U/ml Insulin+50 μg/ml Pennicilin/Streptomycin; FaDu cells were grown in MEM+10% FBS+1 mM Sodium Pyruvate+50 μg/ml Pennicilin/Streptomycin+Non-essential amino acids. Cells were plated at a density of 5×10⁵ per 10 cm dish (NIH3T3) or 5×10⁵ per well of a 6-well dish (IEC18, IEC6, FaDu) and allowed to attach. Cells were then treated with the CG53135-05 E. coli purified product at doses of 10 or 100 ng/ml (IEC18, IEC6, FaDu) or at 50 and 200 ng/ml (NIH 3T3) in basal media containing 0.1% serum (IEC18, IEC6, FaDu) or 1% serum (NIH 3T3) and incubated for 16 hours (IEC18, IEC6, FaDu) or 1 hour (NIH 3T3). Cells were then irradiated using a Faxitron X-ray irradiator (Wheeling, Ill.) fitted with a 0.5 mm aluminum filter at 2.5, 5, 7.5, 10, 12.5 and 15 Gy at 130 kVp, resulting in a radiation rate of 50 cGy/min. Immediately after irradiation, cells were trypsinized and plated in duplicate at densities of NIH 3T3: 250, 500, 1000, 2000, 5000 and 10,000 cells per 60 mm dish; FaDu, IEC18 and IEC6: 500, 2500 cells per well of a 6-well dish. Cells were grown in complete growth medium for 1-2 weeks until colonies of average diameter of 2 mm, after which the colonies were stained with crystal violet and counted.

Results:

The number of surviving untreated or CG53135-treated cells was plotted as a function of radiation dose, giving rise to survival curves. The slopes of different parts of the survival curves describe different properties of radiation cell killing and can be described as follows:

D0 is the slope of the curve between the final two points, indicating speed of cell killing at the higher doses of radiation. The value is interpreted to indicate the amount of radiation required to reduce the fraction of surviving cells by 37% of the previous value on the graph. A smaller number indicates a more rapid rate of cell killing.

D1 is the slope of the curve between the first two points, indicating the speed of cell killing at the lower doses of radiation. The value is interpreted as the amount of radiation required to reduce the fraction of surviving cells by 37% of the previous value on the graph. A smaller number indicates a more rapid rate of cell killing.

Dq is the width of the shoulder of the curve before an exponential decrease in cell survival is seen. This is essentially the threshold amount of irradiation required before an incidence of cell killing is seen. A larger Dq value indicates that the cells are completely protected at the lower doses of radiation.

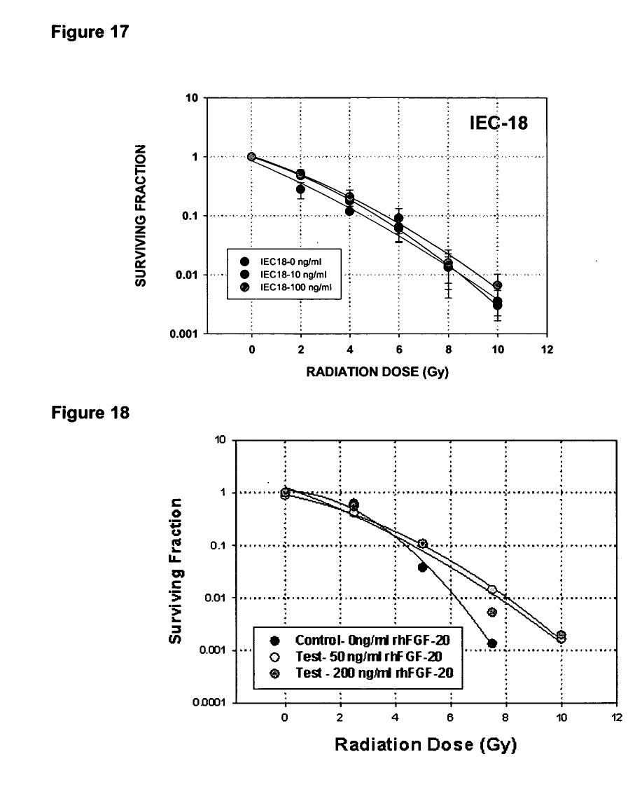

The effect of CG53135 treatment on survival of irradiated IEC18 cells is shown in FIG. 17. While the D0 and D1 values showed no obvious treatment-related trends, the Dq value indicates that IEC18 cells treated with CG53135 are more protected from killing at the lower doses of radiation compared to untreated cells (shoulder of survival curves of cells treated with 10 ng/ml and 10 ng/ml CG53135 is broader). Thus, treatment of IEC18 cells with the CG53135-05 E. coli purified product results in cell killing at a higher dose of radiation compared to untreated cells, indicating that CG53135 acts as a radioprotectant in these cells.

The effect of CG53135 treatment on survival of irradiated NIH 3T3 cells is shown in FIG. 18. The D0 values for NIH 3T3 cells treated with 50 ng/ml or 200 ng/ml CG53135 appear larger than the D0 value for untreated cells, and the difference approaches significance for the 50 ng/ml dose. These results suggest that CG53135 may act as a radioprotectant, promoting survival of NIH 3T3 cells at the higher doses of radiation. Furthermore, the D1 value for cells treated with 100 ng/mL CG53135 is smaller than for untreated cells, indicating a slower rate of the cell death at lower doses of radiation. No trend in Dq values of the survival curves could be determined, largely due to the variation of survival in untreated cells.

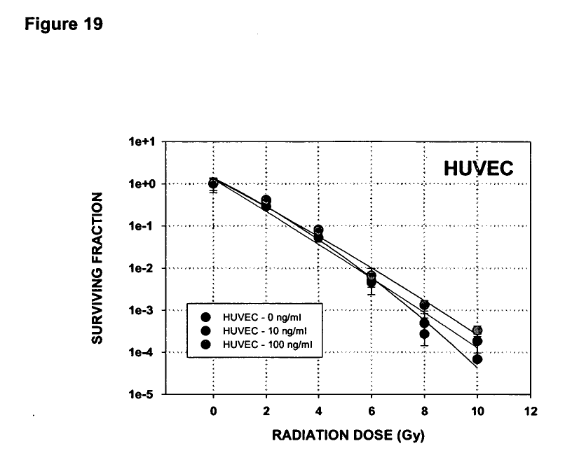

The effect of CG53135 treatment on survival of irradiated HUVEC cells is shown in FIG. 19. The D0 value for cells treated with 100 ng/mL CG53135 is higher than that for untreated HUVEC cells or cells treated with 10 ng/ml CG53135, indicating a slower rate of cell death at higher radiation doses. In addition, the Dq value for cells treated with 100 ng/mL CG53135 suggests that there is a slower rate of cell death at low doses of radiation compared to untreated HUVEC cells or cells treated with 10 ng/ml CG53135. No obvious effects of treatment of CG53135 on D1 values were observed. Thus, treatment of HUVEC cells with 100 ng/ml CG53135-05 E. coli purified product provides a significant decrease in the speed of cell killing at the high dose of radiation. The HUVEC cells treated with 100 ng/ml CG53135-05 E. coli purified product also appeared to be more protected from killing at low doses of radiation compared to untreated cells.

In contrast, irradiated FaDu and IEC6 cells did not show any obvious trends in D0, Dq or D1 values as a result of CG53135 administration.

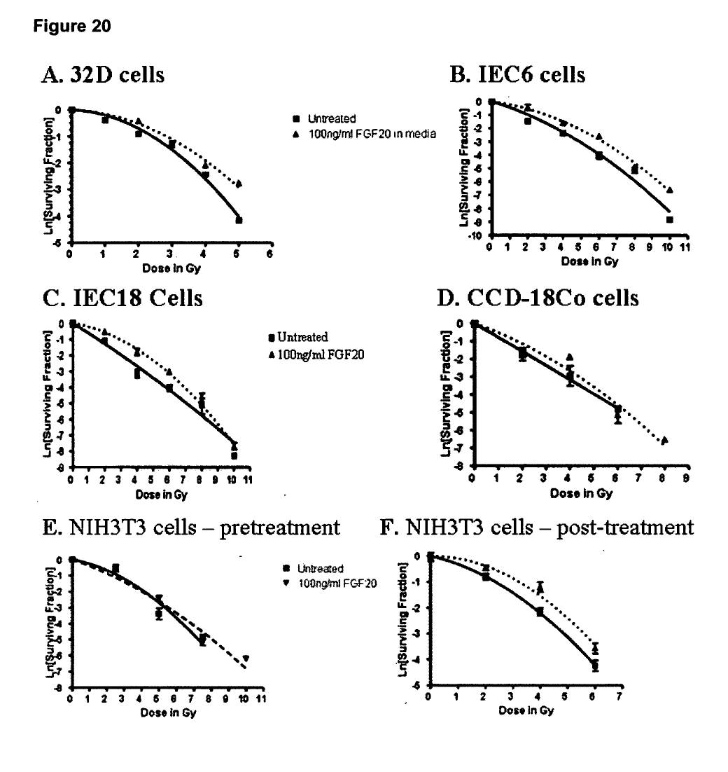

In another study (L-411 and L-432), post-radiation cell survival was examined in 7 cell lines that were representative of different cell types in each layer of intestinal mucosa: epithelium (IEC6 and IEC18, rat intestinal epithelia), mesenchyme (NIH3T3, mouse fibroblast; CCD-18Co, human colonic fibroblast), and hematopoietic (32D, murine hematopoietic cell line) using a clonogenic assay (as described above). Cells were irradiated, plated in complete growth media with or without 100 ng/ml CG53135-05 E. coli purified product, and allowed to form colonies for 10-14 days. The data were plotted and analyzed for rate of cell killing at high doses (D0) and low doses of radiation (D1, Dq) (FIG. 20, Table 17).

TABLE 17
Survival Response Parameters for Cells

| Cell Type | Treatment | D₀ | D_{q} | D₁ |
|---|---|---|---|---|
| 32D Cells | Untreated | 0.60 | 2.60 | 20.38 |
| (Hematopoietic) | 100 ng/mL CG53135-05 | 0.71 | 3.06 | 21.45 |
| | Post-irradiation | | | |
| IEC6 Cells | Untreated | 1.03 | 1.98 | 2.50 |
| (Epithelial) | 100 ng/mL CG53135-05 | 1.03 | 3.32 | 5.89 |
| | Post-irradiation | | | |
| IEC18 Cells | Untreated | 1.27 | 0.68 | 1.61 |
| (Epithelial) | 100 ng/mL CG53135-05 | 0.95 | 3.15 | 5.88 |
| NIH3T3 Cells | Untreated | 0.88 | 2.87 | 5.22 |
| (Mesenchymal) | 100 ng/mL CG53135-05 | 1.18 | 2.15 | 2.72 |
| | Prior to irradiation | | | |
| | 100 ng/mL CG53135-05 | 0.75 | 3.46 | — |
| | Post-irradiation | | | |
| CCD-18Co | Untreated | 1.23 | 0.13 | 1.29 |
| (Mesenchymal) | 100 ng/mL CG53135-05 | 0.95 | 1.68 | 2.13 |
| | Post-irradiation | | | |
| U2OS | Untreated | 0.77 | 0.49 | 1.02 |
| (Bone) | 100 ng/mL CG53135-05 | 0.79 | 0.76 | 1.21 |
| | Prior to irradiation | | | |
| | 100 ng/mL CG53135-05 | 0.80 | 1.60 | 2.03 |
| | Post-irradiation | | | |
| Saos2 | Untreated | 0.74 | 0.95 | 1.32 |
| (Bone) | 100 ng/mL CG53135-05 | 0.74 | 1.37 | 1.73 |
| | Post-irradiation | | | |

The D₁ and D_{q} parameters are indicative of the rate of cell killing at low doses of radiation, whereas the D₀ parameter reflects the rate of killing at high doses of radiation. An increase in these parameters in CG53135-05-treated cells as compared to untreated indicates a protective effect.

Significant protection was observed the 32D, NIH3T3, IEC18, IEC6 and U2OS cell lines, while more modest protection was seen in the CCD18Co and Saos lines (FIG. 20, Table 17). Treatment of U2OS and NIH3T3 cells with FGF-20 after irradiation was more efficacious than pretreatment.

In summary, these results indicate that the CG53135-05 E. coli purified product has a protective effect against radiation in vitro.

### 6.9. Example 9

### Effect of CG53135 on Cytokine Release

Cytokines are important cell signaling proteins mediating a wide range of physiological responses. Ionizing radiation can trigger a series of changes in gene expression and cytokine profiles. The aim of this study was to evaluate the cytokine profile upon CG53135 treatment in cell culture over a time course.

BioPlex cytokine assays, which are multiplex bead based assays designed to quantitate multiple cytokines from tissue culture supernatants, were used for detecting the cytokines. The principle of the assay is similar to a capture sandwich immunoassay. NIH 3T3 cells were plated in a 96 well plate. The cells were washed with DMEM+0.1% Calf Serum (SFM). The CG53135-05 E. coli purified product, at 10 ng/ml or 100 ng/ml, was added to the cells. The cell supernatant was collected after 30 minutes, 1 hour, 2 hours, 4 hours, 6 hours, and 24 hours. Fifty ng of TNF was used as a positive control. Bioplex 18-Plex Cytokine Assay (BioRad Laboratories Inc, CA) was performed following the procedure of the manufacturer.

Results:

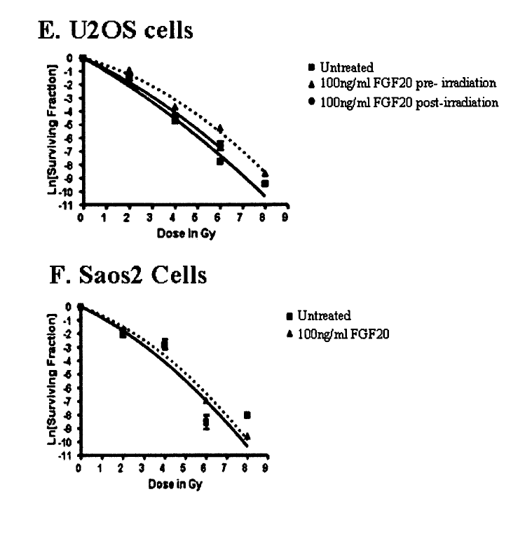

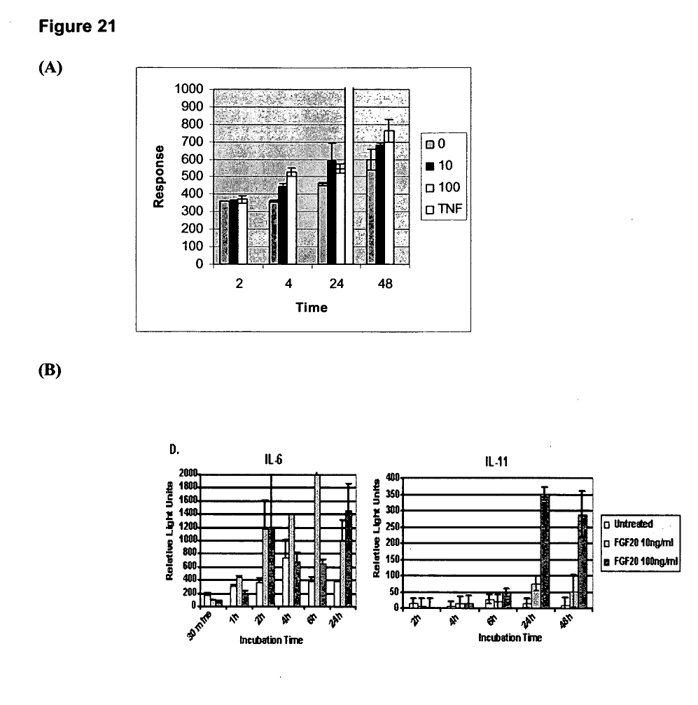

FIG. 21 shows the effect of the CG53135-05 E coli purified product on Mo KC release. Mo KC is also known as the chemokine CXCL1 (which also has been described as Gro1, Melanoma growth stimulatory activity (MSGA) or neutrophil-activating protein-3 (NAP3)). It functions as a chemoattractant for neutrophils, signalling through the CXCR1 receptor. It has also been implicated in the response to whole body irradiation, raising the possibility that it possesses radioprotective qualities of its own (see Radiat. Res. 160:637-46, 2003). FIG. 21 shows a consistent dose (p=0.0085) and time dependent increase (p=4.6×10⁻⁶) in the measured response. In addition both the concentrations of the CG53135-05 E. coli purified product showed significantly higher response than the control (no CG53135).

IL-6 and IL-11 expression in response to CG53135 treatment was also examined. Both IL-6 and IL-11 have recently been implicated in the response to total body irradiation. In addition, IL-11 has been used as an agent to combat thrombocytopenia following chemo- or radiotherapy. CCD18Co cells were incubated with 100 ng/ml CG53135 in basal media containing 0.1% BSA for the indicated time periods. Conditioned media was removed and analyzed for IL-6 and IL-11 concentration by Luminex or ELISA respectively. FIG. 21B shows that IL-6 and IL-11 cytokines are induced upon exposure to the CG53135-05 E. coli purified product in vitro.

Additional experiments can be performed to determine CG53135-05 in combination with CXCL1 acts synergistically in radioprotection, both in vitro and in vivo. Furthermore, induction of other cytokines (e.g., IL-2, IL4, IL-5, IL-6, IL-8, IL-10, MCP-1, GM-CSF, RANTES) can also be tested, as a skilled person in art would recognize, in the presence of CG53135-05 in different cell lines (e.g., HUVEC, CCD-18, NIH3T3).

### 6.10. Example 10

### Measurement of Scavengers of Reactive Oxygen Intermediates After Radiation Exposure

CM-H₂DCFDA Method

Cells with increased reactive oxygen species, as a first step, upregulate the Superoxide Dismutases—Cu, Zn-SOD, Mn-SOD, and Extracellular-SOD to scavenge the superoxide radical to hydrogen peroxide. Activity of these enzymes can be indirectly measured by their byproduct of H₂O₂ using an acetoxymethyl ester. A derivative of this class, 5-(and -6)-chloromethyl-2′,7′-dichlorodihydrofluorescein diacetate, also known as CM-H₂DCFDA, is efficiently retained within the cell and fluoresces green when oxidized by H₂O₂.

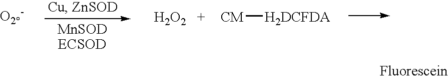

IEC18 (rat intestinal epithelial) and CCD-18Co (human colonic fibroblast) cells were plated to 60 mm dishes at a density of 1×10⁵ cells per dish. After attachment, the cells were switched to medium containing 0.1% serum and the indicated dose of CG53135. After 18 hours of incubation, the cells were then irradiated at 2 or 4 Gy with X-rays using a Faxitron X-irradiator (Wheeling, Ill.), followed by incubation with 5 mM CM-H₂DCFDA (Molecular Probes, Eugene, Oreg.) for 15 minutes. The cells were then washed, trypsinized, and analyzed on a Becton Dickinson FACSCalibur (San Jose, Calif.) on the FL1 channel.

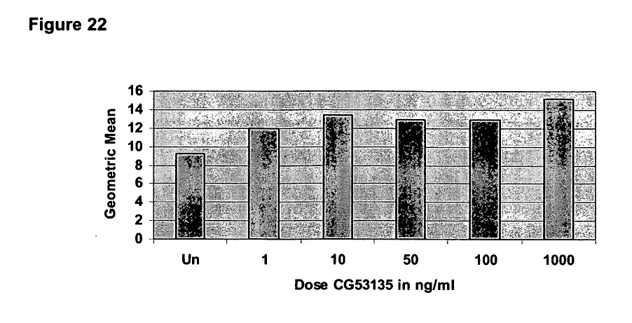

Results indicate that IEC18 and CCD18Co cells possess increased intracellular H₂O₂ after treatment with the CG53135-05 E. coli purified product in a dose responsive manner (FIGS. 22, 23 and 24). This is believed to be due to enhanced expression of Superoxide dismutases, predominantly MnSOD induced by the CG53135-05 E. coli purified product.

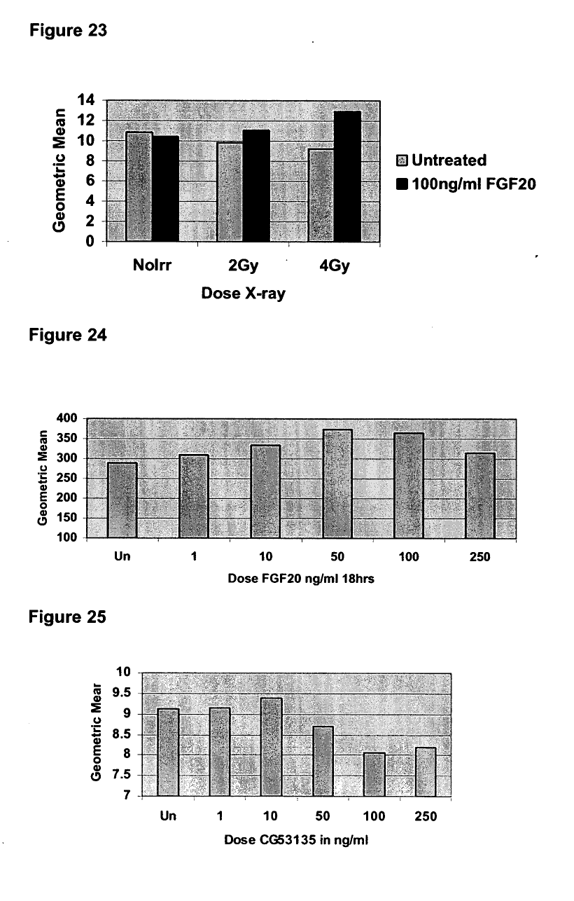

Production of intracellular H₂O₂ by the CG53135-05 E. coli purified product in IEC18 cells is enhanced with increasing dose of ionizing radiation (FIG. 23). This result reflects the increased production of more reactive oxygen species such as superoxide and hydroxyl by radiation, thus increasing substrate for the Superoxide Dismutases induced by the CG53135-05 E. coli purified product.

Red CC-1 Method

Upon ionizing radiation exposure, cells accumulate reactive oxygen species as a result of radiation ionizing H₂O to the hydroxyl radical (OH), superoxide (O₂⁻) or hydrogen peroxide (H₂O₂). As these ions in abundance can have deleterious effects on the cell, pathways are upregulated to scavenge these molecules to more stable variants. It is hypothesized that CG53135 may protect the cell from ionizing radiation damage by upregulating pathways that reduce the redox capacity of the cytosol. A dye called Redox Sensor 1 (Red CC-1) can monitor the redox level of the cytosol upon oxidation by changing to a red fluorescent agent that can be measured by FACS on the FL2 channel.

Cytosolic Reactive Oxygen Species+Red CC-1 ? Oxidized Red CC-1 IEC18 (rat intestinal epithelial) and CCD-18Co (human colonic fibroblast) cells were plated to 60 mm dishes at a density of 1×10⁵ cells per dish. After attachment, the cells were switched to medium containing 0.1% serum and the indicated dose of the CG53135-05 E. coli purified product. After 18 hours of incubation, the cells were then irradiated at 2 or 4 Gy with X-rays using a Faxitron X-irradiator, followed by incubation with 5 mM Red CC-1 (Molecular Probes, Eugene, Oreg.) for 15 minutes. The cells were then washed, trypsinized, and analyzed on a Becton Dickinson FACSCalibur on the FL2 channel.

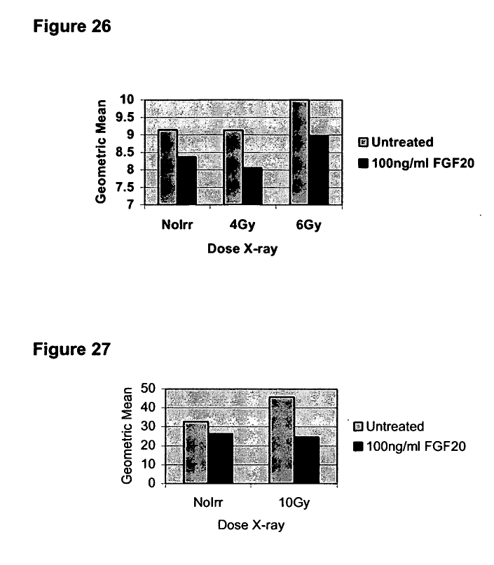

Results: IEC18 and CCD18Co cells were found to possess decreased cytosolic redox potential after treatment with CG53135-05 in a dose responsive manner as shown in FIGS. 25, 26, and 27. The data shown herein is believed to be the result of enhanced expression of superoxide dismutases induced by the CG53135-05 E. coli purified product, which scavenge the more reactive species of superoxide and hydroxyl radicals. Also, the CG53135-05 E. coli purified product is shown to increase expression of a key antioxidant-controlling transcription factor, Nrf2, which may contribute to this reduction in reactivity in the cytosol in other ways.

### 6.11. Example 11

### In vitro Radioprotection of the Myeloid Cell Line 32D

The radioprotection effect of CG53135 in myeloid cells was also studied by in vitro experiment using the myeloid cell line 32D. 32D cells were irradiated at 0, 1, 2, 3, 4 or 5 Gy then plated in methylcellulose-containing growth media including 10 ng/ml IL-3 with or without 100 ng/ml CG53135-05 E. coli purified product. Cells were allowed to form colonies for 10 days and were then scored. FIG. 28 shows increased survival of 32D cells upon exposure to the CG53135-05 E. coli purified product. The cell survival is plotted by the natural Log of the surviving fraction, and a linear quadratic equation was used to obtain the curve. The qualities of the curve, i.e., D1, Dq and D0, indicating at what points radioprotection are observed, were derived using the methods described in Hall et al., Radiat. Res. 114(3):415-424 (1988), the description of which is incorporated herein by reference in its entirety. An increase in the Dq value, indicating the amount of radiation required for cell death to start exponentially (or the width of the “shoulder” of the curve at low radiation doses) from 2.10 Gy to 2.79 Gy was observed.

### 6.12. Example 12

### Effect of CG53135 on Repopulation of Thymus Following Bone Marrow Ablation and Subsequent Bone Marrow Transplant

Long-term effects of CG53135 specifically in the thymus microenvironment on reconstitution of the immune system were also examined. Protein concentrations in this example were measured by UV absorbance. The CG53135 E. coli purified product was tested in a bone marrow ablation and transplantation model and repopulation of the thymus with thymocytes was examined. Mice were irradiated with 9 Gy to ablate the bone marrow, and subsequently underwent bone marrow transplantation. Prior to this, one group of mice was dosed with 16 mg/kg (UV) CG53135 (IP), once daily on days −3, −2, −1, 0 and +1 relative to the day of bone marrow ablation. Thirty days after bone marrow transplantation, the thymi of both untreated and treated mice were harvested and thymocytes collected. Cells were counted (A) as well as stained (B) for the T-cell specific markers CD4 and CD8.

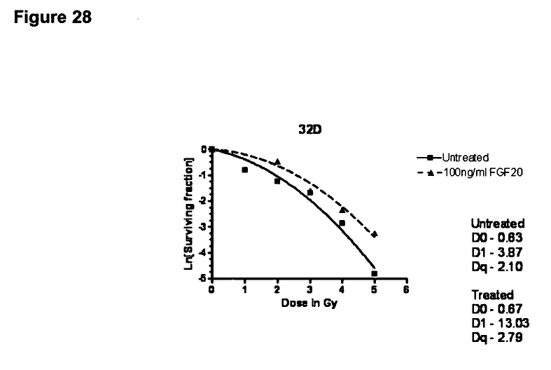

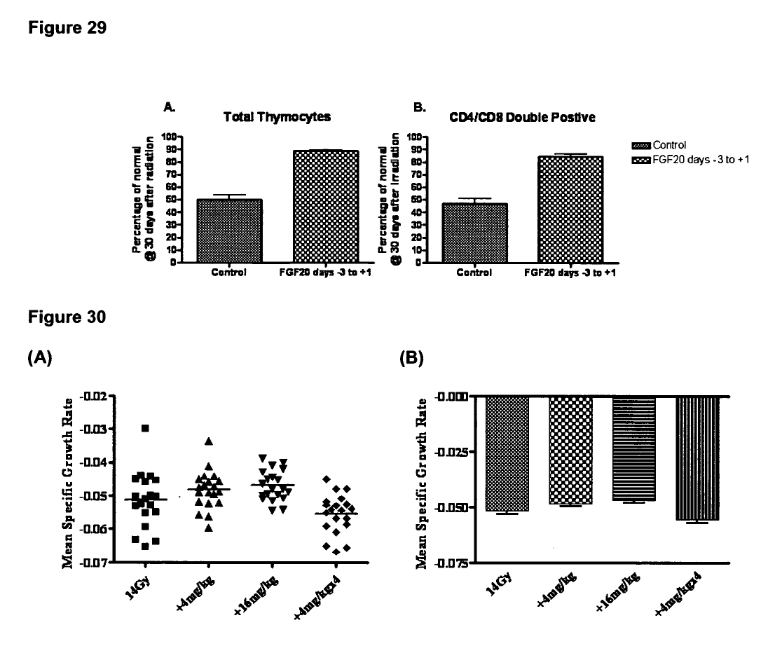

FIG. 29 shows that the total thymocyte cell population, as well as mature CD4/CD8 positive T-cells within the thymus, was significantly increased in animals treated with the CG53135-05 E. coli purified product (p=0.00003).

### 6.13. Example 13

### Determination of Optimal Dose and Schedule with IV Dosing of CG53135 in Mice

In previous example, an effect at higher doses (16 mg/kg UV) than previously used in the rodent survival study (12 mg/kg Bradford) was observed. Furthermore, in previous experiments intraperitoneal (IP) dosing of CG53135 was used. Administration of CG53135 in non-human primates is tested using intravenous (IV) delivery, which is a preferred administration route. The LD₅₀ for bone marrow failure due to total body irradiation in mice is ˜7 Gy. Lower doses of radiation have been shown to result in hematologic damage, with numbers of granulocytes, lymphocytes, and erythrocytes decreasing to nadirs by 10 days after irradiation. However, the cell numbers rebound by 30 days after irradiation. Such a parabolic effect can allow one to monitor the effectiveness of a compound on hematopoietic protection from damaging radiation. Mice are dosed with 0, 16 or 24 mg/kg IV (UV) of the CG53135-05 E. coli purified product on −3, −2, −1, 0 and +1 days revolving around the day of irradiation with 60 mice per group. The day −3, −2, −1, 0 and +1 CG53135 dosing schedule has provided the most consistent results in both the survival study and thymus reconstitution models described in previous sections, supra. Twenty mice from each group are irradiated on day 0 with 3, 4, or 5 Gy X-ray at a dose rate of 1 Gy/minute. On days 4, 12, 20 and 30 post-irradiation, five animals from each group are sacrificed to collect peripheral blood for analysis of circulating blood cells (CBC, hematocrit, etc.) and to obtain bone marrow from the femur for plating in differentiating methylcellulose media. This study should identify the optimal dose of the CG53135-05 E. coli purified product at which clinical benefit can be demonstrated in rodents, and the optimal dose to test in primate studies. The outline of the study is as follows (protein concentrations in this example were measured by UV absorbance):

TABLE 18

| Group | Number of | | | Treatment | Volume |
|---|---|---|---|---|---|
| Number | Animals | Irradiation | Treatment | Schedule | (mL) |
| 1 | 5 Males | None | None | None | None |
| 2 | 20 Males | 3Gy | None | Days −3, −2, −1, 0 | Based on |
| | | | | and +1 | Body Weight |
| 3 | 20 Males | 4Gy | None | Days −3, −2, −1, 0 | Based on |
| | | | | and +1 | Body Weight |
| 4 | 20 Males | 5Gy | None | Days −3, −2, −1, 0 | Based on |
| | | | | and +1 | Body Weight |
| 5 | 20 Males | 3Gy | CG53135-05 E. coli | Days −3, −2, −1, 0 | Based on |
| | | | purified | and +1 | Body Weight |
| | | | product, 8 mg/kg, | | |
| | | | IV, once | | |
| | | | daily | | |
| 6 | 20 Males | 4Gy | CG53135-05 E. coli | Days −3, −2, −1, 0 | Based on |
| | | | purified | and +1 | Body Weight |
| | | | product, 8 mg/kg, | | |
| | | | IV, once | | |
| | | | daily | | |
| 7 | 20 Males | 5Gy | CG53135-05 E. coli | Days −3, −2, −1, 0 | Based on |
| | | | purified | and +1 | Body Weight |
| | | | product, 8 mg/kg, | | |
| | | | IV, once | | |
| | | | daily | | |
| 8 | 20 Males | 3Gy | CG53135-05 E. coli | Days −3, −2, −1, 0 | Based on |
| | | | purified | and +1 | Body Weight |
| | | | product, 16 mg/kg, | | |
| | | | IV, once | | |
| | | | daily | | |
| 9 | 20 Males | 4Gy | CG53135-05 E. coli | Days −3, −2, −1, 0 | Based on |
| | | | purified | and +1 | Body Weight |
| | | | product, 16 mg/kg, | | |
| | | | IV, once | | |
| | | | daily | | |
| 10 | 20 Males | 5Gy | CG53135-05 E. coli | Days −3, −2, −1, 0 | Based on |
| | | | purified | and +1 | Body Weight |
| | | | product, 16 mg/kg, | | |
| | | | IV, once | | |
| | | | daily | | |
| 11 | 20 Males | 3Gy | CG53135-05 E. coli | Days −3, −2, −1, 0 | Based on |
| | | | purified | and +1 | Body Weight |
| | | | product, 24 mg/kg, | | |
| | | | IV, once | | |
| | | | daily | | |
| 12 | 20 Males | 4Gy | CG53135-05 E. coli | Days −3, −2, −1, 0 | Based on |
| | | | purified | and +1 | Body Weight |
| | | | product, 24 mg/kg, | | |
| | | | IV, once | | |
| | | | daily | | |
| 13 | 20 Males | 5Gy | CG53135-05 E. coli | Days −3, −2, −1, 0 | Based on |
| | | | purified | and +1 | Body Weight |
| | | | product, 24 mg/kg, | | |
| | | | IV, once | | |
| | | | daily | | |

### 6.14. Example 14

### Determination of Optimal Dose and Schedule for IV Administration of CG53135 in Nonhuman Primates

The optimal dose and schedule for IV administration of CG53135 can also be tested in a nonhuman primate radiation model for hematopoietic injury. Historically, studies involving radiation protection have utilized the male rhesus monkey, Macaca mulatta. Five groups of five animals are used. Based on activity in the rodent model, a dose of the CG53135-05 E. coli purified product is chosen and the equivalent dose in monkey is identified. This optimal dose is bracketed with one higher and one lower dose. The schedule of CG53135 dosing (day −3, −2, −1, 0, +1) is identical to the schedule used in the rodent study. On day 0, animals undergo total body irradiation at a dose of 4 Gy, as this dose has demonstrated hematopoietic depression in this species of monkey. Monkeys are unilaterally irradiated in Lucite restraining chairs with 250 kVp X-rays at a dose rate of 13 cGy/min. Complete blood counts with differential are performed every day for 60 days with peripheral blood, which is obtained from the saphenous vein. Conditions such as neutropenia and thrombocytopenia are of particular interest. Approximately 2 ml of heparinized bone marrow is aspirated from sedated animals on days 7, 14, 21 and 46 post-irradiation. Mononuclear cells are isolated by density centrifugation and placed in methylcellulose-based media containing cytokines (GM-CSF, erythropoietin, IL-3, c-kit, etc.) to identify the presence and health of myeloid progenitors. The study design is shown in Table 19:

TABLE 19

| | Number | | | | |
|---|---|---|---|---|---|
| Group | of | | | Treatment | Volume |
| Number | Animals | Irradiation | Treatment | Schedule | (mL) |
| 1 | 5 Males | None | Autologous serum | None | None |
| 2 | 5 Males | 4Gy | Autologous serum | Days −3, −2, −1, 0 | Based on |
| | | | | and +1 | Body Weight |
| 3 | 5 Males | 4Gy | CG53135-05 E. coli | Days −3, −2, −1, 0 | Based on |
| | | | purified product 8 mg/kg | and +1 | Body Weight |
| | | | (UV), IV, once daily | | |
| 4 | 5 Males | 4Gy | CG53135-05 E. coli | Days −3, −2, −1, 0 | Based on |
| | | | purified product 16 mg/kg | and +1 | Body Weight |
| | | | (UV), IV, once | | |
| | | | daily | | |
| 5 | 5 Males | 4Gy | CG53135-05 E. coli | Days −3, −2, −1, 0 | Based on |
| | | | purified product 24 mg/kg | and +1 | Body Weight |
| | | | (UV), IV, once | | |
| | | | daily | | |

Clinical support, including antibiotics, fresh irradiated whole blood and fluids, is provided to the animals throughout the experiment as needed. Antibiotics (i.e., Gentamicin, Baytril) are administered after irradiation until the animal maintains a white blood cell count greater than or equal to 1000/μl for 3 straight days and an absolute neutrophil count of greater than or equal to 500/μl. When a platelet count is below 20,000/μl and the hematocrit is below 18%, fresh irradiated whole blood is administered at approximately 30 ml/transfusion. At the end of the study animals are euthanized, and bone marrow, spleen, liver and jejunum are harvested for histopathological examination. This study should determine the optimal dose for this schedule and route of administration for nonhuman primates, with the objectives of demonstrating clinical benefit of CG53135 treatment in a second animal species and predicting the optimal dose for humans.

### 6.15. Example 15

### Direct Effect of CG53135 on Hematopoietic and Mesenchymal Progenitors From Irradiated Human Bone Marrow

Fresh bone marrow obtained from normal human donors is purchased from a commercial vendor. Mononuclear cells are isolated by density centrifugation, then transferred to tissue culture dishes containing Iscove's modified Dulbecco's medium with or without CG53135-05 E. coli purified product at concentrations of 10, 100 and 500 ng/ml and incubated for one hour. Cells will then be irradiated with 0, 1, 2 or 4 Gy using an X-ray radiation source. Irradiated cells are then transferred at the appropriate densities to dishes containing methylcellulose-based media containing hematopoietic stem cell factors c-kit and IL-3 as well as the factors GM-CSF and erythropoietin to allow differentiation along the granulocytic and erythrocytic lineages, respectively. In one half of the dishes, CG53135-05 E. coli purified product is added to the media at a concentration of 100 ng/ml. This dosing schedule will be used to determine whether CG53135 is active in pre-treatment, post treatment, or both with respect to irradiation as follows:

TABLE 20

| | |
|---|---|
| Irradiated cells - | Irradiated cells - |
| no CG53135 in growth media | Pre-treated with 10/100/500 ng/ml |
| No treatment | CG53135- |
| | no CG53135 in growth media |
| | Pre-treatment |
| Irradiated cells - | Irradiated cells - |
| no CG53135 pretreatment - | Pre-treated with 10/100/500 ng/ml |
| CG53135 at 100 ng/ml in growth | CG53135- |
| media | CG53135 at 100 ng/ml in growth |
| Post-treatment | media |
| | Pre- and post-treatment |

Cells are grown for 10 days and are scored for colonies representative of erythroid lineage (BFU-E, CFU-E), granulocytic/macrophage lineage (CFU-GM), or a progenitor of both lineages (CFU-GEMM). A subset of irradiated cells are placed in media consisting of Iscove's modified Dulbecco's media, 25% equine serum and hydrocortisone to allow the growth of stromal cells, and are subjected to a similar treatment protocol as described above. After 2 weeks, colonies are stained and scored for morphologies resembling fibroblasts or mixed fibroblast/adipocyte colonies.

### 6.16. Example 16

### Indirect Effect of CG53135 on Bone Marrow Repopulation Through Stimulation of Bone Marrow Stromal Response

CG53135 imparts a protective or proliferative effect on the bone marrow stem cells by, among other mechanisms, stimulating fibroblasts within the marrow stroma to secrete factors that facilitate the health and proliferative capacity of hematopoietic stem cells. To determine what factors are secreted by the stroma via CG53135 treatment, human marrow stromal cells are purchased and plated out in 96-well dishes. The CG53135-05 E coli purified product is added to the medium at concentrations of 10, 100 and 500 ng/ml for time periods of 0, 1, 4, 8, 24, and 48 hours. After incubation, the conditioned media is removed and analyzed for cytokine release using a Bio-Rad Bio-Plex to screen for 17 human cytokines, as well as ELISA to test for additional known radioprotectants such as IL-1a and IL-11, and known stimulants of hematopoietic expansion such as IL-3 for stem cells and IL-7 for T-cells. CG53135 has been demonstrated to have a proliferative effect on mouse and human fibroblasts (Jeffers et.al., 2001). Bone marrow stromal cells are tested for their ability to replicate in response to CG53135 treatment. Cells are starved in growth factor-free medium for 24 hours, then stimulated with 10, 100 or 500 ng/ml CG53135 for 1, 3, and 5 days. Cells are then counted using a Beckman-Coulter Particle Counter. Through both stimulation of cytokine release and growth-stimulating effects on the stroma, CG53135 may be able to create conditions wherein surviving marrow stem cells can proliferate and expand. This can be tested in vitro by measuring the expansion of pluripotent CD34⁺ cells on stroma that has been treated with CG53135. Stromal cells are plated at subconfluent densities and either treated with CG53135 or left untreated for 24 hours. CD34⁺ cells are then plated on top of the fibroblast layer and allowed to grow for 2, 4 or 6 days. At each time point, floating cells are collected and counted to measure proliferation, as well as stained for CD34 and Lin expression to ascertain that the cells have not differentiated.

### 6.17. Example 17

### CG53135 Reduces the Incidence, Length and Severity of Radiation-Induced Diarrhea (N-438)

This study was performed to evaluate the activity of CG53135 against gastrointestinal injury induced by whole body irradiation as measured by diarrhea incidence and gut morphology (protein concentrations in this example were measured by UV absorbance).

Materials and Methods:

Dosing: Mice were weighed and then dosed with CG53135-05 E. coli purified product (4 or 16 mg/kg) or untreated. Dosing occurred as described in Tables 1 & 2. Each group of 20 animals was irradiated as per table below. All dosing of CG53135-05 E. coli purified product on day 0 was immediately after irradiation. No anesthesia was administered.

Intestinal Crypt Cell Damage Induction: Mice underwent whole body irradiation at a dose of 14 or 14.5 Gy delivered at a dose rate of 0.7 Gy/min. Animals were followed for diarrhea incidence throughout the study period. After 6 days, animals were sacrificed, and the intestinal tract of the mice was harvested for histological analysis.

Body Weight: Every day for the period of the study, each animal was weighed and its survival recorded, in order to assess possible differences in animal weight among treatment groups as an indication of response to exposure to ionizing radiation.

Animals Found Dead or Moribund: Animals were assessed 2×/day from Day 3 onwards in order to accurately assess diarrhea onset/progression and detect moribund animals prior to death. Such moribund animals were sacrificed by cervical dislocation. The ileum and mid-colon were removed and fixed in formalin, embedded in paraffin (1 animal per block, two tissues per block) for storage and future analysis/IHC if required. No tissue was removed from animals found dead.

TABLE 21
Study Design

| Group | Number of | | | Treatment | olume |
|---|---|---|---|---|---|
| Number | Animals | Induction | Treatment | Schedule* | (mL) |
| 1 | 20 males | 14 Gy | None | None | Adjust per body |
| | | Day 0 | | | weight |
| 2 | 20 males | 14 Gy | CG53135-05 E. coli | Day −1, 0, 1 | Adjust per body |
| | | Day 0 | purified product, | | weight |
| | | | 4 mg/kg, IP | | |
| | | | (qd × 3) | | |
| 3 | 20 males | 14 Gy | CG53135-05 E. coli | Day −1, 0, 1 | Adjust per body |
| | | Day 0 | purified product, | | weight |
| | | | 16 mg/kg, IP | | |
| | | | (qd × 3) | | |
| 4 | 20 males | 14 Gy | CG53135-05 E. coli | Day 1 | Adjust per body |
| | | Day 0 | purified product, | | weight |
| | | | 4 mg/kg, IP | | |
| | | | (q6h × 4) | | |
| 5 | 20 males | 14.5 Gy | None | None | Adjust per body |
| | | Day 0 | | | weight |
| 6 | 20 males | 14.5 Gy | CG53135-05 E. coli | Day −1, 0, 1 | Adjust per body |
| | | Day 0 | purified product, | | weight |
| | | | 4 mg/kg, IP | | |
| | | | (qd × 3) | | |
| 7 | 20 males | 14.5 Gy | CG53135-05 E. coli | Day −1, 0, 1 | Adjust per body |
| | | Day 0 | purified product, | | weight |
| | | | 16 mg/kg, IP | | |
| | | | (qd × 3) | | |

TABLE 22
Test Article Requirements

| | | | | | | | Conc. of | Desired | Volume | Volume of |
|---|---|---|---|---|---|---|---|---|---|---|
| | | Dose | | | | | stock | Conc of | of dosing | stock |
| | | (mg/kg) | | | Mass of | | solution | Dosing | solution | solution |
| Type of | | from | # of | # of | Animal | Admin vol | by A₂₈₀ | solution | required | required for |
| Solution | Group # | Conc A₂₈₀ | Animals | doses | (kg) | (mL/kg) | (mg/mL) | (mg/mL) | (mL) | dilution (mL) |
| CG53135 | 1 | 0 | 20 | 0 | 0.025 | 10 | 10.2 | 0.000 | 0.000 | 0.000 |
| CG53135 | 2 | 4 | 20 | 3 | 0.025 | 10 | 10.2 | 0.400 | 18.750 | 0.735 |
| CG53135 | 3 | 16 | 20 | 3 | 0.025 | 10 | 10.2 | 1.600 | 18.750 | 2.941 |
| CG53135 | 4 | 4 | 20 | 4 | 0.025 | 10 | 10.2 | 0.400 | 25.000 | 0.980 |
| CG53135 | 5 | 0 | 20 | 0 | 0.025 | 10 | 10.2 | 0.000 | 0.000 | 0.000 |
| CG53135 | 6 | 4 | 20 | 3 | 0.025 | 10 | 10.2 | 0.400 | 18.750 | 0.735 |
| CG53135 | 7 | 16 | 20 | 3 | 0.025 | 10 | 10.2 | 1.600 | 18.750 | 2.941 |

| | | |
|---|---|---|
| 140 | Total | 8.333 |

Results:

Excel spreadsheet attached with diarrhea scores and weights for animals irradiated with 14 or 14.5 Gy. Because the data from each radiation dose were very similar, only the analysis of the animals irradiated with 14 Gy is provided.

Weights: Mass specific growth rate was calculated by:

$$\frac{ln ( {M}_{f} ) - ln ( {M}_{i} )}{{T}_{{f}^{-}} {T}_{i}} = MSGR$$

Significance was calculated using One-way ANOVA and Dunnett's Multiple Comparison Test. No significant differences were seen between the changes in weight during the study between the groups (FIGS. 30(A) and (B)).

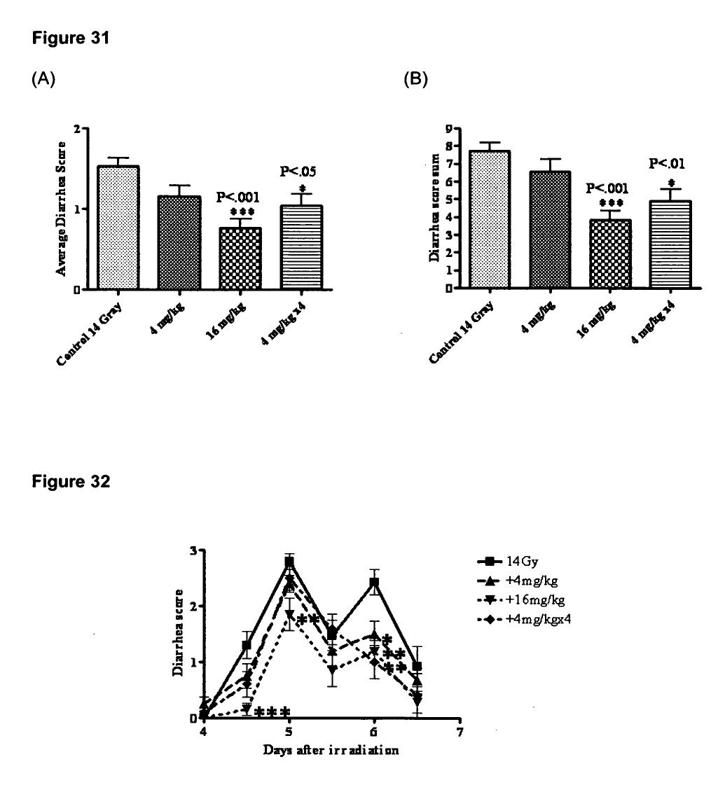

Diarrhea score: Mice were scored for severity of diarrhea on a scale of 0-3 twice a day for three days beginning at 4 days after irradiation. Average diarrhea score over three days as well as the sum of the diarrhea score over three days was measured and graphed. Significance was obtained by one-way ANOVA and Tukey's Multiple Comparison Test. (FIGS. 31(A) and (B)).

An analysis of for each day of observation was also made to determine differences at days of peak diarrhea. Significance was determined as described above (*-P<0.05, **-P<0.01, ***-P<0.001). (FIG. 32)

Conclusions:

Dosing animals with 16 mg/kg CG53135 at days −1, 0 and +1 respective to radiation resulted in a highly significant reduction in the incidence, length and severity of radiation-induced diarrhea. Dosing animals every 6 hours on day 1 with 4 mg/kg CG53135 also resulted in significant decrease in diarrhea incidence. The day of peak diarrhea was 5 days after radiation, at which point only the 16 mg/kg dose of CG53135 provided a significant decrease in diarrhea. There were no significant differences between the treatment groups in weight loss over the course of the study.

### 6.18. Example 18

### Manufacture of CG53135-05 and Pharmaceutical Formulations

Aiming for a construct that would be suitable for clinical development, untagged molecules were generated in a phage-free bacterial host. The codon-optimized, full-length, untagged molecule (CG53135-05) has the most favorable pharmacology profile and was used to prepare product for the safety studies and clinical trials.

6.18.1 Production Process and Pharmaceutical Formulations (Process 1)

CG53135-05 was expressed in Escherichia coli BLR (DE3) using a codon-optimized construct, purified to homogeneity, and characterized by standard protein chemistry techniques. The isolated CG53135-05 protein migrated as a single band (23 kilodalton) using standard SDS-PAGE techniques and stained with Coommassie blue. The CG53135-05 protein was electrophoretically transferred to a polyvinylidenefluoride membrane and the stained 23 kD band was excised from the membrane and analyzed by an automated Edman sequencer (Procise, Applied Biosystems, Foster City, Calif.); the N-terminal amino acid sequence of the first 10 amino acids was confirmed as identical to the predicted protein sequence.

Fermentation and Primary Recovery Recombinant

CG53135-05 was expressed using Escherichia coli BLR (DE3) cells (Novagen). These cells were transformed with full length, codon optimized CG53135-05 using pET24a vector (Novagen). A Manufacturing Master Cell Bank (MMCB) of these cells was produced and qualified. The fermentation and primary recovery processes were performed at the 100 L (i.e., working volume) scale reproducibly.

Seed preparation was started by thawing and pooling of 1-6 vials of the MMCB and inoculating 4-7 shake flasks each containing 750 mL of seed medium. At this point, 3-6 L of inoculum was transferred to a production fermentor containing 60-80 L of start-up medium. The production fermentor was operated at a temperature of 37° C. and pH of 7.1. Dissolved oxygen was controlled at 30% of saturation concentration or above by manipulating agitation speed, air sparging rate and enrichment of air with pure oxygen. Addition of feed medium was initiated at a cell density of 3040 AU (600 nm) and maintained until end of fermentation. The cells were induced at a cell density of 40-50 AU (600 nm) using 1 mM isopropyl-beta-D-thiogalactoside (IPTG) and CG53135-05 protein was produced for 4 hours post-induction. The fermentation was completed in 10-14 hours and about 100˜110 L of cell broth was concentrated using a continuous centrifuge. The resulting cell paste was stored frozen at −70° C.

The frozen cell paste was suspended in lysis buffer (containing 3M urea, final concentration) and disrupted by high-pressure homogenization. The cell lysate was clarified using continuous flow centrifugation. The resulting clarified lysate was directly loaded onto a SP-sepharose Fast Flow column equilibrated with SP equilibration buffer (3 M urea, 100 mM sodium phosphate, 20 mM sodium chloride, 5 mM EDTA, pH 7.4). CG53135-05 protein was eluted from the column using SP elution buffer (100 mM sodium citrate, 1 M arginine, 5 mM EDTA, pH 6.0). The collected material was then diluted with an equal volume of SP elution buffer. After thorough mixing, the SP Sepharose FF pool was filtered through a 0.2 μm PES filter and frozen at −80° C.

Purification of the Drug Substance

The SP-sepharose Fast Flow pool was precipitated with ammonium sulfate. After overnight incubation at 4° C., the precipitate was collected by bottle centrifugation and subsequently solubilized in Phenyl loading buffer (100 mM sodium citrate, 500 mM L-arginine, 750 mM NaCl, 5 mM EDTA, pH 6.0). The resulting solution was filtered through a 0.45 μM PES filter and loaded onto a Phenyl-sepharose HP column. After washing the column, the protein was eluted with a linear gradient with Phenyl elution buffer (100 mM sodium citrate, 500 mM L-arginine, 5 mM EDTA, pH 6.0). The Phenyl-sepharose HP pool was filtered through a 0.2 μm PES filter and frozen at −80° C. in 1.8 L aliquots.

Formulation and Fill/Finish

Four batches of purified drug substance were thawed for 24-48 hours at 2-8° C. and pooled into the collection tank of tangential flow ultrafiltration (TFF) equipment. The pooled drug substance was concentrated ˜5-fold via TFF, followed by about 5-fold diafiltration with the formulation buffer (40 mM sodium acetate, 0.2 M L-arginine, 3% glycerol). This buffer-exchanged drug substance was concentrated further to a target concentration of >10 mg/mL. Upon transfer to a collection tank, the concentration was adjusted to ˜10 mg/mL with formulation buffer. The formulated drug product was sterile-filtered into a sterile tank and aseptically filled (at 10.5 mL per 20 mL vial) and sealed. The filled and sealed vials were inspected for fill accuracy and visual defects. A specified number of vials were drawn and labeled for release assays, stability studies, safety studies, and retained samples. The remaining vials were labeled for the clinical study, and finished drug product was stored at −80±15° C.

The finished drug product is a sterile, clear, colorless solution in single-use sterile vials for injection. CG53135-05 E. coli purified product was formulated at a final concentration of 8.2 mg/mL (Table 23).

TABLE 23
Composition of Drug Product

| Component | Grade | Final concentration | | Amount per Liter |
|---|---|---|---|---|
| CG53135-05 E. coli | NA | 8.2 | mg/ml | 8.2 g |
| purified product | | | | |

Formulation Buffer

| | | | | |
|---|---|---|---|---|
| Sodium acetate | USP | 40 | mM | 5.44 g |
| (trihydrate) | | | | |
| L-arginine HCl | USP | 200 | mM | 42.132 g |
| Glycerol | USP | 3% | v/v | 30 mL |

| | | | |
|---|---|---|---|
| Acetic acid | USP | NA | QS to pH 5.3 |
| Water for injection | USP | NA | QS to 1 L |

The pharmacokinetics of optimally-formulated CG53135-05 E. coli purified product was assessed in rats following intravenous, subcutaneous, and intraperitoneal administration to compare exposure at active doses in animal models and predict exposure in humans. Intravenous administration of CG53135-05 E. coli purified product resulted in high plasma levels (maximum plasma level=19,680-47,252 ng/mL), which rapidly declined within the first 2 hours to 30-70 ng/mL; decreased exposure was observed following the third daily dose (maximum plasma level=5373-7453 ng/mL). Subcutaneous administration of CG53135-05 E. coli purified product resulted in slow absorption (maximum plasma level at 10 hours) and plasma levels of 40-80 ng/mL up to 48 hours after dosing; some accumulation in plasma was seen following the third daily dose. Intraperitoneal administration of CG53135-05 E. coli purified product resulted in slow absorption (maximum plasma level at 2-4 hours) and plasma levels of 40-70 ng/mL up to 10 hours after dosing; decreased exposure was seen following third daily dose. No significant gender differences were observed by any route of administration.

Safety of intravenous administration of CG53135-05 E. coli purified product (0.05, 5 or 50 mg/kg/day UV for 14 consecutive days) was assessed in a pivotal toxicology study in rats. There were no treatment-related findings in rats administered 0.05 mg/mL CG53135-05 E. coli purified product for 14 days. In rats administered 5 mg/kg CG53135 for 14 days, food consumption was reduced and body weight was decreased; while there were no treatment-related changes in organ weights, urinalysis, ophthalmology, or histopathology parameters in this dose group, there were treatment-related changes in hematology and clinical chemistry parameters in this treatment group. In rats administered 50 mg/kg CG53135-05 E. coli purified product for 12 days (estimated maximum plasma level of 20-30 fold higher than active dose), food consumption was reduced and body weight was markedly decreased; while there were no treatment-related changes in ophthalmology, there were significant treatment-related changes in organ weights, urinalysis, hematology, clinical chemistry, and histopathology in this treatment group.

Safety of intravenous administration of CG53135-05 E. coli purified product (0 or 10 mg/kg/day UV for 7 consecutive days) was further assessed in a safety pharmacology study in rhesus monkeys. There were no treatment-related clinical observations in animals administered 1 mg/kg CG53135-05 E. coli purified product for 7 days. In animals administered 10 mg/kg CG53135-05 E. coli purified product for 7 days, minor effects on body weight were noted and associated with qualitative observations of lower food consumption. There were no apparent treatment-related effects on hematology, clinical chemistry, ophthalmology, or electrophysiology in either dose group.

Stability of CG53135-05 Drug Substances

Stability studies on the CG53135-05 E coli purified product produced during cGMP manufacturing were performed. The analytical methods used as stability indicating assays for purified drug substance are listed in Table 24.

TABLE 24
Stability Assays for Drug Substance

| Assay | Stability Criteria |
|---|---|
| SDS-PAGE (Neuhoff stain) | >98% pure by densitometry |
| | (reduced and nonreduced) |
| RP-HPLC | Peak at 5.5 ± 1.0 min relative |
| | retention time |
| SEC-HPLC | >90% mono-disperse peak |
| Total protein by Bradford method | >0.2 mg/mL |
| Bioassay (BrdU) | PI₂₀₀ > 0.5 ng/mL and <20 ng/mL |
| pH | 5.8 ± 0.4 |
| Visual appearance | Clear and colorless |

PI₂₀₀ = concentration of CG53135-05 that results in incorporation of BrdU at 2 times the background

The SDS-PAGE, RP-HPLC, and Bradford assays are indicative of protein degradation or gross aggregation. The SEC-HPLC assay detects aggregation of the protein or changes in oligomerization, and the bioassay detects loss of biological activity of the protein. The stability studies for the purified drug substance were conducted at −80 to 15° C. with samples tested at intervals of 3, 6, 9, 12, and 24 months.

In one experiment, stability studies of finished drug product were conducted by Cambrex at −80 [symbol] 15° C. and −20 [symbol] 5° C. with samples tested at intervals of 1, 3, 6, 9, 12, and 24 months. Stability data collected after 1 month indicate that finished drug product is stable for at least 1 month when stored at −80 [symbol] 15° C. or at −20 [symbol] 5° C. (Table 25).

TABLE 25
Stability Data for Drug Product after 1-month interval

| Assay | Stability Criteria | Initial | −80 ± 15° C. | −20 ± 5° C. |
|---|---|---|---|---|
| RP-HPLC | Major peak | Major peak | Major peak | Major peak |
| | retention time ± 0.2 min | retention time ± 0.2 min | retention time ± 0.2 min | retention time ± 0.2 min |
| | relative to | relative | relative | relative |
| | Reference Standard | to Reference | to Reference | to Reference |
| | | Standard | Standard | Standard |
| SDS-PAGE | Major band | Pass | Pass | Pass |
| | migrates at about | | | |
| | 23 kDa; | | | |
| | nonreduced minor | | | |
| | band below major | | | |
| | band | | | |
| SEC-HPLC | >90% mono- | 100% | 100% | 100% |
| | disperse peak | | | |
| Bradford | 10 ± 0.2 mg/mL | 8.2 | 8.6 | 8.3 |
| Bioassay | PI₂₀₀ > 0.5 ng/mL | 4.14 ng/mL | 2.98 ng/mL | 1/45 ng/mL |
| | and <20 ng/mL | | | |
| Sterility | Pass (ie., no | Pass | NT | NT |
| | growth) | | | |
| pH | 5.3 ± 0.3 | 5.4 | 5.5 | 5.4 |
| Visual | Clear and colorless | Pass | Pass | Pass |
| appearance | solution | | | |

Lot # 02502001 was stored at −80 [symbol] 15° C. or at −20 [symbol] 5° C. at Cambrex and tested after 1 month; PI200=concentration of CG53135-05 that results in incorporation of BrdU at 2 times the background; Pass=results met stability criterion; NT=not tested.

In another experiment, samples of finished drug product were stored at −80 [symbol] 15° C. or stressed at 5 [symbol] 3° C., 25 [symbol] 2° C., or 37 [symbol] 2° C. and tested at various intervals for 1 month. Stability data indicate that finished drug product showed no significant instability after 1 month of storage at −80 [symbol] 15° C. or 5 [symbol] 3° C. When stressed at 25 [symbol] 2° C., finished drug product was stable for at least 48 hours; degradation was apparent after 1 week at this temperature. When stressed at 37 [symbol] 2° C., degradation of finished drug product was apparent within 4 hours.

6.18.2. Improved Pharmaceutical Formulations and Production Process of CG53135-05 (Process 2)

A new formulation was developed to meet the three requirements for a commercial product: (1) the minimal storage temperature should be 2-8° C. for ease of distribution; (2) product should be stable at the storage temperature for at least 18 months for a commercial distribution system; and (3) product should be manufactured by commercial scale equipment, and processes should be transferable to various commercial contract manufacturers.

The new formulation consists 10 mg/mL of the protein product produced by the process described in Section 6.2 (“Process 2 protein”) in 0.5 M arginine as sulfate salt, 0.05 M sodium phosphate monobasic, and 0.01% (w/v) polysorbate 80. The lyophilized product is projected to be stable for at least 18 months at 2-8° C. based on accelerated stability data. In contrast to the new formulation, the previous formulation as described in U.S. application Ser. No. 10/435,087 (see Section 6.18.1) is not possible to be lyophilized for the following reasons: firstly, the acidic component of the acetate buffer is acetic acid, which sublimes during lyophilization. This loss of acetic acid to lyophilization increases the pH to >7.5, which is far from the target pH of 5.3. Secondly, the glycerol has a collapse temperature of <−45° C., which renders this formulation not be able to be lyophilized commercially. Most of the commercial lyophilizers have a shelf temperature ranged from −45° C. to −50° C. with temperature variation of ±3° C.

Four unexpected properties of CG53135 were discovered and used to develop the new formulation: (1) high concentration of arginine, >0.4 M, increases the solubility to >30 mg/mL; (2) the use of sulfate salt of arginine increases the solubility by at least 2-6 folds; (3) the optimal concentration of sodium phosphate as a buffering salt is 50 mM, with a solubility of at least 1-2 fold increase in comparison with concentrations at 25, 75, and 100 mM; and (4) adding a surfactant during the diafiltration/ultrafiltration step minimizes the formation of aggregates. In development the lyophilized formulation, each component of the new formulation was evaluated for solubility individually. CG53135-05 was precipitated using the precipitate buffer (50 mM NaPi, 5 mM EDTA, 1 M L-Arginine HCl, 2.5 M (NH4)₂SO4). The precipitate was washed with 25 mM sodium phosphate buffer at pH 6.5 to remove the residual arginine and ammonium sulfate. The washed precipitate was then re-dissolved in the following respective buffers listed in the tables. The following are examples of data.

TABLE 26
High concentration of arginine, >0.4 M, increases the solubility
to >30 mg/mL

| | Solubility of | | | | |
|---|---|---|---|---|---|
| | Process 2 protein in mg/mL | | | | |
| Concenctration of | Batch | Batch | Batch | Batch | Batch |
| Arginine (M) | #1 | #2 | #3 | #4 | #5 |
| 0.05 | 0.7 | 0.6 | 0.5 | ND | ND |
| 0.10 | 1.4 | 0.6 | 1.2 | ND | ND |
| 0.15 | 2.2 | 1.6 | 2.2 | ND | ND |
| 0.20 | 3.0 | 4.7 | 4.3 | ND | ND |
| 0.30 | ND | ND | ND | 5.8 | ND |
| 0.35 | ND | ND | ND | 10.1 | ND |
| 0.40 | ND | ND | ND | 9.8 | ND |
| 0.45 | ND | ND | ND | 32.3 | ND |
| 0.50 | ND | ND | ND | 23.8* | 37 |

*The solubility was lower as there was not sufficient protein in the experiment to be dissolved.

TABLE 27
The use of the sulfate salt of arginine increases the solubility by at
least 2 to 6-fold.

| Concentration of | Solubility of Process 2 protein in mg/mL | | | | |
|---|---|---|---|---|---|
| sodium phosphate | Batch | Batch | Batch | Batch | Batch |
| monobasic* | #A | #B | #C | #D | #E |
| 100 mM | 3.78 | 2.8 | 2.4 | 2.9 | 2.47 |
| 75 mM | 4.06 | 2.5 | 2.6 | 3.0 | 2.38 |
| 50 mM | 5.47 | 4.7 | 3.3 | 4.3 | 4.81 |
| 25 mM | 4.01 | 2.4 | 2.6 | 2.4 | 3.59 |

All Formulation Contains 0.2 M Arginine.

An optimal concentration of the sodium phosphate as a buffering salt was observed (Table 28). The optimal concentration of sodium phosphate is 50 mM with a solubility of at least 1-2 fold increase in comparison with concentrations at 25, 75, or 100 mM.

TABLE 28
The optimal concentration of sodium phosphate
as a buffering salt is 50 mM

| | Solubility Increament of | |
|---|---|---|
| | Process 2 protein in | |
| | using Arginine Sulfate vs | |
| | Arginine Phosphate in mg/mL | |
| Formulation | Batch #K | Batch # J |
| 50 mM sodium phosphate monobasic and | 4.4 | 2.3 |
| 0.15M Arginine at pH 7 | | |
| 50 mM sodium phosphate monobasic and | 6.5 | 5.2 |
| 0.15M Arginine at pH 7 | | |

Table 29 shows a need to add a surfactant during the diafiltration/ultrafiltration step to minimize the formation of aggregates. The experiment was conducted by performing the ultrafiltration/diafiltration at 2.5 mg/mL CG53135-05 in 0.2 M Arginine and 0.05 M sodium phosphate buffer at pH 7.0. After exchanging with 7 volumes of the final buffer (0.5 M Arginine and 0.05 M sodium phosphate buffer at pH 7.0), the diafiltrate is concentrated to ˜20 mg/mL. The diafiltrate is then diluted with the final buffer to 12.5 mg/mL and lyophilized. Polysorbate 80 is added either before or after the diafiltration to a final concentration of 0.01%.

TABLE 29
Adding a surfactant during the diafiltration/ultrafiltration step minimizes
the formation of aggregates.

| Polysorbate | Process 2 protein | |
|---|---|---|
| added during | Concentration | Turbidity |
| ultrafiltration/diafiltration | (mg/mL) | (NTU) |
| Yes | 12.5 | 20.9 |
| No | 13.0 | 4.6 |

All formulation buffer contains 0.5 M arginine, 0.05 M sodium phosphate monobasic, and 0.01% polysorbate 80.

The new formulation has the following advantages: (1) a lyophilized product with a storage temperature of 2-8° C.; with a projected shelf-life of at least 18 months (2) the solubility of CG53135 is increased, allowing concentrations of >30 mg/mL to be obtained; and (3) the lyophilized product has a collapse temperature of −30° C. which can be easily lyophilized by the commercial equipment. The interactions between arginine, sulfate, phosphate, and surfactant and CG53135 were unexpected.

The improved process steps (Process 2) for the manufacturing of drug substance and drug product are described in Table 30, and each step is explained below.

TABLE 30
Manufacturing Process
Ampoule from WCB
↓
Seed Flask and Seed Fermenter 25 L - lnnoc
↓
Fermentation at 1500 L scale
↓
Homogenization + 0.033% PEI or a charged
heterogenous polymer
↓
Purification by SP Streamline
↓
Purification by PPG 650 M
↓
Cuno Filtration
↓
Purification by Phenyl Sepharose HP
↓
Concentration/Diafiltration addition of 0.01%
polysorbate 80 or Polysorbate 20
↓
Bottling - Drug Substance
↓
QC Testing and Release
↓
Sterile Vial Fill & Lyophilization
↓
Drug Product
↓
QC Testing and Release

Cell Bank: a Manufacturing Master Cell Bank (MMCB) in animal component free complex medium was used in an earlier Process. A second Manufacturing Master Cell Bank (MMCB) in animal component free chemically defined medium was derived from the first MMCB and a Manufacturing Working Cell Bank (MWCB) was made from the second MMCB. This MWCB was used in the manufacturing process as described in Table 30.

Inoculum Preparation: the initial cell expansion occurs in shake flasks. Seed preparation is done by thawing and pooling 2-3 vials of the MWCB in chemically defined medium and inoculating 3-4 shake flasks each containing 500 mL of chemically defined seed medium.

Seed and Final Fermentation: the shake flasks with cells in exponential growth phase (2.5-4.5 OD₆₀₀ units) are used to inoculate a single 25 L (i.e., working volume) seed fermenter containing the seed medium. The cells upon reaching exponential growth phase (3.0-5.0 OD600 units) in the 25 L seed fermenter are transferred to a 1500 L production fermenter with 780-820 L of chemically defined batch medium. During fermentation, the temperature is controlled at 37±2° C., pH at 7.1±0.1, agitation at 150-250 rpm and sparging with 0.5-1.5 (vvm) of air or oxygen-enriched air to control dissolved oxygen at 25% or above. Antifoam agent (Fermax adjuvant 27) is used as needed to control foaming in the fermenter. When the OD (at 600 nm) of culture reaches 25-35 units, additional chemically defined medium is fed at 0.7 g/kg broth/min initially and then with feed rate adjustment as needed. The induction for expression of CG53135-05 protein is started when OD at 600 nm reaches 135-165 units. After 4 hours post-induction the fermentation is completed. The final fermentation broth volume is approximately 1500 L. The culture is then chilled to 10-15° C.

Homogenization: the chilled culture is diluted with cell lysis buffer at the ratio of one part of fermentation broth to two parts of cell lysis buffer (50 mM sodium phosphate, 60 mM EDTA, 7.5 mM DTT, 4.5 M urea, pH 7.2. Polyethyleneimine (PEI), a flocculating agent is added to the diluted fermentation broth to a final PEI concentration at 0.033% (W/V). The cells are lysed at 10-15° C. with 3 passages through a high-pressure homogenizer at 750-850 bar.

Capture and Recovery: the chilled cell lysate is directly loaded in the upflow direction onto a pre-equilibrated Streamline SP expanded bed cation exchange column. During the loading, the bed expansion factor is maintained between 2.5-3.0 times the packed bed column volume. After loading, the column is flushed with additional Streamline SP equilibration buffer (100 mM sodium phosphate, 40 mM EDTA, 10 mM sodium sulfate, 3 M urea, pH 7.0) in the upflow direction. The column is then washed further with SP Streamline wash buffer (100 mM sodium phosphate, 5 mM EDTA, 25 mM sodium sulfate, 2.22 M dextrose, pH 7.0) in the downflow direction. The protein is eluted from the column with Streamline SP elution buffer (100 mM sodium phosphate, 5 mM EDTA, 200 mM sodium sulfate, 1 M L-arginine, pH 7.0) in the downflow direction.

PPG 650M Chromatography: the SP Streamline eluate is loaded on to a pre-equilibrated PPG 650 M, hydrophobic interaction chromatography column. The column is equilibrated and washed with 100 mM sodium phosphate, 200 mM sodium sulfate, 5 mM EDTA, 1 M Arginine pH 7.0. The column is further washed with 100 mM sodium phosphate, 5 mM EDTA, 0.9 M Arginine, pH 7.0. The product is eluted with 100 mM sodium phosphate, 5 mM EDTA, 0.2 M Arginine, pH 7.0.

CUNO Filtration: the PPG eluate is passed through an endotoxin binding CUNO 30ZA depth filter. The filter is flushed first with water for injection (WFI) and then with 100 mM sodium phosphate, 5 mM EDTA, 0.2 M Arginine, pH 7.0 (PPG eluate buffer). After flushing, the PPG eluate is passed through the filter. Air pressure is used to push the final liquid through the filter and its housing.

Phenyl Sepharose Chromatography: the CUNO filtrate is then loaded on to a pre-equilibrated Phenyl Sepharose hydrophobic interaction chromatography column. The column is equilibrated and washed with 100 mM sodium phosphate, 50 mM ammonium sulfate, 800 mM sodium chloride, 0.5 M Arginine, pH 7.0. The product is eluted with 50 mM sodium phosphate, 0.5 M Arginine, pH 7.0.

Concentration and Diafiltration: a 1% Polysorbate 80 is added to the Phenyl Sepharose eluate so that the final concentration in the drug substance is 0.01% (w/v). The eluate is then concentrated in an ultrafiltration system to about 2-3 g/L. The retentate is then diafiltered with 7 diafiltration volumes of 50 mM sodium phosphate, 0.5 M Arginine, pH 7.0 (Phenyl Sepharose elution buffer). After diafiltration the retentate is concentrated between 12-15 g/L. The retentate is filtered through a 0.22 μm filter and subsequently diluted to 10 g/L.

Bulk Bottling: the retentate from the concentration and diafiltration step is filtered through a 0.22 μm pore size filter into 2 L single use teflon bottles. The bottles are frozen at −70° C.

Drug Product/Vial: the Frozen Drug Substance is transported to Formatech Inc, MA from Diosynth—RTP, NC for the manufacture of the Drug Product. The bottles of frozen Drug Substance are thawed at ambient temperature. After the Drug Substance is completely thawed, it is pooled in a sterile container, filtered, filled into vials, partially stoppered, and lyophilized. After completion of the freeze-drying process, the vials are stoppered and capped. The lyophilized Drug Product is stored at 2-8° C.

The CG53135-05 reference standard was prepared at Diosynth RTP Inc, using a 140 L scale manufacturing process that was representative of the bulk drug substance manufacturing process (as described in the General Method of Manufacture). The reference standard was stored as 1 mL aliquots in 2 mL cryovials at −80° C.±15° C.

Purity of the final product was analyzed by SDS-PAGE, RP-HPLC, size exclusion-HPLC, and Western blot. Potency of the drug was measured by growth of NIH 3T3 cells in response to CG53135-05. All data indicated that the final product is suitable for clinical uses.

### 7. EQUIVALENCE

Those skilled in the art will recognize, or be able to ascertain using no more than routine experimentation, many equivalents to the specific embodiments of the invention described herein. Such equivalents are intended to be encompassed by the following claims.

Thus, while the preferred embodiments of the invention have been illustrated and described, it is to be understood that this invention is capable of variation and modification, and should not be limited to the precise terms set forth. The inventors desire to avail themselves of such changes and alterations which may be made for adapting the invention to various usages and conditions. Such alterations and changes may include, for example, different pharmaceutical compositions for the administration of the proteins according to the present invention to a mammal; different amounts of protein in the compositions to be administered; different times and means of administering the proteins according to the present invention; and different materials contained in the administration dose including, for example, combinations of different proteins, or combinations of the proteins according to the present invention together with other biologically active compounds for the same, similar or differing purposes than the desired utility of those proteins specifically disclosed herein. Such changes and alterations also are intended to include modifications in the amino acid sequence of the specific desired proteins described herein in which such changes alter the sequence in a manner as not to change the desired potential of the protein, but as to change solubility of the protein in the pharmaceutical composition to be administered or in the body, absorption of the protein by the body, protection of the protein for either shelf life or within the body until such time as the biological action of the protein is able to bring about the desired effect, and such similar modifications. Accordingly, such changes and alterations are properly intended to be within the full range of equivalents, and therefore within the purview of the following claims.

The invention and the manner and process of making and using it have been thus described in such full, clear, concise and exact terms so as to enable any person skilled in the art to which it pertains, or with which it is most nearly connected, to make and use the same.

1. A method of preventing or treating a disorder caused by an insult affecting rapidly proliferating tissue or one or more symptoms thereof comprising administering to a subject an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

2. The method of claim 1, wherein said insult is radiation exposure, exposure to a chemical agent or a microorganism, or a combination thereof.

3. A method of preventing or treating a disorder caused by radiation exposure or one or more symptoms thereof comprising administering to a subject an effective amount of a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

4. The method of claim 1 or 3, wherein the disorder is alimentary mucositis.

5. The method of claim 4, wherein the disorder is oral mucositis.

6. The method of claim 4, wherein the disorder is gastrointestinal mucositis.

7. The method of claim 1 or 3, wherein the disorder is a disorder of hematopoiesis.

8. The method of claim 7, wherein the disorder is anemia, leukopenia, thrombocytopenia, pancytopenia, or a clotting disorder.

9. The method of claim 1 or 3, wherein the disorder is bone marrow failure, graft-versus-host disease, radiation induced prostatitis, virginitis, urethritis, or a cardiovascular/central nervous system syndrome.

10. The method of claim 1 or 3, wherein said symptom is diarrhea, skin burn, sores, fatigue, dehydration, inflammation, hair loss, ulceration of alimentary tract mucosa, xerostomia, bleeding, or a combination thereof.

11. The method of claim 3, wherein an effective amount of said composition is administered to a subject who is going to be exposed to radiation, or a subject who has been exposed to radiation but prior to said disorder or a symptom thereof developed in said subject.

12. The method of claim 3, wherein an effective amount of said composition is administered to a subject who has been exposed to radiation and who has developed a disorder or a symptom thereof.

13. The method of claim 11 or 12, wherein the effective amount of said composition is administered to the subject in a single dose.

14. The method of claim 13, wherein the single dose of said composition is administered to a subject no more than 24 hours before the subject's exposure to radiation.

15. The method of claim 11 or 12, wherein the effective amount of said composition is administered to the subject in two or more doses.

16. The method of claim 15, wherein said composition is administered to the subject both before the subject's exposure to radiation and after the subject's exposure to radiation.

17. The method of claim 1 or 3, wherein said composition is administered by parenteral route.

18. The method of claim 17, wherein said administration is by intravenous, intramuscular, subcutaneous, intradermal, or intranasal administration.

19. The method of claim 1 or 3, wherein said composition comprises a protein comprising an amino acid sequence of SEQ ID NO:2.

20. The method of claim 1 or 3, wherein said composition comprises a protein comprising an amino acid sequence of SEQ ID NO:24.

21. The method of claim 1 or 3, wherein said composition comprises two or more proteins, wherein a first protein comprises an amino acid sequence of SEQ ID NO:2, and a second protein comprises an amino acid sequence of SEQ ID NO:24.

22. The method of claim 1 or 3, wherein said composition further comprising a pharmaceutically acceptable carrier.

23. The method of claim 22, wherein said composition comprises 0.02-0.2 M acetate, 0.5-5% glycerol, 0.2-0.5 M arginine-HCl, and 0.5-5 mg/ml of said isolated protein.

24. The method of claim 22, wherein said composition comprises 0.04 M acetate, 3% glycerol (volume/volume), 0.2 M arginine-HCl at pH 5.3, and 0.8 mg/ml of said isolated protein.

25. The method of claim 22, wherein said composition comprises 0.01-1 M arginine in a salt form, sulfobutyl ether Beta-cyclodextrin sodium, or sucrose, about 0.01-0.1 M sodium phosphate monobasic (NaH₂PO₄.H₂O), about 0.01%-0.1% weight/volume (“w/v”) polysorbate 80 or polysorbate 20, and about 0.005 mg/ml to about 50 mg/ml of said isolated protein.

26. The method of claim 25, wherein said arginine in a salt form is selected from the group consisting of arginine, arginine sulfate, arginine phosphate, and arginine hydrochloride.

27. The method of claim 25, wherein said arginine in a salt form, sulfobutyl ether Beta-cyclodextrin sodium or sucrose is of 0.01-0.7 M.

28. The method of claim 25, wherein said composition comprises an arginine in a salt form at a concentration of 0.5 M.

29. The method of claim 25, wherein said sodium phosphate monobasic is 0.05 M.

30. The method of claim 25, wherein said polysorbate 80 or polysorbate 20 is 0.01% (w/v).

31. The method of claim 25, wherein said isolated protein is at a concentration of 5-30 mg/ml.

32. The method of claim 25, wherein said isolated protein is at a concentration of 10 mg/ml.

33. The method of claim 1 or 3, wherein said subject is a mammal.

34. The method of claim 31, wherein said subject is a human.

35. A method of upregulating oxygen scavenging pathways comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

36. The method of claim 35, wherein said oxygen scavenging pathways comprise one or more superoxide dismutases (“SOD”).

37. The method of claim 36, wherein said superoxide dismutase is CuZnSOD or MnSOD.

38. The method of claim 35, wherein said oxygen scavenging pathways comprise genes selected from the group consisting of extracellular signal regulated kinase (“ERK”), adhesion related kinase (“AKT”), a superoxide dismutase, cyclooxygenase-2 (“COX2”), and NF-E2-related factor 2 (“NRF2”).

39. A method of stimulating secretion of an endogenous cytokine or an endogenous chemokine from a cell of a subject, wherein the method comprises administering to the subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

40. The method of claim 39, wherein said method stimulates secretion of an endogenous cytokine, and wherein said cytokine is interleukin-1 b (“IL-1 b”), IL-6, IL-7, IL-8, IL-11, or granulocyte-colony forming factor (“G-CSF”).

41. The method of claim 39, wherein said method stimulates secreting of an endogenous chemokine, and wherein said chemokine is chemokine (C-X-C motif) ligand 1 (“CXCL1”) or monocyte chemoattractant protein 1 (“MCP-1”).

42. A method of stimulating hematopoietic stem cell proliferation comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

43. A method of optimizing hematopoietic stem cell engraftment comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

44. A method of stimulating gastrointestinal stem cell proliferation comprising-administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

45. A method of recovery or protection of hematopoietic tissues from radiation damage comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

46. A method of recovery or protection of gastrointestinal tissues from radiation damage comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

47. A method of preventing or treating a disorder or one or more symptoms thereof in a subject, wherein said disorder or one or more symptoms thereof are associated with exposure to a vesicant agent, said method comprising administering to a subject a composition comprising an isolated protein selected from the group consisting of:
(a) a protein comprising an amino acid sequence of SEQ ID NOs:2, 4, 7, 10, 22, 24, 26, 28, 30, 32, 34, 36, 38, or 40;
(b) a protein with one or more amino acid substitutions to the protein of (a), wherein said substitutions are no more than 15% of the amino acid sequence of SEQ ID NOs:2, 4, 7, 10, 22, 24, 26, 28, 30, 32, 34, 36, 38, or 40, and wherein said protein with one or more amino acid substitutions retains cell proliferation stimulatory activity; and
(c) a fragment of the protein of (a) or (b), which fragment retains cell proliferation stimulatory activity.

48. The method of claim 47, wherein said agent is mustard gas.


SEQUENCE LISTING (61 sequences)

SEQ ID NO: 1 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 2 - protein, 211 aa, Homo sapiens
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 477 bp, Homo sapiens
  CDS (1)..(474)
atggctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 4 - protein, 158 aa, Homo sapiens
MAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 5 - DNA, 636 bp, Homo sapiens
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 6 - DNA, 540 bp, Homo sapiens
  CDS (1)..(537)
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgcgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 7 - protein, 179 aa, Homo sapiens
MAPLAEVGGFLGGLEGLGQPGAAQLAHLHGILRRRQLYCRTGFHLQILPDGSAQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 8 - DNA, 636 bp, Homo sapiens
atggctccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 9 - DNA, 540 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttag

SEQ ID NO: 10 - protein, 179 aa, Homo sapiens
MAPLAEVGGFLGGLEGLGQPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 11 - DNA, 54 bp, Homo sapiens
  CDS (1)..(54)
atggctcccttagccgaagtcgggggctttctgggcggcctggagggcttgggc

SEQ ID NO: 12 - protein, 18 aa, Homo sapiens
MAPLAEVGGFLGGLEGLG

SEQ ID NO: 13 - DNA, 63 bp, Homo sapiens
  CDS (1)..(63)
gagcggccgccgctgctgggcgagcgcaggagcgcggcggagcggagcgcgcgcggcgggccg

SEQ ID NO: 14 - protein, 21 aa, Homo sapiens
ERPPLLGERRSAAERSARGGP

SEQ ID NO: 15 - DNA, 63 bp, Homo sapiens
  CDS (1)..(63)
cgcaggtattttgtggcacttaacaaagacggaactccaagagatggcgccaggtccaagagg

SEQ ID NO: 16 - protein, 21 aa, Homo sapiens
RRYFVALNKDGTPRDGARSKR

SEQ ID NO: 17 - DNA, 60 bp, Homo sapiens
  CDS (1)..(60)
cctagaccagtggatccagaaagagttccagaattgtacaaggacctactgatgtacact

SEQ ID NO: 18 - protein, 20 aa, Homo sapiens
PRPVDPERVPELYKDLLMYT

SEQ ID NO: 19 - DNA, 51 bp, Homo sapiens
  CDS (1)..(51)
atgaacgacaagggcgagctgtacggcagcgagaagctgaccagcgagtgc

SEQ ID NO: 20 - protein, 17 aa, Homo sapiens
MNDKGELYGSEKLTSEC

SEQ ID NO: 21 - DNA, 633 bp, Homo sapiens
  CDS (1)..(633)
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaagaacctactgatgtacact

SEQ ID NO: 22 - protein, 211 aa, Homo sapiens [sequence omitted]

SEQ ID NO: 23 - DNA, 630 bp, Homo sapiens
  CDS (1)..(627)
ccgctggctgaagttggtggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 24 - protein, 209 aa, Homo sapiens
PLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 25 - DNA, 612 bp, Homo sapiens
  CDS (1)..(609)
ggtttcctgggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 26 - protein, 203 aa, Homo sapiens
GFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 27 - DNA, 603 bp, Homo sapiens
  CDS (1)..(600)
ggcggtctggagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 28 - protein, 200 aa, Homo sapiens
GGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 29 - DNA, 594 bp, Homo sapiens
  CDS (1)..(591)
gagggtctgggtcagcaggttggttctcacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 30 - protein, 197 aa, Homo sapiens
EGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 31 - DNA, 567 bp, Homo sapiens
  CDS (1)..(564)
cacttcctgctgccgccggctggtgaacgtccgccactgctgggtgaacgtcgctccgcagctgaacgctccgctcgtggtggcccgggtgctgctcagctggctcacctgcatggtatcctgcgtcgccgtcagctgtactgccgtactggtttccacctgcagatcctgccggatggttctgttcagggtacccgtcaggaccactctctgttcggtatcctggaattcatctctgttgctgttggtctggtttctatccgtggtgttgactctggcctgtacctgggtatgaacgacaaaggcgaactgtacggttctgaaaaactgacctctgaatgcatcttccgtgaacagtttgaagagaactggtacaacacctactcttccaacatctacaaacatggtgacaccggccgtcgctacttcgttgctctgaacaaagacggtaccccgcgtgatggtgctcgttctaaacgtcaccagaaattcacccacttcctgccgcgcccagttgacccggagcgtgttccagaactgtataaagacctgctgatgtacacctaa

SEQ ID NO: 32 - protein, 188 aa, Homo sapiens
HFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 33 - DNA, 402 bp, Homo sapiens
  CDS (1)..(402)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtcgac

SEQ ID NO: 34 - protein, 134 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVD

SEQ ID NO: 35 - DNA, 447 bp, Homo sapiens
  CDS (1)..(447)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 36 - protein, 149 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 37 - DNA, 396 bp, Homo sapiens
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatgaagacactggccgcaggtattttgtggcacttaacaaagacggaactccaagagatggcgccaggtccaagaggcatcagaaatttacacatttcttacctagacca

SEQ ID NO: 38 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHEDTGRRYFVALNKDGTPRDGARSKRHQKFTHFLPRP

SEQ ID NO: 39 - DNA, 396 bp, Homo sapiens
  CDS (1)..(396)
atcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagacca

SEQ ID NO: 40 - protein, 132 aa, Homo sapiens
ILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRP

SEQ ID NO: 41 - DNA, 537 bp, Homo sapiens
atggctcccttagccgaagtcgggggctttctgggcggcctggagggcttgggccagccgggggcagcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacact

SEQ ID NO: 42 - DNA, 27 bp, Artificial Sequence
  Description of Artifical Sequence Primer/Probe
ttcaaatgagattgtgggaaaattgct

SEQ ID NO: 43 - DNA, 24 bp, Artificial Sequence
  Description of Artifical Sequence Primer/Probe
agatcatctctgcctgagtatctt

SEQ ID NO: 44 - DNA, 17 bp, Artificial Sequence
  Description of Artifical Sequence Primer/Probe
gtgccggccaaggacag

SEQ ID NO: 45 - DNA, 21 bp, Artificial Sequence
  Description of Artifical Sequence Primer/Probe
cgttaagacatcaggctccag

SEQ ID NO: 46 - DNA, 20 bp, Artificial Sequence
  Description of Artifical Sequence Primer/Probe
tggccgatgtgtctattgaa

SEQ ID NO: 47 - DNA, 20 bp, Artificial Sequence
  Description of Artifical Sequence Primer/Probe
gggcctcagactacatccaa

SEQ ID NO: 48 - DNA, 20 bp, Artificial Sequence
  Description of Artifical Sequence Primer/Probe
ctggacaaacctcagcccta

SEQ ID NO: 49 - DNA, 20 bp, Artificial Sequence
  Description of Artifical Sequence Primer/Probe
ctgatttggacaagcagcaa

SEQ ID NO: 50 - DNA, 28 bp, Artificial Sequence
  Description of Artifical Sequence Primer/Probe
tccatttgtaccgaaacaccccgctcac

SEQ ID NO: 51 - DNA, 30 bp, Artificial Sequence
  Description of Artifical Sequence Primer/Probe
caaacattcccccaaaggagcagctctcag

SEQ ID NO: 52 - DNA, 21 bp, Artificial Sequence
  Description of Artifical Sequence Primer/Probe
atggatttgattgacatactt

SEQ ID NO: 53 - DNA, 21 bp, Artificial Sequence
  Description of Artifical Sequence Primer/Probe
ctagtttttcttaacatctgg

SEQ ID NO: 54 - DNA, 24 bp, Artificial Sequence
  Description of Artifical Sequence Primer/Probe
aaggtactacttatcgagaatgtg

SEQ ID NO: 55 - DNA, 24 bp, Artificial Sequence
  Description of Artifical Sequence Primer/Probe
gtcaggctcgatgtcaatggtctg

SEQ ID NO: 56 - DNA, 20 bp, Artificial Sequence
  Description of Artifical Sequence Primer/Probe
ggacttcgagcaagagatgg

SEQ ID NO: 57 - DNA, 20 bp, Artificial Sequence
  Description of Artifical Sequence Primer/Probe
agcactgtgttggcgtacag

SEQ ID NO: 58 - DNA, 27 bp, Artificial Sequence
  Description of Artifical Sequence Primer/Probe
gggaattcagcgtgactttggtctttt

SEQ ID NO: 59 - DNA, 28 bp, Artificial Sequence
  Description of Artifical Sequence Primer/Probe
gcggatccgagcaggcggcaatctgtaa

SEQ ID NO: 60 - DNA, 19 bp, Artificial Sequence
  Description of Artifical Sequence Primer/Probe
gcatccatgaaactacatt

SEQ ID NO: 61 - DNA, 19 bp, Artificial Sequence
  Description of Artifical Sequence Primer/Probe
cacttgcggtgcacgatgg